(19) United States
(12) Patent Application Publication
(10) Pub. No.: US 2006/0078542 A1
(43) Pub. Date: Apr. 13, 2006

(54) Gel-based delivery of recombinant adeno-associated virus vectors

(76) Inventors: Cathryn S. Mah, Gainesville, FL (US); Thomas J. Fraites JR., Raleigh, NC (US); Barry J. Byrne, Gainesville, FL (US)

Correspondence Address: HAYNES AND BOONE, LLP, 901 MAIN STREET, SUITE 3100, DALLAS, TX, 75202, US

(21) Appl. No.: 11/055,497

(22) Filed: Feb. 10, 2005

Related U.S. Application Data
(60) Provisional application No. 60/543,508, filed on Feb. 10, 2004.

(51) Int. Cl. A61K 48/00; A61K 9/14

(52) U.S. Cl. 424/93.2; 424/488

(57) ABSTRACT

Disclosed are water-soluble gel-based compositions for the delivery of recombinant adeno-associated virus (rAAV) vectors that express nucleic acid segments encoding therapeutic constructs including peptides, polypeptides, ribozymes, and catalytic RNA molecules, to selected cells and tissues of vertebrate animals. Also disclosed are gel-based rAAV compositions are useful in the treatment of mammalian, and in particular, human diseases, including for example, cardiac disease or dysfunction, and musculoskeletal disorders and congenital myopathies, including, for example, muscular dystrophy, acid maltase deficiency (Pompe's disease), and the like. In illustrative embodiments, the invention provides rAAV vectors comprised within a biocompatible gel composition for enhanced viral delivery/transfection to mammalian tissues, and in particular to vertebrate muscle tissues such as a human heart or diaphragm tissue.

### 1.0 BACKGROUND OF THE INVENTION

The present application claims priority to U.S. Provisional Application Ser. No. 60/543,508, filed Feb. 10, 2004, the entire contents of which is specifically incorporated herein by reference. The United States government has certain rights in the present invention pursuant to grant NIDDK P01 DK58327-03 from the National Institutes of Health.

1.1 Field of the Invention

The present invention relates generally to the fields of molecular biology and virology, and in particular, to water-soluble gel-based compositions for the delivery of recombinant adeno-associated virus (rAAV) vectors express nucleic acid segments encoding therapeutic constructs including peptides, polypeptides, ribozymes, and catalytic RNA molecules, to selected cells and tissues of vertebrate animals. In particular, these gel-based rAAV compositions are useful in the treatment of mammalian, and in particular, human diseases, disorders, and dysfunctions. In illustrative embodiments, the invention concerns the use of rAAV vectors comprised within a gel suspension for delivery to mammalian tissues, and in particular muscle tissues of the vertebrate diaphragm. These gel-based rAAV compositions may be utilized in a variety of investigative, diagnostic and therapeutic regimens, including the prevention and treatment of musculoskeletal disorders and congenital myopathies including, for example muscular dystrophy and the like. Methods and compositions are provided for preparing gel-based rAAV vector compositions for use in the preparation of medicaments useful in central and targeted gene therapy of diseases, disorders, and dysfunctions in an animal, and in humans in particular.

1.2 Description of the Related Art

Previous studies of gene transfer to the diaphragm in rodents have been attempted via delivery of non-viral or adenoviral gene transfer vectors. Liu et al. (2001) recently described a method for systemic delivery of plasmid DNA carrying the full-length dystrophin gene with subsequent targeting to the diaphragm in max mice, a mouse strain with X-linked muscular dystrophy that mimics the diaphragmatic degeneration observed in Duchenne muscular dystrophy (Stedman et al, 1991). In that study, which used no carrier molecules, plasmid DNA was delivered intravenously via tail vein followed by transient (8-second) occlusion of the vena cava at the level of the diaphragm. High levels of gene expression were measured in diaphragm homogenates the next day and for 180 days (Liu et al., 2001), implicating dwell time as potentially the most significant determinant of successful gene transfer to the diaphragm with naked DNA. Two reports (Petrof et al., 1995; Yang et al., 1998) also indicated successful direct injection of recombinant adenoviruses carrying a mini-dystrophin gene to the diaphragms of mdx mice. Both studies demonstrated high levels of expression focally, presumably due to the delivery method. Transient gene expression, due to vector-related, dose-dependent inflammation, made assessment of the uniformity of gene expression difficult, but even with focal expression the authors observed measurable improvements in contractile function. More recently, Sakamoto et al. (2002) have developed an mdx strain that is transgenic for a micro-dystrophin construct, which is within the packaging capacity of rAAV.

1.3 Deficiencies in the Prior Art

Currently, there are limited pharmacological approaches to providing sufficiently high titers of rAAV particles to certain cells and tissues in affected mammals. A major hurdle in most current human gene therapy strategies is the ability to transduce target tissues at very high efficiencies that ultimately lead to therapeutic levels of transgene expression. This is particular true for tissues such as the vertebrate diaphragm.

Many such methods introduce undesirable side-effects, and do not overcome the problems associated with traditional modalities and treatment regimens for such conditions. Thus, the need exists for an effective treatment that circumvents the adverse effects and provides more desirable results, with longer acting effects, and improved compliance in both human and veterinary patients.

### 2.0 SUMMARY OF THE INVENTION

The present invention overcomes these and other limitations inherent in the prior art by providing a new gel-based method for delivery of recombinant adeno-associated virus (AAV) vectors. In illustrative embodiments of this new system, recombinant AAV vectors are mixed with a water-soluble glycerin-based gel and applied directly to the target tissue. The gel provides increased exposure time of target cells to the vector, thereby increasing the efficiency of transduction in the targeted areas.

In one embodiment, the invention discloses and claims a composition comprising a recombinant adeno-associated viral vector and a water-soluble biocompatible gel. The rAAV vector may comprise rAAV virions, or rAAV particles, or pluralities thereof. Preferably the gel comprises a matrix, a hydrogel, or a polymer, which may optionally be cross-linked, stabilized, chemically conjugated, or otherwise modified. The gel may optionally be a sustained release formulation, or may be biodegradable. Such gels may comprise one or more polymers, viscous contrast agents (such as iodixanol) or other viscosity- or density-enhancing agents, including for example, polysaccharides, including sucrose-based media (e.g., sucrose acetate isobutyrate).

The composition may comprise a biocompatible gel such as one or more of the commercially-available gel compounds including for example, SAF-Gel, Duoderm Hydroactive Gel, Nu-Gel; Carrasyn (V) Acemannan Hydrogel, Elta Hydrogel or K-Y Sterile Gel. In preferred embodiments, the gel comprises glycerin, gelatin, or alginate, or derivatives, mixtures, or combinations thereof. In typical formulations developed for administration to a mammal, and particularly for compositions formulated for human administration, the gel may comprise substantially all of the non-viral weight of the composition, and may comprise as much as about 98% or 99% of the composition by weight. This is particular desirous when substantially non-fluid, or substantially viscous formulations of the rAAV particles, vectors, or virions are preferred. When slightly less viscous, or slightly more fluid compositions are desired, the biocompatible gel portion of the composition may comprise at least about 50% by weight, at least about 60% by weight, at least about 70% by weight, or even at least about 80% or 90% by weight of the composition. Of course, all intermediate integers within these ranges. are contemplated to fall within the scope of this disclosure, and in certain embodiments, even more fluid (and consequently less viscous) gel/viral compositions may be formulated, such as for example, those in which the gel or matrix component of the mixture comprises not more than about 50% by weight, not more than about 40% by weight, not more than about 30% by weight, or even those than comprise not more than about 15% or 20% by weight of the composition In such exemplary formulations, the recombinant adeno-associated viral vectors may comprise either wild-type or genetically-modified rAAV vectors, including for example, recombinant vectors obtained from an AAV serotype 1 strain (rAAV1), an AAV serotype 2 strain (rAAV2), an AAV serotype 3 strain (rAAV3), an AAV serotype 4 strain (rAAV4), an AAV serotype 5 strain (rAAV5), an AAV serotype 6 strain (rAAV6), an AAV serotype 7 strain (rAAV7), an AAV serotype 8 strain (rAAV8), or an AAV serotype 9 strain (rAAV9), or combinations of two or more of such vectors. Optionally the composition may comprise a second viral or non-viral vector, or other therapeutic component as deemed necessary for the particular application. Such viral vectors may include, but are not limited to, Adenoviral vectors (AV), Herpes simplex virus vectors (HSV), and others such like that are known in the art.

Preferably, in almost all cases, the recombinant adeno-associated viral vectors formulated in the biocompatible water-soluble gels and matrices disclosed here will comprise at least a first nucleic acid segment that encodes one or more therapeutic agents, and that is expressed in a mammalian cell suitably comprising the rAAV vector. Such therapeutic agents may comprise one or more nucleic acids, peptides, polypeptides, proteins, antibodies, antigens, epitopes, binding domains, antisense molecules, or catalytic RNA molecules (such as, for example, a hammerhead or hairpin ribozyme having specificity for a target polynucleotide within the selected host cells into which the rAAV compositions are delivered and/or expressed.

In certain embodiments, the gel compositions my further optionally comprise one or more pharmaceutical excipients, diluents, buffers, or such like, and may further comprise one or more lipid complexes, liposomes, nanocapsules, microspheres, or other agents which may enhance, stabilize, or facilitate uptake of the rAAV vectors by suitable cells or tissue types either in vitro or ex vivo, or within the body of the animal into which the rAAV vector compositions are introduced (in situ and in vivo). In important embodiments, the compositions of the present invention are formulated and intended for use in therapy, particularly in the therapy of mammals, including humans, domesticated livestock, and animals under the care of a veterinarian or other trained animal medicine practitioner, that have, are suspected of having, or are at risk for developing one or more diseases, disorders, or dysfunctions, including for example, musculoskeletal diseases and congenital myopathies, such as muscular dystrophy and related conditions.

The invention also provides kits for diagnosing, preventing, treating or ameliorating the symptoms of a diseases or disorder in a mammal. Such kits generally will comprise one or more of the water-soluble gell-based rAAV compositions as disclosed herein, and instructions for using said kit. The invention also contemplates the use of one or more of the disclosed compositions, in the manufacture of medicaments for treating, abating, reducing, or ameliorating the symptoms of a disease, dysfunction, or disorder in a mammal, such as a human that has, is suspected of having, or at risk for developing a musculoskeletal disorder or a congenital myopathy such as muscular dystrophy.

The invention also contemplates the use of one or more of the disclosed compositions, in the manufacture of compositions and/or medicaments for increasing the bioavailability, cellular binding, cellular uptake, or increasing or altering the tissue-specificity for a particular AAV-derived vector used in a particular animal or cell type. The compositions of the invention are contemplated to be particularly useful in improving the transformation efficiency, and/or increasing the titer of a particular rAAV vector for a given cell, tissue, or organ into which introduction of rAAV vectors is desired. The inventors have demonstrated that the use of the disclosed gel-based delivery vehicles can substantially improve the efficiency of transformation for various cell and/or tissue types. As such, the compositions disclosed herein are particularly useful in providing a means for improving cellular uptake or viral infectivity of a given cell or tissue type.

Methods are also provided by the present invention for administering to a mammal in need thereof, an effective amount of at least a first therapeutic agent in an amount and for a time sufficient to provide the mammal with one or more of the disclosed compositions via introduction of such compositions into suitable cells or tissues of the mammal, either in vitro, in vivo, in situ, or ex situ. Such methods are particularly desirable in the treatment, amelioration, or prevention of diseases, including myopathies such as muscular dystrophy and the like, for which the inventors contemplate that administration of sufficiently high titers of suitable therapeutic rAAV gel-based compositions directly into the diaphragm of affected individuals will afford expression of one or more suitable therapeutic agents necessary to facilitate treatment.

In these and all other therapeutic embodiments, the rAAV compositions may be introduced into cells or tissues by any means suitable, including for example, by systemic or localized injection, or by other means of viral delivery as may be known in the art, including, but not limited to topical, intravenous, intramuscular, intraorgan, or transabdominal delivery, or other means such as transdermal administration.

In one embodiment, the present invention provides for a composition that comprises, consists essentially of, or consists of: a recombinant adeno-associated viral vector that comprises a nucleic acid segment that encodes a mammalian therapeutic agent; and a water-soluble biocompatible gel, gel matrix, sol, or sol matrix. Such biocompatible gels, sols and matrices may comprise, consist essentially of, or consist of a biogel, a hydrogel, a polymer, a monosaccharide, a polysaccharide, an oligosaccharide, or a viscosity agent. Exemplary viscosity agents include viscous contrast agents such as iodixanol, or a saccharide-containing component such as a fructose, sucrose, lactose, glucose, or arabinose-containing compound. In illustrative embodiments, the biocompatible gel may comprise, consist essentially of, or consist of glycerin or a glycerin-derived compound, a gelatin or a gelatin-derived compound, or an alginate or an alginate-derived compound. Exemplary biocompatible gels which are commercially available include, but are not limited to, SAF-Gel, Duoderm Hydroactive Gel, Nu-Gel; Carrasyn (V) Acemannan Hydrogel, Elta Hydrogel and K-Y Sterile Gel, to name only a few. The inventors contemplate virtually any gel or matrix material will be useful in the practice of the invention so long as it is not deleterious to the mammalian host cells into which it is introduced, or to the particular viral particles or virions which are suspended in the gel. In some instances, it may be desirable to use a plurality of two or more different gel materials to formulation the composition. One or more of such biocompatible gels may be partially, or substantially entirely cross-linked by one or more cross-linking agents. Alternatively, one or more of such biocompatible gels may be partially, or substantially entirely conjugated to one or more additional molecules, such as dyes, ligands, carriers, liposomes, lipoproteins, or other chemical or pharmaceutical compounds.

Preferably in the practice of the invention, the number of viral vectors, viral particles, and/or virions comprised within the biocompatible gel will be at least on the order of about 1 or 2×10¹¹ AAV particles per milliliter, and more preferably on the order of about 3 or 4×10¹¹ AAV particles per milliliter, and more preferably still, on the order of about 7 or 8×10¹¹ AAV particles per milliliter. Alternatively, when a higher titer of particles is desired, the compositions of the present invention may comprise about 1×10¹² AAV particles per milliliter, 2×10¹² AAV particles per milliliter, 5×10¹² AAV particles per milliliter, 7×10¹² AAV particles per milliliter, or even about 1×10¹³ AAV particles per milliliter, 3×10¹³ AAV particles per milliliter, or 5×10¹³ or so AAV particles per milliliter.

In the practice of the invention, the biocompatible gel may comprise at least about 50% by weight of the composition, at least about 55%, or at least about 60% by weight of the composition. In other embodiments, when an even more viscous medium is preferred, the biocompatible gel may comprise at least about 65%, at least about 70%, at least about 75%, or even at least about 80% or so by weight of the composition. In highly concentrated samples, the biocompatible gel may comprise at least about 85%, at least about 90% or at least about 95% or more by weight of the viral composition. The compositions may optionally also comprise one or more biological diluents or buffers, or some other pharmaceutically-acceptable vehicle or excipient.

The mammalian therapeutic agents used in the practice of the invention may be a nucleic acid segment that encodes a mammalian peptide, polypeptide, enzyme, or protein, or alternatively, may comprise a polynucleotide sequence that encodes either an antisense or a catalytic RNA molecule (ribozyme).

Preferably, the mammalian therapeutic agent is a peptide, polypeptide, enzyme, protein, antisense, or ribozyme that can be expressed in one or more human tissues, and particularly in human muscle tissues, such as diaphragm and cardiac muscle tissues.

Examples of mammalian therapeutic agents contemplated for use in the present invention are those agents that treat, prevent, or ameliorate the symptoms of one or more muscular, neuromuscular, myopathic, or neuropathic diseases, disorders, dysfunctions or abnormalities. Examples of such polypeptides include, but are not limited to, biologically-active mammalian (and particularly human) acid α-glucosidase (GAA), dystrophin, or α-1 antitrypsin polypeptide.

The invention also provides therapeutic and diagnostic kits that typically comprise one or more of the AAV gel-based compositions and instructions for using the kit in particular regimens or modalities. Likewise, the invention provides uses of the compositions in a method for providing a biologically-effective amount of a therapeutic agent to a tissue site of a mammal in need thereof. The method generally involves at least the step of providing one or more of the disclosed AAV gel-based therapeutic compositions to a mammal in need thereof in an amount and for a time effective to provide a biologically-effective amount of the encoded therapeutic agent to particular cells, tissues, or organ(s) of the animal being treated. Typical modes of administration of the compositions include, for example, transfection, systemic administration, or by direct, indirect, or localized injection to a cell, tissue, or organ of the mammal using methodologies that are routine to those practicing in the related art. In preferred embodiments, the mammal is a human that has, is suspected of having, or at risk for developing a musculoskeletal disorder, a glycogen storage disease, a neuromuscular disorder, a neuropathic condition, or a congenital myopathy, injury, or trauma. Exemplary conditions for which treatment using one of more of the disclosed AAV compositions is highly preferred include, for example, muscular dystrophy (including, for example, the Duchenne Becker form), cardiac injury, infart, trauma, ischemia, or hypertrophy, or metabolic disorders such as acid maltase deficiency (also known as Pompe's Disease).

The invention also provides for uses of the compositions in a method for treating or preventing a musculoskeletal disease or dysfunction, or a congenital myopathy in a mammal. The method generally involves at least the step of providing to such a mammal, one or more of the AAV gel-based compositions, in an amount and for a time effective to treat or prevent the musculoskeletal disease or dysfunction, or congenital myopathy in the animal. In preferred embodiments, the mammal is a human that has, is suspected of having, or is at risk for developing musculoskeletal disease or congenital myopathy.

In another embodiment, the invention provides for uses of the disclosed AAV gel-based compositions in a method of expressing in cells of a mammalian heart or diaphragm muscle, a nucleic acid segment that encodes an exogenously-provided mammalian therapeutic agent. In an overall and general sense, the method generally comprises at least the step of injecting into heart or diaphragm tissue, one or more of the disclosed AAV-therapeutic gene constructs in an amount and for a time effective to express the exogenously-provided mammalian therapeutic agent.

The invention also provides in another embodiment, a use for the disclosed AAV gel-based compositions in a method for treating or ameliorating the symptoms of a congenital myopathy in a mammal. This method generally comprises administering to a mammal in need thereof, one or more of the disclosed AAV-therapeutic gene constructs, in an amount and for a time sufficient to treat or ameliorate the symptoms of the congenital myopathy in the mammal.

Also disclosed are methods and compositions for expressing a biologically-effective amount of an exogenously-supplied therapeutic polynucleotide construct that encodes a therapeutic agent such as a peptide, polypeptide or protein in a mammalian diaphragm, heart, or muscle cell. The method generally involves: introducing into a population of mammalian diaphragm, heart, or muscle cells, an amount of an AAV gel-based composition, for a time effective to express a biologically-effective amount of the exogenously-supplied therapeutic agent in the cells that were transfected with he composition and that express the heterologous gene to produce the encoded polypeptide product in the diaphragm, heart or muscle cells.

In such methods, the therapeutic peptide, polypeptide or protein may be an antibody, an antigenic fragment, an enzyme, a kinase, a protease, a glucosidase (including for example human acid α- and β-glucosidases), a glycosidase (including for example human acid α- and β-glycosidases), a nuclease, a growth factor, a tissue factor, a myogenic factor, a neurotrophic factor, a neurotrophin, a dystrophin, an interleukin, or a cytokine.

### 3.0 BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1A, FIG. 1B and FIG. 1C show an illustrative gel-based delivery preparation. FIG. 1A shows rAAV vectors mixed in a 2-mL microcentrifuge tube and then centrifuged briefly. FIG. 2B shows the tube is punctured using a 22-gauge needle, creating an aperture through which the virus-gel suspension can be propelled. FIG. 1C shows a plunger from a standard 3 cc syringe is used to push the vector from the tube, enabling its application to the diaphragmatic surface. The oblique, bottom surface of the microcentrifuge tube is used to distribute the vector-gel suspension evenly on the surface.

FIG. 2A and FIG. 2B show free virus and gel-based delivery of rAAV-βgal vectors based on AAV serotypes 1, 2, and 5. Adult wild-type mice (129X1×C57BL/6) were treated with 1×10¹¹ particles of rAAV-βgal, with virus either directly applied to the diaphragm or applied using the gel-based method. The animals were sacrificed six weeks later and tissues were collected and assayed for β-galactosidase activity. FIG. 2A shows representative histochemical (X-gal) stained diaphragm segments from treated animals. Each row corresponds to the respective serotype into which the recombinant vector genome was packaged (AAV1, 2, and 5, respectively). The columns represent application of free virus (left) or virus-gel suspension (right) to the abdominal surface of the diaphragms, respectively. Note the intense blue staining in both columns for vector virions packaged using the rAAV1 capsid (top row), with increased intensity using the gel-based method (top row, right panel). FIG. 2B shows quantitative assay of β-galactosidase activity from the same animals. The bars represent mean±SEM acid α-glucosidase (GAA) activity for three mice in each group.

FIG. 3A and FIG. 3B show an illustrative embodiment of the invention in which rAAV1-hGAA-mediated transduction of the diaphragms of Gaa^{-/-} mice was demonstrated. FIG. 3A shows adult Gaa^{-/-} mice were treated with 1×10¹¹ particles of rAAV1-GAA in the quadriceps muscle. Wild-type (wt) and untreated Gaa^{-/-} (mock) mice were used as controls. Muscle tissues were isolated at 6 weeks after treatment and assayed for GAA activity. The bars represent mean±SEM GAA activity for three mice in each group. FIG. 3B shows representative sections of sections from free vector- (left) and gel-based vector-treated (right) Gaa^{-/-} mouse diaphragms, stained for glycogen using periodic acid-Schiff's reagent. Glycogen-containing vacuoles and regions acquire a pink stain using this technique.

FIG. 4 shows biodistribution of rAAV1 vector genomes after gel-based delivery. Nested PCR™ was used to amplify AAV genomes carrying the β-galactosidase gene after isolating tissues from gel-based rAAV1-βgal treated mice. Total cellular DNA was extracted and AAV genomes were amplified using primers specific for the βgal transgene. The expected product is 333 bp, and the positive control is the vector plasmid that was used to package the rAAV particles.

FIG. 5 is a graph showing conditional GAA expression in Mck-T-GAA/Gaa^{-/-} mice.

FIG. 6 is a graph showing GAA activity post intramyocardial injection of AAV.

FIG. 7 is a graph showing GAA activity after neonatal IV delivery.

FIG. 8 shows PAS of heart tissue.

FIG. 9 is a graph showing soleus contractile force.

FIG. 10 is a graph showing LacZ expression after neonatal intracardiac delivery.

### 4.0 DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

Mouse models of human disease provide invaluable opportunities to evaluate the potential efficacy of candidate therapies. Gene therapy strategies in particular have benefited enormously from the profusion of knockout and transgenic mice that recapitulate the genetic and pathophysiologic features of human diseases. Congenital myopathies, including the muscular dystrophies, have been widely investigated as targets for gene therapy interventions, and the diaphragm is often cited as one of the important target organs for functional correction (Petrof, 1998).

The mouse diaphragm presents unique challenges in terms of delivery of therapeutic agents due to its small size and thickness, which preclude direct injection into the muscle. Intravenous or intra-arterial delivery of vectors have not yet proven to be effective alternatives, but some studies are nevertheless currently under investigation (Baranov et al., 1999; Liu et al., 2001). However, isolation of blood vessels that specifically perfuse the diaphragm is also difficult in the mouse. Systemic delivery of vectors may eventually require the application of capsid-based targeting methods that have recently been reported (Buning et al., 2003; Muller et al., 2003; Perabo et al., 2003; Ponnazhagan et al., 2002; Shi et al., 2001; Shi and Bartlett, 2003; Wu et al., 2000).

### 4.1 Adeno-Associated Virus

Adeno-associated virus is a single-stranded DNA-containing, non-pathogenic human parvovirus that is being widely investigated as a therapeutic vector for a host of muscle disorders (Muzyczka, 1992; Kessler et al., 1996; Clark et al., 1997; Fisher et al., 1997). Six serotypes of the virus (AAV1-6) were originally described, and two more have recently been identified in rhesus macaques (Gao et al., 2002). Recombinant adeno-associated virus (rAAV) vectors have been developed in which the rep and cap open reading frames of the wild-type virus have been completely replaced by a therapeutic or reporter gene, retaining only the characteristic inverted terminal repeats (ITRs), the sole cis-acting elements required for virus packaging. Using helper plasmids expressing various combinations of the AAV2 rep and AAV1, 2, and 5 cap genes, respectively, efficient cross-packaging of AAV2 genomes into particles containing the AAV1, 2, or 5 capsid protein has been demonstrated (Grimm et al., 2003; Xiao et al., 1999; Zolotukhin et al., 2002; Rabinowitz et al., 2002). The various serotype vectors have demonstrated distinct tropisms for different tissue types in vivo, due in part to their putative cell surface receptors. Although several reports have indicated that rAAV1 vectors efficiently transduce skeletal muscle in general (Fraites et al., 2002; Chao et al., 2001; Hauck and Xiao, 2003), no study to date has reported which of the serotypes, if any, might transduce the diaphragm in particular.

### 4.2 Promoters and Enhancers

Recombinant vectors form important aspects of the present invention. The term “expression vector or construct” means any type of genetic construct containing a nucleic acid in which part or all of the nucleic acid encoding sequence is capable of being transcribed. In preferred embodiments, expression only includes transcription of the nucleic acid, for example, to generate a therapeutic polypeptide product from a transcribed gene that is comprised within one or more of the rAAV compositions disclosed herein.

Particularly useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively linked,” “operably linked,” “operatively positioned,” “under the control of” or “under the transcriptional control of” means that the promoter is in the correct location and orientation in relation to the nucleic acid segment that comprises the therapeutic gene to properly facilitate, control, or regulate RNA polymerase initiation and expression of the therapeutic gene to produce the therapeutic peptide, polypeptide, ribozyme, or antisense RNA molecule in the cells that comprise and express the genetic construct.

In preferred embodiments, it is contemplated that certain advantages will be gained by positioning the therapeutic agent-encoding polynucleotide segment under the control of one or more recombinant, or heterologous, promoter(s). As used herein, a recombinant or heterologous promoter is intended to refer to a promoter that is not normally associated with the particular therapeutic gene of interest in its natural environment. Such promoters may include promoters normally associated with other genes, and/or promoters isolated from any other bacterial, viral, eukaryotic, or mammalian cell.

Naturally, it will be important to employ a promoter that effectively directs the expression of the therapeutic agent-encoding nucleic acid segment in the cell type, organism, or even animal, chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high-level expression of the introduced DNA segment.

At least one module in a promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a mammalian, bacterial, fungal, or viral promoter. Exemplary such promoters include, for example, a 0-actin promoter, a native or modified CMV promoter, an AV or modified AV promoter, or an HSV or modified HSV promoter. In certain aspects of the invention, inducible promoters, such as tetracycline-controlled promoters, are also contemplated to be useful in certain cell types.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters that are well known in the art to achieve expression of a transgene is contemplated as well, provided that the levels of expression are sufficient for a given purpose. Tables 1 and 2 below list several elements/promoters that may be employed, in the context of the present invention, to regulate the expression of the therapeutic polypeptide-encoding rAAV constructs. This list is not intended to be exhaustive of all the possible elements involved in the promotion of transgene expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
PROMOTER AND ENHANCER ELEMENTS

| PROMOTER/ENHANCER | REFERENCES |
|---|---|
| Immunoglobulin Heavy Chain | Banerji et al., 1983; Gilles et al., 1983; Grosschedl and |
| | Baltimore, 1985; Atchinson and Perry, 1986, 1987; Imler |
| | et al., 1987; Weinberger et al., 1984; Kiledjian et al., |
| | 1988; Porton et al.; 1990 |
| Immunoglobulin Light Chain | Queen and Baltimore, 1983; Picard and Schaffner, 1984 |
| T-Cell Receptor | Luria et al., 1987; Winoto and Baltimore, 1989; Redondo |
| | et al.; 1990 |
| HLA DQ a and DQ β | Sullivan and Peterlin, 1987 |
| β-Interferon | Goodbourn et al., 1986; Fujita et al., 1987; Goodbourn |
| | and Maniatis, 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick and Benfield, 1989; Johnson |
| | et al., 1989 |
| Prealbumin (Transthyretin) | Costa et al., 1988 |
| Elastase I | Orntz et al., 1987 |
| Metallothionein | Karin et al., 1987; Culotta and Hamer, 1989 |
| Collagenase | Pinkert et al., 1987; Angel et al., 1987a |
| Albumin Gene | Pinkert et al., 1987; Tronche et al., 1989, 1990 |
| α-Fetoprotein | Godbout et al., 1988; Campere and Tilghman, 1989 |
| t-Globin | Bodine and Ley, 1987; Perez-Stable and Constantini, 1990 |
| β-Globin | Trudel and Constantini, 1987 |
| e-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α_{1-Antitrypain} | Latimer et al., 1990 |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh and |
| | Lockett, 1985; Firak and Subramanian, 1986; Herr and |
| | Clarke, 1986; Imbra and Karin, 1986; Kadesch and Berg, |
| | 1986; Wang and Calame, 1986; Ondek et al., 1987; Kuhl |
| | et al., 1987; Schaffner et al., 1988 |
| Polyoma | Swartzendruber and Lehman, 1975; Vasseur et al., 1980; |
| | Katinka et al., 1980, 1981; Tyndell et al., 1981; Dandolo |
| | et al., 1983; de Villiers et al., 1984; Hen et al., 1986; |
| | Satake et al., 1988; Campbell and Villarreal, 1988 |
| Retroviruses | Kriegler and Botchan, 1982, 1983; Levinson et al., 1982; |
| | Kriegler et al., 1983, 1984a, b, 1988; Bosze et al., 1986; |
| | Miksicek et al., 1986; Celander and Haseltine, 1987; |
| | Thiesen et al., 1988; Celander et al., 1988; Choi et al., |
| | 1988; Reisman and Rotter, 1989 |
| Papilloma Virus | Campo et al., 1983; Lusky et al., 1983; Spandidos and |
| | Wilkie, 1983; Spalholz et al., 1985; Lusky and Botchan, |
| | 1986; Cripe et al., 1987; Gloss et al., 1987; Hirochika |
| | et al., 1987; Stephens and Hentschel, 1987 |
| Hepatitis B Virus | Bulla and Siddiqui, 1986; Jameel and Siddiqui, 1986; |
| | Shaul and Ben-Levy, 1987; Spandau and Lee, 1988; |
| | Vannice and Levinson, 1988 |
| Human Immunodeficiency Virus | Muesing et al., 1987; Hauber and Cullan, 1988; |
| | Jakobovits et al., 1988; Feng and Holland, 1988; Takebe |
| | et al., 1988; Rosen et al., 1988; Berkhout et al., 1989; |
| | Laspia et al., 1989; Sharp and Marciniak, 1989; Braddock |
| | et al., 1989 |
| Cytomegalovirus | Weber et al., 1984; Boshart et al., 1985; Foecking and |
| | Hofstetter, 1986 |
| Gibbon Ape Leukemia Virus | Holbrook et al., 1987; Quinn et al., 1989 |

TABLE 2
INDUCIBLE ELEMENTS

| ELEMENT | INDUCER | REFERENCES |
|---|---|---|
| MT II | Phorbol Ester (TFA) | Palmiter et al., 1982; Haslinger |
| | Heavy metals | and Karin, 1985; Searle et al., |
| | | 1985; Stuart et al., 1985; |
| | | Imagawa et al., 1987, Karin et al., |
| | | 1987; Angel et al., 1987b; |
| | | McNeall et al., 1989 |
| MMTV (mouse mammary | Glucocorticoids | Huang et al., 1981; Lee et al., |
| tumor virus) | | 1981; Majors and Varmus, 1983; |
| | | Chandler et al., 1983; Lee et al., |
| | | 1984; Ponta et al., 1985; Sakai |
| | | et al., 1988 |
| β-Interferon | poly(rI)x | Tavernier et al., 1983 |
| | poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | Ela, SV40 Large T Antigen | Taylor et al., 1989; Taylor and |
| | | Kingston, 1990a, b |
| Proliferin | Phorbol Ester-TPA | Mordacq and Linzer, 1989 |
| Tumor Necrosis Factor | FMA | Hensel et al., 1989 |
| Thyroid Stimulating | Thyroid Hormone | Chatterjee et al., 1989 |
| Hormone a Gene | | |

As used herein, the terms “engineered” and “recombinant” cells are intended to refer to a cell into which an exogenous DNA segment, such as DNA segment that leads to the transcription of a therapeutic agent, such as a therapeutic peptide, polypeptide, ribozyme, or catalytic mRNA molecule has been introduced. Therefore, engineered cells are distinguishable from naturally occurring cells, which do not contain a recombinantly introduced exogenous DNA segment. Engineered cells are thus cells having DNA segment introduced through the hand of man.

To express a therapeutic gene in accordance with the present invention one would prepare an rAAV expression vector that comprises a therapeutic peptide-polypeptide-ribozyme- or antisense mRNA-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded polypeptide. This is the meaning of “recombinant expression” in this context. Particularly preferred recombinant vector constructs are those that comprise an rAAV vector comprised within the novel gel-based pharmaceutical vehicles disclosed herein. Such vectors are described in detail herein, and are also described in detail in U.S. Pat. Nos. 20 6,146,874, and 6,461,606; U.S. Pat. Appl. Publ. Nos. US2003/0095949, US2003/0082162; and PCT Intl. Pat. Appl. Publ. Nos. PCT/US99/11945, PCT/US99/21681, PCT/US98/08003, PCT/US98/07968, PCT/US99/08921, PCT/US99/22052, PCT/US00/11509, PCT/US02/13679, PCT/US03/13583, PCT/US03/13592, PCT/US03/08667, PCT/US03/20746, PCT/US03/12324, and PCT/US03/12225 (each of which is commonly owned with the present application, and is specifically incorporated herein by reference in its entirety).

### 4.3 Pharmaceutical Compositions

In certain embodiments, the present invention concerns formulation of one or more of the rAAV compositions disclosed herein in pharmaceutically acceptable solutions for administration to a cell or an animal, either alone, or in combination with one or more other modalities of therapy. In particular, the present invention contemplates the formulation of one or more rAAV vectors, virions, or virus particles (or pluralities thereof) using a water-soluble glycerin-based gel.

In such pharmaceutical compositions, it will also be understood that, if desired, the rAAV-encoded nucleic acid segment, RNA, DNA or PNA compositions that express one or more therapeutic gene product(s) as disclosed herein may be administered in combination with other agents as well, such as, e.g., peptides, proteins or polypeptides or various pharmaceutically-active agents, including one or more systemic or topical administrations of the gel-based rAAV vector formulations described herein. In fact, there is virtually no limit to other components that may also be included, given that the additional agents do not cause a significant adverse effect upon contact with the target cells or host tissues. The rAAV compositions may thus be delivered along with various other agents as required in the particular instance. Such compositions may be purified from host cells or other biological sources, or alternatively may be chemically synthesized as described herein. Likewise, such compositions may further comprise substituted or derivatized RNA, DNA, or PNA compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, topical, sublingual, subcutaneous, transdermal, parenteral, intravenous, intranasal, and intramuscular administration and formulation.

In typical application, the water-soluble glycerin-based gel formulations utilized in the preparation of pharmaceutical delivery vehicles that comprise one or more rAAV constructs may contain at least about 0.1% of the water-soluble glycerin compound or more, although the percentage of the active ingredient(s) may, of course, be varied and may conveniently be between about 1% and about 95% or more preferably, between about 5% and about 80%, and stil more preferably, between about 10% and about 60% or more of the weight or volume of the total pharmaceutical rAAV formulation, although the inventors contemplate any concentrations within those ranges may be useful in particular formulations. Naturally, the amount of the gel compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Owing to particular gel's characteristics, (from extremely viscous to almost water-like) the amount of gel used in the disclosed rAAV compositions may be titrated to achieve desirable or optimal results in particular treatment regimens. Such formulations, and the determination of the appropriate gel and concentration to use will be within the abilities of the artisan skilled in this field having benefit of the present teachings.

While the embodiments presented herein have specifically incorporated water-soluble glycerin gels, other gel compositions are also contemplated to be useful depending upon the particular embodiment, and as such are considered to fall within the scope of the present disclosure. For example, other currently commercially-available glycerin-based gels, glycerin-derived compounds, conjugated, or crosslinked gels, matrices, hydrogels, and polymers, as well as gelatins and their derivatives, alginates, and alginate-based gels, and even various native and synthetic hydrogel and hydrogel-derived compounds are all expected to be useful in the formulation of various rAAV pharmaceutical compositions. Specifically, illustrative embodiment gels include, but are not limited to, alginate hydrogels SAF-Gel (ConvaTec, Princeton, N.J.), Duoderm Hydroactive Gel (ConvaTec), Nu-gel (Johnson & Johnson Medical, Arlington, Tex.); Carrasyn (V) Acemannan Hydrogel (Carrington Laboratories, Inc., Irving, Tex.); glycerin gels Elta Hydrogel (Swiss-American Products, Inc., Dallas, Tex.) and K-Y Sterile (Johnson & Johnson). In addition, viscous contrast agents such as iodixanol (Visipaque, Amersham Health), and sucrose-based mediums like sucrose acetate isobutyrate (SAIB) (Eastman Chemical Company, Kingsport, Tenn.) are also contemplated to be useful in certain embodiments. Additionally, biodegradable biocompatible gels may also represent compounds present in certain of the rAAV formulations disclosed and described herein.

In certain embodiments, a single gel formulation may be used, in which one or more rAAV compositions may be present, while in other embodiments, it may be desirable to form a pharmaceutical composition that comprises a mixture of two or more distinct gel formulations may be used, in which one or more rAAV particles, virus, or virions are present. Various combinations of sols, gels and/or biocompatible matrices may also be employed to provide particularly desirable characteristics of certain viral formulations. In certain instances, the gel compositions may be cross-linked by one or more agents to alter or improve the properties of the virus/gel composition.

In certain circumstances it will be desirable to deliver the pharmaceutical compositions disclosed herein parenterally, intravenously, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety). Solutions of the active compounds as freebase or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a mammal, and in particular, when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified. In certain embodiments, the rAAV-gel compositions of the present invention may be formulated for topical, or transdermal delivery to one or more tissue sites or cell types within the body of the vertebrate being treated. Alternatively, in the embodiments where ex vivo or ex situ modalities are preferred, the rAAV-gel compositions of the invention my be used externally from the body of the intended recipient by first contacting a cell suspension or a tissue sample, or other extracorporeal composition with the rAAV-gel compositions to facilitate transfer of the rAAV vectors into the cells or tissues in ex vivo fashion. Following suitable transfection, then, such cells or tissues could be reintroduced into the body of the animal being treated.

### 4.4 Liposome-, Nanocapsule-, and Microparticle-Mediated Delivery

In certain embodiments, the rAAV-gel based compositions of the present invention may further comprise one or more liposomes, nanocapsules, microparticles, microspheres, lipid particles, vesicles, and the like, for enhancing, facilitating, or increasing the effectiveness of introducing the therapeutic rAAV compositions of the present invention into suitable host cells, tissues, or organs. In particular, the addition of a lipid particle, a liposome, a vesicle, a nanosphere, or a nanoparticle or the like to the gel-based compositions of the invention may serve to enhance or facilitate the delivery of the rAAV vectors, virions, or virus particles into the target cells or tissues.

Such formulations may be preferred for the introduction of pharmaceutically acceptable formulations of the nucleic acids or the rAAV constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1990a; Imaizumi et al., 1990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori and Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

In addition to the teachings of Couvreur et al. (1977; 1980), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

Alternatively, the invention provides for pharmaceutically acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; Couvreur, 1988; zur Muhlen et al., 1998; Zambaux et al. 1998; Pinto-Alphandry et al., 1995 and U.S. Pat. No. 5,145,684, specifically incorporated herein by reference in its entirety).

### 4.5 Therapeutic and Diagnostic Kits

The invention also encompasses one or more compositions together with one or more pharmaceutically-acceptable excipients, carriers, diluents, adjuvants, and/or other components, as may be employed in the formulation of particular rAAV-polynucleotide delivery formulations, and in the preparation of therapeutic agents for administration to a mammal, and in particularly, to a human, for one or more of the indications described herein for which rAAV-based gene therapy provides an alternative to current treatment modalities. In particular, such kits may comprise one or more gel-based rAAV composition in combination with instructions for using the viral vector in the treatment of such disorders in a mammal, and may typically further include containers prepared for convenient commercial packaging.

As such, preferred animals for administration of the pharmaceutical compositions disclosed herein include mammals, and particularly humans. Other preferred animals include murines, bovines, equines, porcines, canines, and felines. The composition may include partially or significantly purified rAAV compositions, either alone, or in combination with one or more additional active ingredients, which may be obtained from natural or recombinant sources, or which may be obtainable naturally or either chemically synthesized, or alternatively produced in vitro from recombinant host cells expressing DNA segments encoding such additional active ingredients.

Therapeutic kits may also be prepared that comprise at least one of the compositions disclosed herein and instructions for using the composition as a therapeutic agent. The container means for such kits may typically comprise at least one vial, test tube, flask, bottle, syringe or other container means, into which the disclosed water-soluble gel-based rAAV composition(s) may be placed, and preferably suitably aliquoted. Where a second therapeutic composition is also provided, the kit may also contain a second distinct container means into which this second composition may be placed. Alternatively, the plurality of therapeutic compositions may be prepared in a single pharmaceutical composition, and may be packaged in a single container means, such as a vial, flask, syringe, bottle, or other suitable single container means. The kits of the present invention will also typically include a means for containing the vial(s) in close confinement for commercial sale, such as, e.g., injection or blow-molded plastic containers into which the desired vial(s) are retained.

### 4.6 Methods of Nucleic Acid Delivery and DNA Transfection

In certain embodiments, it is contemplated that one or more of the rAAV-delivered therapeutic product-encoding RNA, DNA, PNAs and/or substituted polynucleotide compositions disclosed herein will be used to transfect an appropriate host cell. Technology for introduction of rAAVs comprising one or more PNAs, RNAs, and DNAs into target host cells is well known to those of skill in the art.

Several non-viral methods for the transfer of expression constructs into cultured mammalian cells also are contemplated by the present invention for use in certain in vitro embodiments, and under conditions where the use of rAAV-mediated delivery is less desirable. These include calcium phosphate precipitation (Graham and Van Der Eb, 1973; Chen and Okayama, 1987; Rippe et al., 1990) DEAE-dextran (Gopal, 1985), electroporation (Wong and Neumann, 1982; Fromm et al., 1985; Tur-Kaspa et al., 1986; Potter et al., 1984; Suzuki et al., 1998; Vanbever et al., 1998), direct microinjection (Capecchi, 1980; Harland and Weintraub, 1985), DNA-loaded liposomes (Nicolau and Sene, 1982; Fraley et al., 1979; Takakura, 1998) and lipofectamine-DNA complexes, cell sonication (Fechheimer et al., 1987), gene bombardment using high velocity microprojectiles (Yang et al., 1990; Klein et al., 1992), and receptor-mediated transfection (Curiel et al., 1991; Wagner et al., 1992; Wu and Wu, 1987; Wu and Wu, 1988). Some of these techniques may be successfully adapted for in vivo or ex vivo use.

### 4.7 Expression in Animal Cells

The inventors contemplate that a polynucleotide comprising a contiguous nucleic acid sequence that encodes a therapeutic agent of the present invention may be utilized to treat one or more cellular defects in a host cell that comprises the vector. Such cells are preferably animal cells, including mammalian cells such as those obtained from a human or other primates, murine, canine, feline, ovine, caprine, bovine, equine, epine, or porcine species. In particular, the use of such constructs for the treatment and/or amelioration of one or more diseases, dysfunctions, or disorders in a human subject that has, is suspected having, or has been diagnosed with such a condition is highly contemplated. The cells may be transformed with one or more rAAV gel-based vector compositions that comprise at least a first therapeutic construct of interest, such that the genetic construct introduced into and expressed in the host cells of the animal is sufficient to treat, alter, reduce, diminish, ameliorate or prevent one or more deleterious conditions in such an animal when the composition is administered to the animal either ex situ, in vitro and/or in vivo.

### 4.8 Transgenic Animals

It is contemplated that in some instances the genome of a transgenic non-human animal of the present invention will have been altered through the stable introduction of one or more of the rAAV-delivered polynucleotide compositions described herein, either native, synthetically modified, or mutated. As used herein, the term “transgenic animal” is intended to refer to an animal that has incorporated exogenous DNA sequences into its genome. In designing a heterologous gene for expression in animals, sequences which interfere with the efficacy of gene expression, such as polyadenylation signals, polymerase II termination sequences, hairpins, consensus splice sites and the like, are eliminated. Current advances in transgenic approaches and techniques have permitted the manipulation of a variety of animal genomes via gene addition, gene deletion, or gene modifications (Franz et al., 1997). For example, mosquitoes (Fallon, 1996), trout (Ono et al., 1997), zebrafish (Caldovic and Hackett, 1995), pigs (Van Cott et al., 1997) and cows (Haskell and Bowen, 1995), are just a few of the many animals being studied by transgenics. The creation of transgenic animals that express human proteins such as α-1-antitrypsin, in sheep (Carver et al., 1993); decay accelerating factor, in pigs (Cozzi et al., 1997), and plasminogen activator, in goats (Ebert et al., 1991) has previously been demonstrated. The transgenic synthesis of human hemoglobin (U.S. Pat. No. 5,602,306) and fibrinogen (U.S. Pat. No. 5,639,940) in non-human animals have also been disclosed, each specifically incorporated herein by reference in its entirety. Further, transgenic mice and rat models have recently been described as new directions to study and treat cardiovascular diseases such as hypertension in humans (Franz et al., 1997; Pinto-Siestma and Paul, 1997). The construction of a transgenic mouse model has recently been used to assay potential treatments for Alzheimer's disease (U.S. Pat. No. 5,720,936, specifically incorporated herein by reference in its entirety). It is contemplated in the present invention that transgenic animals contribute valuable information as models for studying the effects of rAAV-delivered therapeutic compositions on correcting genetic defects and treating a variety of disorders in an animal.

### 4.9 Site-Specific Mutagenesis

Site-specific mutagenesis is a technique useful in the preparation of individual peptides, or biologically functional equivalent polypeptides, through specific mutagenesis of the underlying polynucleotides that encode them. The technique, well-known to those of skill in the art, further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or change the properties of the polynucleotide itself, and/or alter the properties, activity, composition, stability, or primary sequence of the encoded polypeptide.

In certain embodiments of the present invention, the inventors contemplate the mutagenesis of the disclosed rAAV constructs to alter the activity or effectiveness of such constructs in increasing or altering their therapeutic activity, or to effect higher or more desirable introduction in a particular host cell or tissue. Likewise in certain embodiments, the inventors contemplate the mutagenesis of the therapeutic genes comprised in such rAAV vector themselves, or of the rAAV delivery vehicle to facilitate improved regulation of the particular therapeutic construct's activity, solubility, stability, expression, or efficacy in vitro, in situ, and/or in vivo.

The techniques of site-specific mutagenesis are well known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation that result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing. Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Another method for amplification is the ligase chain reaction (referred to as LCR), disclosed in Eur. Pat. Appl. Publ. No. 320,308 (specifically incorporated herein by reference in its entirety). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference in its entirety, describes an alternative method of amplification similar to LCR for binding probe pairs to a target sequence.

Qβ Replicase, described in PCT Intl. Pat. Appl. Publ. No. PCT/US87/00880, incorporated herein by reference in its entirety, may also be used as still another amplification method in the present invention. In this method, a replicative sequence of RNA that has a region complementary to that of a target is added to a sample in the presence of an RNA polymerase. The polymerase will copy the replicative sequence that can then be detected. An isothermal amplification method, in which restriction endonucleases and ligases are used to achieve the amplification of target molecules that contain nucleotide 5′-[α-thio]triphosphates in one strand of a restriction site (Walker et al., 1992), may also be useful in the amplification of nucleic acids in the present invention.

Strand Displacement Amplification (SDA) is another method of carrying out isothermal amplification of nucleic acids that involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA. Sequences can also be detected using a cyclic probe reaction (CPR). In CPR, a probe having a 3′ and 5′ sequences of non-target DNA and an internal or “middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods described in Great Britain Pat. Appl. No. 2 202 328, and in PCT Intl. Pat. Appl. Publ. No. PCT/US89/01025, each of which is incorporated herein by reference in its entirety, may be used in accordance with the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing a primer that has sequences specific to the target sequence. Following polymerization, DNA/RNA hybrids are digested with RNase H while double stranded DNA molecules are heat-denatured again. In either case the single stranded DNA is made fully double stranded by addition of second target-specific primer, followed by polymerization. The double stranded DNA molecules are then multiply transcribed by a polymerase such as T7 or SP6. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H (RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, incorporated herein by reference in its entirety, disclose a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “RACE” (Frohman, 1990), and “one-sided PCR” (Ohara et al., 1989) which are well-known to those of skill in the art. Methods based on ligation of two (or more) oligonucleotides in the presence of nucleic acid having the sequence of the resulting “di-oligonucleotide”, thereby amplifying the di-oligonucleotide (Wu and Dean, 1996, incorporated herein by reference in its entirety), may also be used in the amplification of DNA sequences of the present invention.

### 4.10 Biological Functional Equivalents

Modification and changes may be made in the structure of the rAAV vectors or the therapeutic agents encoded by the and still obtain functional vectors, viral particles, and virion that encode one or more therapeutic agents with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 3. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

TABLE 3

| | |
|---|---|
| AMINO ACIDS | CODONS |

| | | | |
|---|---|---|---|
| Alanine | Ala | A | GCA GCC GCG GCU |
| Cysteine | Cys | C | UGG UGU |
| Aspartic acid | Asp | D | GAG GAU |
| Glutamic acid | Glu | E | GAA GAG |
| Phenylalanine | Phe | F | UUG UUU |
| Glycine | Gly | G | GGA GGG GGG GGU |
| Histidine | His | H | GAG CAU |
| Isoleucine | Ile | I | AUA AUG AUU |
| Lysine | Lys | K | AAA AAG |
| Leucine | Leu | L | UUA UUG GUA GUG GUG GUU |
| Methionine | Met | M | AUG |
| Asparagine | Asn | N | AAG AAU |
| Proline | Pro | P | GGA GGG GGG GGU |
| Glutamine | Gln | Q | GAA GAG |
| Arginine | Arg | R | AGA AGG GGA GGG GGG GGU |
| Serine | Ser | S | AGG AGU UGA UGG UGG UGU |
| Threonine | Thr | T | AGA AGC AGG AGU |
| Valine | Val | V | GUA GUG GUG GUU |
| Tryptophan | Trp | W | UGG |
| Tyrosine | Tyr | Y | UAG UAU |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, ie. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred. As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

### 4.11 Glycogen Storage Disease Type II (GSDII) Pompe's Disease)

GSDII is an inherited disorder of glycogen metabolism, resulting from a lack of functional acid α-glucosidase (GAA), and is characterized by progressive skeletal muscle weakness (Hers, 1963; Hirschhorn and Reuser, 2000). GAA is responsible for cleaving α-1,4 and α-1,6 linkages of lysosomal glycogen, which leads to the release of monosaccharides (Hirschhorn and Reuser, 2000; Baudhuin and Hers, 1964). A deficiency of functional GAA results in massive accumulation of glycogen in lysosomal compartments of striated muscle, resulting in disruption of the contractile machinery of the cell. Affected individuals begin storing glycogen in utero, ultimately resulting in a variety of pathophysiological effects, most significantly of which are severe cardiomyopathy and respiratory insufficiency (Moufarrej and Bertorini, 1993). Clinical presentation of GSDII disease can occur within the first few months of life, and most affected infants do not survive past two years of age due to cardio-respiratory failure (Hers, 1963; Hirschhorn and Reuser, 2000; Reuser et al., 1995). There are no currently established treatments for GSDII disease, however enzyme replacement therapy is being tested in clinical trials.

Strict genotype-phenotype correlations have not been established due to the small population of patients and the observation that some patients with identical mutations in the GAA gene have markedly different clinical presentations (Anand, 2003). The existence of modifier genes has been proposed, but to-date none have been identified.

### 4.12 Recombinant AAV-Mediated Gene Therapy

Recombinant AAV-based gene therapy vectors are at the forefront of viral vector-based human gene therapy applications and are currently being assessed in clinical trials (Manno et al., 2003; Wagner et al., 2002). Advantages of rAAV vectors include the lack of any known pathologies associated with AAV infection, the ability to infect non-dividing cells, the lack of any viral genes in the vector, and the ability to persist long-term in infected cells (Berns and Linden, 1995; Berns and Giraud, 1996; Mah et al., 2002; Muzyczka, 1992; Rabinowitz and Samulski, 1998). To-date, over 40 different clones of AAV have been isolated, of which serotypes 1 though 9 have been developed into gene therapy vectors (Gao et al., 2003; Gao et al., 2002). Recently, several studies have demonstrated alternate tissue tropisms for each AAV serotype (Chao et al., 2000; Fraites et al., 2003; Rutledge et al., 1998; Zabner et al., 2000).

Recombinant AAV-mediated gene therapy strategies have demonstrated significant promise for the treatment of GSDII and the muscular dystrophies. Preclinical studies have demonstrated phenotypic correction of a mouse model of GSDII using rAAV2 and rAAV1 vectors, with up to eight-fold over-expression of functional Gaa in the treated tissues (Fraites et al., 2003; Fraites et al., 2002; Mah et al., 2004).

### 4.13 Cardiac Gene Transfer

Only recently has myocardium become a main target of rAAV-mediated gene transfer. Similar to intramuscular administration, a hurdle for efficient cardiac gene transfer is achieving widespread distribution of vector throughout the affected tissue. Most studies to date have implemented direct cardiac injection of vector, which have led to efficient transduction localized around the site of injection Champion et al., 2003; Chu et al., 2004; Li et al., 2003; Yue et al., 2003). Fraites et al. (2002) was able to demonstrate near-normal levels of cardiac GAA activity in a mouse model of GSDII via direct cardiac injection of a rAAV2-based vector. Methods to further distribute vector transduction include induction of temporary cardiac ischemia followed by perfusion of vector, ex vivo infusion followed by transplantation, and the co-administration of vector with cardioplegic substances (Gregorevic et al., 2004; Iwatate et al., 2003).

### 4.14 Gene Transfer to Diaphragm

Due to its small size and thickness, the mouse diaphragm presents distinct challenges for the delivery of therapeutic agents. Previous diaphragm-targeted delivery methods have included via intravenous injection followed by transient occlusion of the vena cava and direct injection to the diaphragms of mice (Liu et al., 2001; Petrof et al., 1995; Stedman et al., 1991; Yang et al., 1998). Matrix-mediated vector delivery methods have been used extensively for gene therapy applications, particularly for non-viral gene delivery.

### 4.15 Neonatal Gene Transfer

The therapeutic paradigm for most progressive diseases is that the younger the age at treatment, the higher the likelihood for therapeutic success. This is may be in part due to the minimal progression of disease phenotype and the potential to avoid immune response to the vector and/or transgene product. Several studies have examined the potential for treatment at early age timepoints with neonatal and even in utero gene therapy (Bouchard et al., 2003). Rucker et al. (2004) demonstrated rAAV-mediated expression of GAA in a mouse model of GSDII after in utero administration. Although intraperitoneal delivery is effective in fetuses and neonates, its efficiency diminishes as the animals grow.

A recent study by Yue et al. demonstrated persistent cardiac transduction after direct injection of rAAV5 vectors into one-day-old mouse neonates (Yue et al., 2003). Transduction events clustered mainly in the inner and outer myocardium, with some intermittent positively transduced cells in the middle layer. Several groups have also shown successful liver transduction after intravenous injection of rAAV vectors into neonatal mice (Daly et al., 2001; Mah et al., 2003). This example demonstrates that intravenous administration of alternate serotypes of rAAV vectors can achieve high levels of cardiac transduction.

### 5.0 EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 5.1 Example 1

### Methods and Compositions for rAAV Vector Delivery to Diaphragm Muscle

The present example provides a safe, effective, and uniform method for delivery of recombinant adeno-associated virus vectors to the mouse diaphragm to facilitate gene therapy. The ability of rAAV serotypes 1, 2, and 5 to transduce the mouse diaphragm has been evaluated, and this example describes the application of a gel-based delivery method and demonstrates its utility for delivery of rAAV1, 2, and 5 to the mouse diaphragm. These results are the first to demonstrate efficient, uniform expression of a transgene in the murine diaphragm using rAAV vectors. Finally, the utility of this method was assessed using a mouse model (Gaa^{-/-}) of glycogen storage disease type II (GSDII) (Raben et al., 1998), an autosomal recessive disorder that is characterized by respiratory insufficiency secondary to diaphragmatic weakness in affected juveniles (Moufarrej and Bertorini, 1993).

5.1.1 Materials and Methods

5.1.1.1 Packaging and Purification of Recombinant AAV1, 2, and 5 Vectors

The recombinant AAV2 plasmids pAAV-lacZ (Kessler et al., 1996) and p43.2-GAA (Fraites et al., 2002) have been described previously. Recombinant AAV vectors were generated, purified, and titered at the University of Florida Powell Gene Therapy Center Vector Core Lab as previously described (Zolotukhin et al., 2002). Recombinant AAV particles based on serotypes 1, 2, and 5 were produced using pAAV-lacZ, whereas only rAAV1 particles (rAAV1-GAA) were packaged with p43.2-GAA.

5.1.1.2 Vector/Vehicle Preparation

A sterile, bacteriostatic, water-soluble, glycerin-based gel was used as a vehicle for vector application to the diaphragm (K-Y® Sterile, Johnson & Johnson Medical, Arlington, Tex.). Individual doses of virus were diluted in sterile phosphate buffered saline (PBS) for a total volume of 10 μl and then added to 150 μl of gel in a 2 ml microcentrifuge tube. The virus-vehicle suspension was vortexed for one minute and then centrifuged for one minute at maximum speed. Free virus was diluted in sterile PBS for a total volume of 50 μl.

5.1.1.3 In Vivo Delivery

All animal studies were performed in accordance with the guidelines of the University of Florida Institutional Animal Care and Use Committee. Adult 129X1×C57BL/6 (wild type) or Gaa^{-/-} mice (Raben et al., 1998) were anesthetized using 2% isoflurane and restrained supine on a warmed operating surface. In a sterile field, after reaching a surgical plane of anesthesia, a midline incision was made through the skin extending from the xyphoid process to the suprapubic region. An incision was made through the abdominal wall following the linea alba. The abdominal walls were retracted laterally, the gall bladder was carefully separated from the rib cage, and the liver was carefully retracted from the diaphragm using sterile cotton swabs.

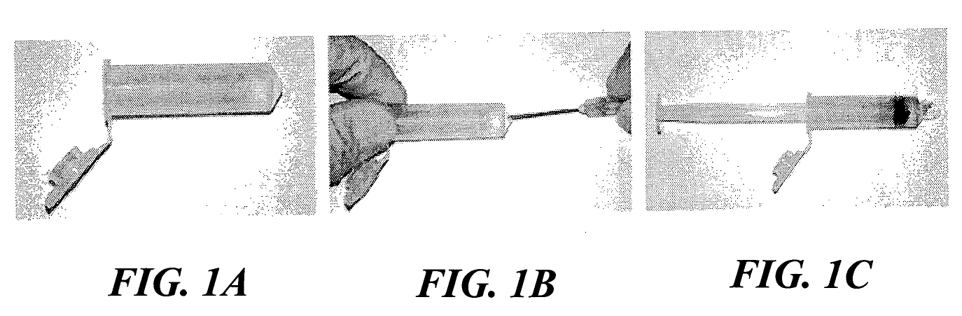

While lifting the xyphoid, free virus or virus mixed with vehicle were applied directly to the abdominal surface of the diaphragm. Free virus was applied using a pipet. To facilitate application of the gel to the diaphragm, a 22-gauge needle was used to puncture the bottom of the microcentrifuge tube and a plunger from a 3 cc syringe was used to force the gel through the hole and onto the diaphragm surface (FIG. 1). In some cases, a cotton-tipped applicator was used to ensure even spread over the entire diaphragm. After five min, the abdominal muscles were sutured and the skin was closed. Subcutaneous ampicillin (20-100 mg/kg) and buprenorphine (0.1 mg/kg) were administered prior to removing the animal from anesthesia.

5.1.1.4 Assays of β-Galactosidase and GAA Enzymatic Activity

Six weeks after the surgical procedure and gene delivery, tissue lysates were assayed for enzyme activity using the Galacto-Star chemiluminescent reporter gene assay system (Tropix Inc., Bedford, Mass.). Protein concentrations for tissue lysates were determined using the Bio-Rad DC protein assay kit (Bio-Rad, Hercules, Calif.). For rAAV1-GAA treated animals, enzymatic activity assays for GAA were performed six weeks after vector delivery as described previously (Fraites et al., 2002). Tissue homogenates were assayed for GAA activity by measuring the cleavage of the synthetic substrate 4-methyl-umbelliferyl-α-D-glucoside (Sigma M9766, Sigma-Aldrich, St. Louis, Mo.) after incubation for 1 h at 37° C. Successful cleavage yielded a fluorescent product that emits at 448 nm, as measured with an FLx800 microplate fluorescence reader (Bio-Tek Instruments, Winooski, Vt.). Protein concentration was measured as described above. Data are represented as nanomoles of substrate cleaved in one hour per milligram of total protein in the lysate (nmol/hr/mg).

5.1.1.5 Histological Assessment of Glycogen Clearance

Segments of treated and untreated diaphragm were fexed overnight in 2% glutaraldehyde in PBS, embedded in Epon, sectioned, and stained with periodic acid-Schiff (PAS) by standard methods (Raben et al., 1998).

5.1.1.6 Biodistribution of Vector Genomes

Tissues were removed using sterile instruments and snap-frozen in liquid nitrogen. Total cellular DNA was extracted from tissue homogenates using a Qiagen DNeasy® kit per the manufacturer's instructions (Qiagen, Valencia, Calif.). Nested PCR™ reactions were performed as follows: 1.5 !g total DNA was used as a template for the initial PCR™ amplification using the sense primer 5′-AGCTGGCGTAATAGCGAAGA-3′ (SEQ. ID NO:1) and reverse primer 5′-CGCGTCTCTCCAGGTAGCGAA-3′ (SEQ. ID NO:2), yielding a 1486-bp product. The PCR™ product was purified using the Qiagen MinElute PCR™ purification kit per the manufacturer's instructions, followed by PCR™ amplification using the sense primer 5′-CGGTGATGGTGCTGCGTTGGAG-3′ (SEQ. ID NO:3) and reverse primer 5′-TCGACGTTCAGACGTAGTGT-3′ (SEQ. ID NO:4), resulting in a final product of 333 bp. All reactions were performed under the following conditions: hot start denaturation at 94° C. for five min, followed by 30 cycles of denaturation at 94° C. for 1 min, annealing at 62° C. for 1 min, and extension at 72° C. for 2 min. Products were electrophoresed and analyzed using a 2% agarose gel.

5.1.2 Results

5.1.2.1 Efficiency of Transduction Using Gel-Based Delivery of rAAV in Vivo

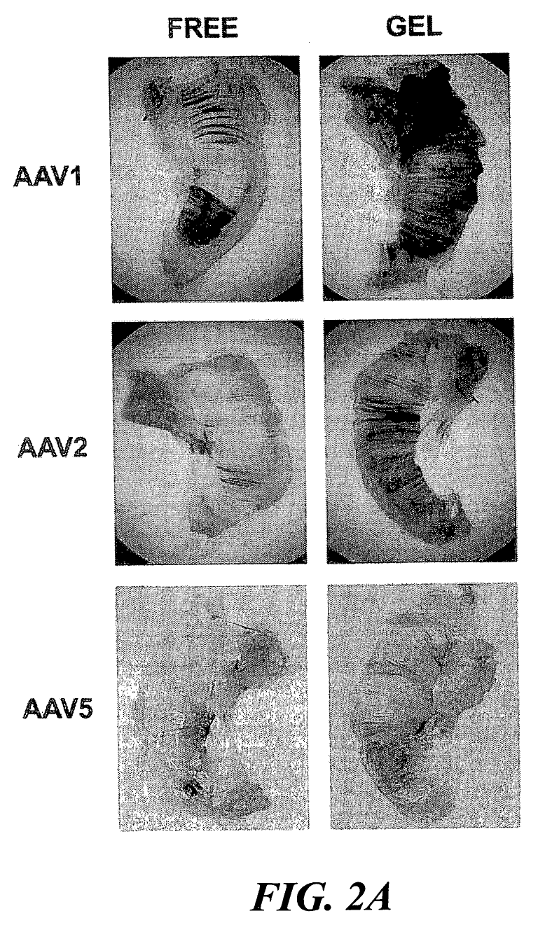

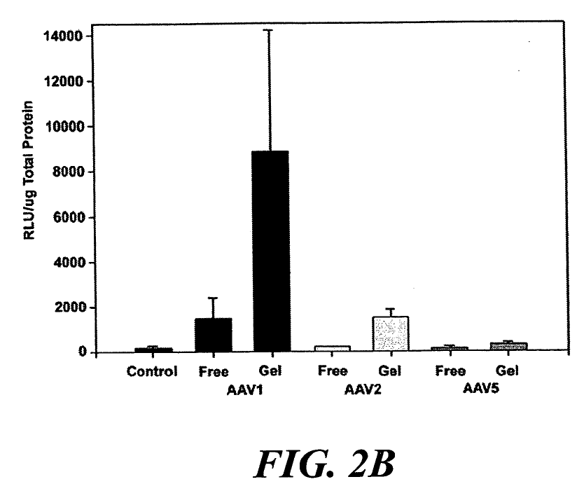

The efficiency of rAAV delivery using the gel-based method was compared to free virus delivery using β-galactosidase as a reporter gene (FIG. 2A). Direct particle-to-particle comparisons of histochemistry from free-virus-treated animals (left column) versus gel-based delivery (right column) indicate an increased efficiency of transduction for all serotypes using the latter method. Quantitative analysis of tissue lysates from these animals using the Galacto-Star enzymatic assay for β-galactosidase confirms these results (FIG. 2B). Activities for subjects treated with gel-vector suspensions had higher activities for all three serotypes.

5.1.2.2 Varying Tropisms of rAAV Serotypes 1,2, and 5 for Diaphragm Muscle in Vivo

The results from FIG. 2A and FIG. 2B also indicate a distinct gradient of tropism for mouse diaphragm among the three tested serotypes. Qualitatively, rAAV1 vectors led to the most intense staining under both the free virus and gel-based conditions. Differences between rAAV2 and rAAV5 were hard to distinguish in the free virus case due to the low levels of transduction for both vectors, but the gel-mediated subjects demonstrated a clear preference for rAAV2 compared to rAAV5. These results are further verified in FIG. 2B, which indicates higher levels of enzyme activity for rAAV2 gel suspensions compared to rAAV5. Taken together, the results of histochemical staining and enzymatic activity indicate: (1) a substantial increase in viral transduction using a physical delivery system; and (2) a clearly enhanced mouse diaphragm tropism for rAAV1, and a potentially important difference between rAAV2 and rAAV5.

5.1.2.3 Gel-Based Delivery of rAAV1-GAA Results in Biochemical Correction of Diaphragms in GAA^{-/-} Mice.

Having demonstrated increased transduction of the mouse diaphragm using the gel-based method, the ability of this method to restore enzymatic activity in a mouse model of glycogen storage disease type II (GSDII; MIM 232300), a lysosomal glycogen storage disease caused by a lack or deficiency of the lysosomal enzyme, acid α-glucosidase (GAA; EC 3.2.1.20) was assessed. The mouse model of this disease stores glycogen in all tissues, with significant pathologies in the heart and skeletal muscle (Raben et al., 1998). The use of rAAV vectors to restore enzymatic and functional activity in skeletal and cardiac muscle in these mice was previously characterized (Fraites et al., 2002). Coupled with new findings using a gel-based delivery method, it was hypothesized that gel-based delivery of rAAV1-GAA would be able to restore GAA activity in Gaa^{-/-} diaphragms and, in turn, reverse lysosomal glycogen accumulation.

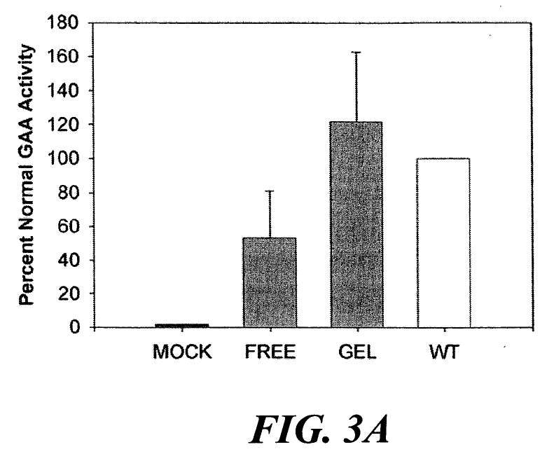

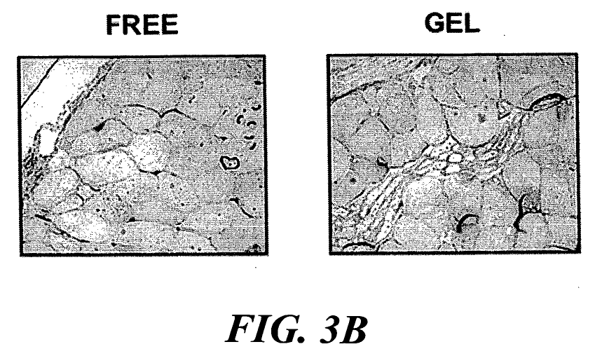

Using rAAV1-GAA vectors, increases in diaphragmatic transduction in Gaa^{-/-} mice similar to those seen in control mice with β-galactosidase vector were found. GAA enzymatic activities were restored to 50% of wild type with free vector, and were further increased to 120% of normal levels using a vector-gel suspension (FIG. 3A). These activities had a profound effect in glycogen storage, as assessed by periodic acid-Schiff's reagent (PAS) staining (FIG. 3B). Dark pink vacuoles, indicative of stored glycogen, are observed in free-vector-treated diaphragms from Gaa^{-/-} mice whereas a near-complete reversal of glycogen accumulation from diaphragms is seen in gel-treated mice.

5.1.2.4 Biodistribution of rAAV Genomes After Gel-Based Delivery

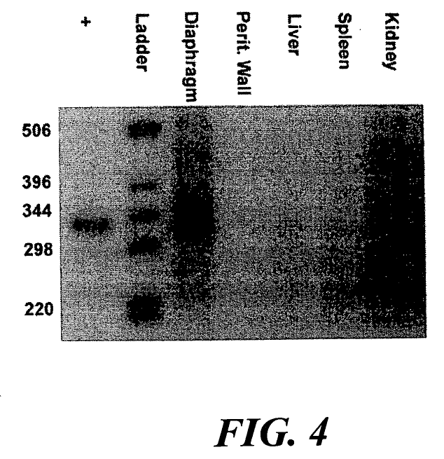

Since a secondary advantage of physical delivery systems may be the ability to restrict viral spread, it was also sought to determine which tissues endocytosed the viral vectors after gel-based delivery. To this end, various tissues from rAAV1-βgal gel-treated mice were harvested and total cellular DNA was extracted. Using a nested PCR™ technique, a portion of the β-galactosidase gene was amplified from vector genomes (FIG. 4). As expected, vector genomes could be detected in treated diaphragms. Vector genomes could not be detected in any other tissue examined (including sections of the peritoneal wall and liver adjacent to the diaphragm); however, it is possible that more sensitive detection methods (such as real-time PCR™) would detect trace amounts of vector genomes.

5.1.3 Discussion

Transduction events for recombinant adeno-associated viruses can be separated into five general stages: (1) binding and entry (endocytosis); (2) endosomal processing and escape; (3) transcytosis; (4) nuclear import and uncoating; and (5) genome conversion, including second-strand synthesis (or alternatively self-complementation), followed by genome concatemerization and/or integration into the host chromosome. This example describes, for the first time, an improvement in the process whereby enhancement of the first step of this process using a physical method prolongs viral dwell time and increases the efficiency of transduction by providing longer viral particle exposure times to receptors on target tissues.

Carrier molecules and delivery agents have been used extensively for gene therapy applications, particularly for non-viral gene delivery. With regard to viral vectors, recombinant adenoviruses have been used in concert with a variety of agents in order to increase or prolong bioavailability, thereby enhancing the efficiency of delivery. March et al. (1995) reported the use of poloxamer 407, a hydrogel which exhibits potentially useful, thermo-reversible gelation, enabling formulation at low temperature with subsequent hardening to a robust gel at room and physiologic temperatures. They demonstrated increased transduction of vascular smooth muscle cells in vitro, with similar findings reported in vivo by Van Belle et al. (1998). Unfortunately, poloxamers have recently been shown to have adverse effects on adeno-associated virus stability (Croyle et al., 2001). Likewise, thixotropic solutions have also shown promise for enhancing adenovirus-mediated transduction of airway epithelia (Seiler et al., 2002). Several other promising agents have also been effectively used with adenovirus vectors, including β-cyclodextrins, surfactants, and collagen- or gelatin-based matrices.

While extensive testing of potential adenovirus formulations has been reported, few similar studies are extant for adeno-associated viruses. Most of the available literature describes formulations that increase stability for storage or purification, but few reports address the need for augmented physical delivery of viral particles in vivo. These inventors and collaborators have previously described the use of microsphere-conjugated rAAV for systemic delivery of viral vectors, in which it was possible to significantly increase the transduction efficiency in target tissue beds in vivo by increasing vector dwell time (Mah et al., 2002). Similarly, a number of groups are currently developing capsid-modified rAAV vectors to target specific vascular beds upon systemic delivery. To date, however, the literature is devoid of other examples of physical delivery agents or methods to improve rAAV delivery to tissue surfaces, such as skin, blood vessel adventitia, or diaphragm.

The methods described herein for diaphragmatic delivery of rAAV vector-based composition reliy on retention of vector on the peritoneal surface of the diaphragm. Local delivery using this strategy is clinically achievable by endoscopic delivery and has the added benefit of reduced risk associated with systemic vascular delivery. While this method has been specifically applied to the murine diaphragm, the inventors believe that such gel-based AAV compositions have broad utility for improving the transduction efficiency of the vectors in a variety of tissues. (One important use contemplated by the inventors is that of topical application of gel-based rAAV compositions formulated for wound or burn healing).

Comparisons of rAAV serotype tropisms for skeletal muscle have already been reported (Fraites et al., 2002; Chao et al., 2001; Chao et al., 2000; Hauck and Xiao, 2003). Several recombinant AAV vectors based on alternative serotypes have demonstrated greater transduction efficiencies in skeletal muscle than serotype 2. In particular, several reports have shown nearly one log greater expression of a variety of transgenes when packaged in rAAV1 capsids compared to rAAV2. Similar findings have been reported with rAAV6, although this serotype has not been as widely studied (Hauck and Xiao, 2003; Moufarrej and Bertorini, 1993). Clear differences in serotype tropism were observed between rAAV1 and the other two serotypes in the context of gel-based delivery and free virus administration, with significant differences observed between rAAV1 and rAAV5 (p<0.1). The eight-fold over-expression of GAA in Gaa-deficient diaphragms after delivery of free rAAV1-GAA compared to serotype 2 (FIG. 2B, AAV1 Free vs. AAV2 Free) is nearly identical to prior observations after direct intramuscular administration of the same two vectors in tibialis anterior muscles of Gaa^{-/-} mice (Fraites et al., 2002), indicating a conserved rAAV1 tropism for skeletal muscle.

### 5.2 Example 2

### Murine Models of Glycogen Storage Disease Type II

For these studies, two different mouse models of GSDII are employed. For the gene therapy studies, a knockout mouse model of GSDII (Gaa^{-/-}) developed by Raben et al. is used. This mouse model was generated by the insertion of a neomycin gene cassette into exon 6 of the murine Gaa gene and recapitulates the human disease in that there is progressive skeletal muscle weakening and glycogen storage (Raben et al., 1998).

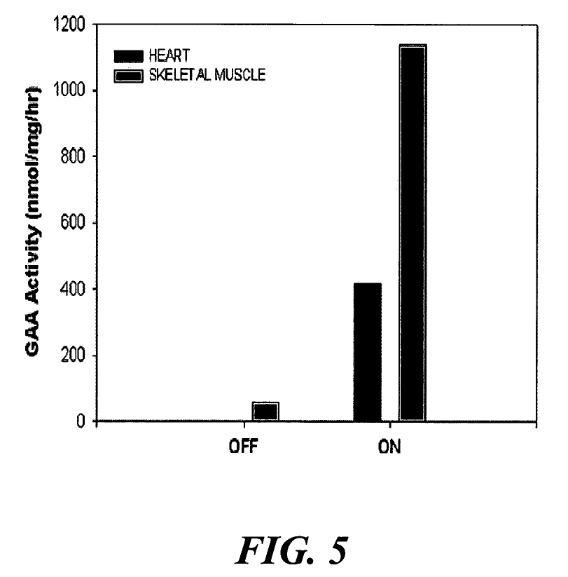

An alternative mouse model of GSDII (Mck-T-GAA/Gaa^{-/-}) in which human GAA can be conditionally-expressed in skeletal muscle in response to tetracycline in the context of the Gaa^{-/-} background is also used (Gossen and Bujard, 1992; Raben et al., 2001). GAA expression can be completely shut off when the animals are fed doxycycline (a tetracycline derivative)-supplemented food (FIG. 5). Raben et al. (2002) showed that glycogen clearance in Mck-T-GAA/Gaa^{-/-}mice could be achieved with modest levels of cardiac GAA expression in young animals, whereas, in older adult animals, supraphysiologic levels lead to only 40-50% glycogen clearance. Conversely, in skeletal muscle, greater than 8-fold normal levels of GAA activity were required to achieve complete clearance of glycogen in young animals. Using this conditionally-expressing model, the relationship of the severity of disease phenotype, or the stage of disease progression, may be characterized with the propensity for biochemical and functional correction. While clearance of glycogen is a crucial aspect in the successful treatment of GSDII, it is possible that a reduction, rather than complete clearance, of glycogen in affected tissues may result in significantly improved muscle function.

5.2.1 Recombinant AAV-Mediated Transduction in Adult Mouse Heart and Diaphragm

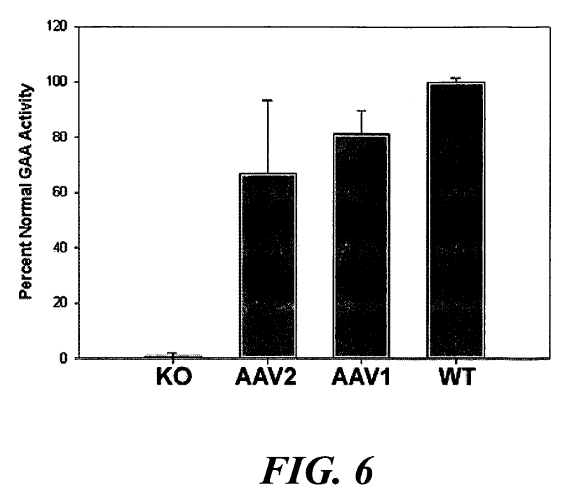

A study in which either rAAV1 or rAAV2 vector was administered via direct cardiac injection to adult Gaa^{-/-} mice resulted in near-normal levels of cardiac GAA activity with both serotypes (FIG. 6). Interestingly, when administered intravenously in neonate mice, there was a dramatic difference in cardiac GAA levels between the two serotypes. These results are further discussed below.

As the diaphragm is severely affected in GSDII and many other muscular dystrophies, the inventors sought to develop a new method of rAAV vector delivery to enhance diaphragm transduction. A gel biopolymer formulation was used to deliver 1×10¹¹ particles of rAAV-CMV-lacZ to adult 129X1×C57BL/6 mouse diaphragms. As shown in FIG. 2A, gross histochemical comparison of lacZ expression indicates an increased efficiency of transduction for all rAAV serotypes delivered in the gel. Quantitative enzyme detection analysis further confirmed this observation. Furthermore, it was also observed that rAAV serotype 1 vectors transduced diaphragm more efficiently than rAAV2- and 5-based vectors, whether delivered free or in gel vehicle.

The potential utility of matrix-mediated delivery of rAAV in a mouse model of GSDII was also investigated. 1×10¹¹ particles of therapeutic rAAV1 encoding the CMV promoter driven-human GAA gene (rAAV1-CMV-GAA) was administered directly to the diaphragm either in free or gel-based formulations. GAA enzymatic activities were restored to 50% of wild-type with free vector, and were further increased to 120% of normal levels using the vector-gel suspension. Furthermore, the high levels of GAA expression had a profound effect on glycogen storage, as assessed by periodic acid-Schiff's (PAS) reagent staining. As shown in FIG. 3B, stored glycogen (indicated by dark-stained vacuoles) is observed in free vector treated diaphragms, whereas a substantial reversal of glycogen accumulation is seen in diaphragms of gel-treated mice. In sum, these studies demonstrate the use of matrix-mediated delivery of rAAV vector to diaphragm for the treatment of skeletal myopathies.

5.2.2 Recombinant AAV-Mediated Transduction in Mouse Neonates

Although diaphragmatic transduction posed a technical challenge in adult mouse models, transduction of mouse neonate diaphragms has been much simpler. In initial studies, simple intraperitoneal injection of 1×10¹¹ particles rAAV2-CMV-lacZ vector at one-day of age resulted in almost complete transduction of the diaphragm, as assessed by X-gal staining four weeks post-injection.

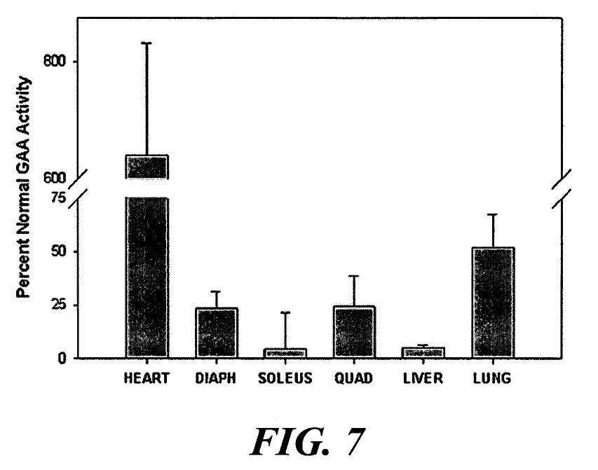

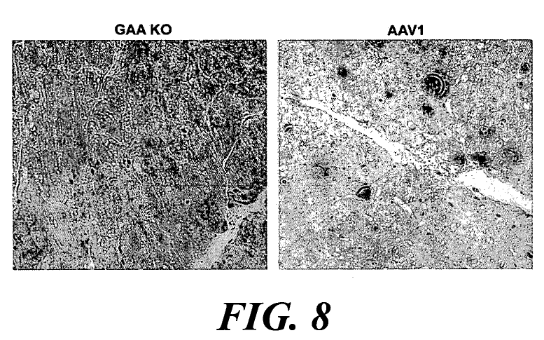

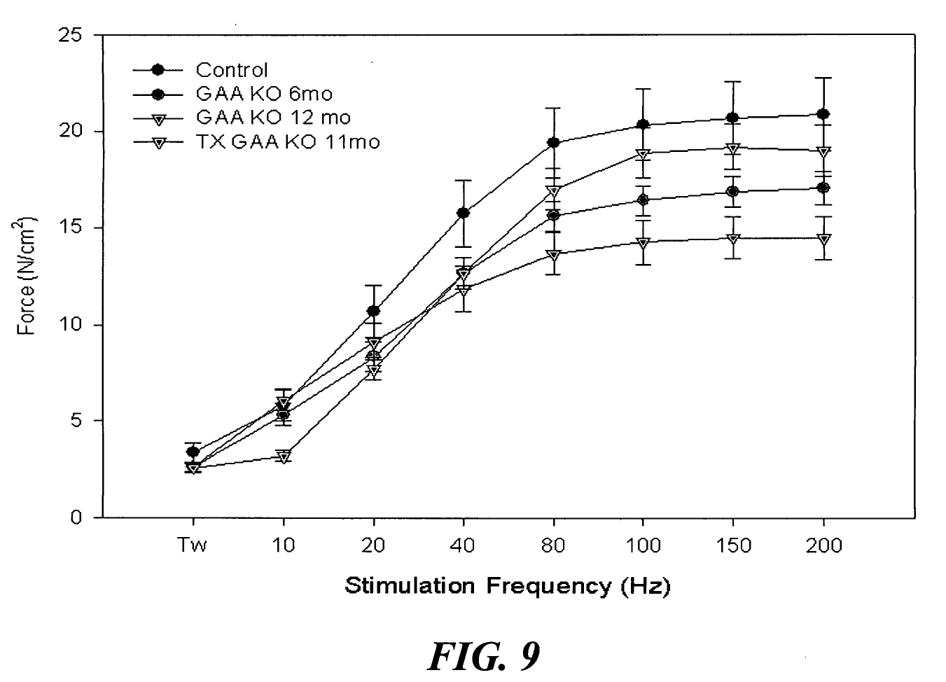

As described above, direct cardiac administration of rAAV1 and rAAV2 vectors to adult animals leads to normal levels of cardiac GAA activity. Studies were performed in which rAAV2 vectors encoding for CMV-GAA were administered intravenously to one-day-old Gaa^{-/-} mice. Similar to the adult animal studies, intravenous administration of rAAV2 to neonates also resulted in near-normal levels of cardiac GAA activity. Conversely, intravenous administration of 5×10¹⁰ particles of an rAAV1 vector in neonates resulted in supraphysiologic levels of GAA expression in the hearts of treated animals, with an average of 650% of normal levels, eleven months post-injection. In addition, levels of diaphragm, lung, and quadriceps GAA enzyme activity levels were above the therapeutic threshold of 20% (FIG. 7). As shown in FIG. 8, almost complete clearance of stored glycogen was observed in the hearts of treated animals, as determined by PAS staining of heart sections. Biodistribution analysis of vector genomes in treated animals suggested that the high levels of heart and diaphragm GAA activity were a result of rAAV-mediated transduction of those tissues, with studies suggesting approximately 0.42 vector genomes per diploid cell in transduced heart. Despite high levels of GAA activity in the lung, no significant transduction of the lung was noted, as determined by extremely low vector genome copies and a lack of transgene specific RT-PCR product, suggesting that the heart is secreting expressed enzyme, which in turn is taken up by the lung. Access to the bloodstream and the ability to promote systemic circulation of secreted proteins makes the heart as a depot organ, in which therapeutic enzyme can be produced and secreted, an interesting concept. As shown in FIG. 9, a marked improvement was noted in soleus muscle function in treated mice as compared to age-matched control animals and even animals five months younger in age. Although significant functional improvement was noted in soleus muscle, the levels of GAA activity were not above the 20% therapeutic threshold. Several potential explanations include that only minimal levels of GAA activity are required for functional correction of the soleus muscle, the soleus had substantial GAA activity at an earlier timepoint protecting it from severe muscle pathology, or the improved heart and diaphragm function resulted in overall better circulation and general health which was to some extent protective. These results demonstrate the use of intravenous administration of alternate rAAV serotype vectors to transduce multiple target tissues.

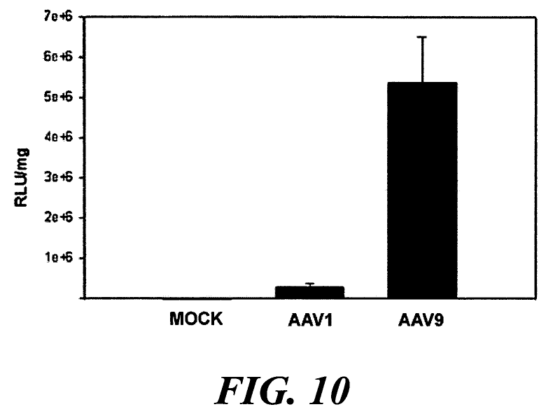

Recent data suggests that other serotypes may be even more efficient at globally transducing skeletal and cardiac muscle than rAAV1 vectors. Another AAV serotype, serotype 9, has been developed as a gene therapy vector (Limberis et al., 2004; Wang et al., 2004). As shown in FIG. 10, direct cardiac administration of rAAV1 or rAAV9 vectors encoding for CMV-lacZ to neonatal mice resulted in substantially higher levels of transgene expression from the rAAV9 vector than the rAAV1 vector, four-weeks post-injection. These results suggest that rAAV9-based vectors may be more effective vectors for cardiac-targeted gene transfer.

5.2.3 Gene Expression Profiling of GSDII

The potential for modifying genes to be involved in the pathology of GSDII has been proposed, though to-date, none have been clearly identified. Studies have been performed in an attempt to identify such modifying genes. GAA-deficient myoblasts isolated from Gaa^{-/-} mice or from GSDII patient samples were transduced with rAAV1-CMV-GAA or control vector. RNA was isolated from the cells and processed and analyzed on Affymetrix Murine Genome U74Av2 or Human Genome U133A Plus 2.0 GeneChips. The geometric mean hybridization intensities were analyzed to identify genes that differentiated among the three treatment classes: mock infection, rAAV1-factor VIII (FVIII) infection (control), and rAAV1-GAA infection. Of the 7676 genes considered in the murine myoblast analysis, 53 genes differentiated among the treatment classes and could function as classifiers of treatment response (P≦0.001). Of the 53 identifying genes, five genes were specifically upregulated in response to rAAV1-hGAA infection. In the human myoblast study, 10 different genes were identified to be up- or down-regulated in response to the specific gain in GAA activity. As a control, the FVIII gene was identified in those samples that were infected with control rAAV1-FVIII with a p-value<0.0001. The GAA gene was not identified, as a 3′ UTR truncated form of the GAA gene was used, the deleted regions of which the Affymetrix probe sets were targeted against. On the outset, two identified genes in particular stood out as potential candidates for further investigation. The differential expression of the candidate genes was confirmed by RT-PCR. The first candidate, stomatin, is a membrane protein shown to be associated with late endosomes/lysosomes. Overexpression of stomatin has been shown to inhibit GLUT-1 glucose transporter activity. The second candidate, laforin interacting protein 1, has phosphatase and carbohydrate binding sites and is associated with laforin. A lack of laforin is associated with Lafora disease, a disorder or glycogen metabolism.

5.2.4 Vector Biodistribution and Transgene Expression of Alternate Serotype Recombinant Adeno-Associated Virus Vectors

Vector is administered intravenously or via intracardiac injection to one-day-old neonate or eight-week-old Gaa^{-/-} mice. Furthermore, vector is administered directly to diaphragm in adult mice only. Intraperitoneal injection in neonate mice resulted in significant diaphragmatic transduction.

5.2.4.1 Vector Production

Packaging of rAAV serotypes 1, 2, 5, 6, 8, and 9 vectors is performed using the traditional transfection method used for AAV2 vector production described by Zolotukhin et al (1999 and 2002). Helper plasmids that retain the AAV2 rep gene and alternate AAV serotype cap genes may be used. Since helper plasmids still retain the AAV2 rep gene, all AAV plasmid constructs using AAV2 inverted terminal repeats (ITRs) are packageable with the new helper plasmids. Recombinant virus may be purified by conventional means, including for example, an iodixanol density gradient ultracentrifugation followed by anion exchange chromatography. Vector preparation purity may also be assessed by conventional means, including for example SDS-PAGE followed by silver staining to visualize protein content. Vector genome titers may be determined by conventional means, including for example, dot-blot hybridization.

5.2.4.2 Administering of Vector to Mouse Neonates

One-day-old neonate mice are anesthetized by induction of hypothermia. A 29½ G tuberculin syringe is used to deliver 1×10¹³ vector genomes/kg via the superficial temporal vein or directly into the heart (at a max. volume of 30 μt for intravenous injection or 10 μl for intracardiac injection), both of which are easily visualized during the first two days post-birth. An n of at least 5 animals are used for each serotype and route of administration (6 serotypes×2 routes×5=60 animals). Any bleeding that results from the injection may be controlled by applying light pressure to the injection site using a sterilized cotton swab until bleeding stops. Treated animals are then returned to the mothers, and normal housing and care.

5.2.4.3 Administering of Vector to Adult Mice

Eight-week-old animals are anesthetized with 2% inhaled isoflurane. As with the neonate study, an n=5 is used for each serotype/route of administration for a total of 60 animals. After reaching a surgical plane of anesthesia, an incision is made through the skin to expose the external jugular vein. Intravenous injections of 1×10¹³ vector genomes/kg are made using a 29½ G tuberculin syringe. Direct pressure to the injection site is applied using a sterile cotton swab until bleeding is stopped. The incisions are sutured closed. For direct injection into the cardiac muscle, a 22G catheter connected to a SAR-830AP rodent ventilator (CWE, Ardmore, Pa.) may be used to intubate anesthetized animals and facilitate ventilation. A left thoracotomy may be performed to expose the left ventricle. Vector is directly injected using a 29½ G tuberculin syringe and the incision is then sutured closed.

5.2.4.4 Matrix-Mediated Delivery of rAAV to Murine Diaphragm

For direct diaphragm delivery studies, an n=5 animals per serotype is used for a total of 30 mice. Individual doses (1×10¹³ vector genomes/kg) of virus is diluted in sterile phosphate buffered saline (PBS) for a total volume of 10 μL and then added to biopolymer to a final volume of 150 μL. Eight-week-old Gaa^{-/-} mice are anesthetized using 2% inhaled isoflurane and restrained supine on a warmed operating surface. After reaching a surgical plane of anesthesia, a midline incision is made, the abdominal walls retracted laterally, the gall bladder separated from the rib cage, and the liver carefully retracted from the diaphragm. While lifting the xyphoid, vector-matrix mixtures are applied directly to the abdominal surface of the diaphragm (Mah et al., 2004). The incision is sutured closed.

5.2.4.5 Tissue Analysis

Four weeks after vector administration, the diaphragm, liver, spleen, kidney, lung, heart, soleus, quadriceps, tibialis anterior, gastrocnemius and gonads are isolated and divided for the following assays: (1) lacZ enzyme detection assay, (2) detection of viral genomes, and (3) histopathological examination, as described below.

5.2.4.6 Detection of Transgene β-Galactosidase Expression

Histochemical staining with X-gal is performed as described previously (Kessler et al., 1996). Tissue is fixed in ice-cold 2% formaldehyde, 0.2% glutaraldehyde, rinsed in sterile PBS, then stained in X-gal stain solution (1 mg/mL X-gal, 5 mM potassium ferricyanide, 5 mM potassium ferrocyanide, 2 mM MgCl₂ in PBS) overnight at room temperature, protected from light. Cells expressing lacZ are stained blue.

Detection of β-galactosidase enzyme is performed on crude homogenates of tissue using the Galacto-Star™ chemiluminescent reporter gene assay system (Tropix Inc., Bedford, Mass.) per the manufacturer's instructions. Protein concentrations for tissue lysates may be determined using the Bio-Rad DC protein assay kit (Bio-Rad, Hercules, Calif.). Enzyme activities are reported as relative light units (RLU) per μg protein.

5.2.4.7 Assessment of in Vivo Biodistribution of Vector Genomes

Detection of rAAV vector genomes is assessed as described previously Mingozzi et al., 2002). Total cellular DNA is extracted from tissues using the DNeasy kit (QIAGEN, Valencia, Calif.) per the kit protocols. Co-amplification of the lacZ gene (found in the vector genome) and the endogenous murine hypoxanthine guanine phosphoribosyl transferse (Hprt) gene are performed by polymerase chain reaction (PCR) using biotinylated primers on 1.5 μg total DNA as a template. Primer pairs for lacZ (5′-CGGTGATGGTGCTGCGTT GGAG-3′ (SEQ ID NO:1) and 5′-TCGACGTTCAGACGTAGTGT-3′) (SEQ ID NO:2) and Hprt (5′-GCTGGTGAAAAGGACCTCT-3′ (SEQ ID NO:3) and 5′-CACAGGACTAGAA CACCTGC-3′ (SEQ ID NO:4)), result in final PCR products of 333 bp and 1.1 kb, respectively. In addition, standard controls include 0 (negative control), 0.01, 0.05, 0.1, 0.5 and 1 pg linearized CMV-lacZ plasmid DNA spiked into 1.5 μg control cellular DNA isolated from untreated mouse tissue. All reactions are performed under the following conditions: denaturation at 94° C. for 5 min followed by 30 cycles of denaturation at 94° C. for 1 min, annealing at 58° C. for 1 min, and extension at 72° C. for 2 min. Products are electrophoresed on a 2% agarose gel followed by transfer to a nylon membrane and visualized using the Southern-Star system (Applied Biosystems, Bedford, Mass.) as per the kit protocol. Densitometric analysis of resulting bands is performed using Scion Image Release Beta 4.0.2 software (Scion Corporation, Frederick, Md.) and ratios of lacZ/Hprt band intensity are calculated. Gene copy numbers are estimated from the standard curve generated from the standard controls.

5.2.4.8 Histopathological Examination of Tissues

After necropsy, isolated tissue sections are fixed in 10% neutral buffered formalin and processed for paraffin embedding by standard techniques. Tissue sections (5 μm thickness) are stained with hematoxylin and eosin (H&E) using standard methods.

5.3 Exemplary Therapeutic Agents Useful in Practicing the Invention

As an example of the therapeutic agents that may be delivered to mammalian muscle and cardiac tissues, the following DNA and protein sequences are included as illustrative embodiments of the present method. For the treatment of certain muscular dystrophies, expression of the human DMD gene in selected muscle tissues is preferred to ameliorate the defect and provide biologically-effective amounts of the protein to selected cells. Likewise, for Pompe's Disease, a deficiency in acid α-glucosidase (GAA), an illustrative use of the disclosed methods and composition employs a mammalian GAA gene, such as the human GAA gene identified in GenBank to express biologically- and therapeutically-effective amounts of the polypeptide in selected cells. These are but two examples of human therapeutic genes which are contemplated to find utility in the practice of the present invention.

(SEQ ID NO:5)
Human GAA Gene Sequence REF: GenBank NM_000152)
GCGCCTGCGCGGGAGGCCGCGTCACGTGACCGACCGCGGCCCCGCCCCGCGACGAGCTCCCGCCGGTCACGTGA
CCCGCCTCTGCGCGCCCCCGGGCACGACCCCGGAGTCTCCGCGGGCGGCCAGGGCGCGCGTGCGCGGAGGTGAG
CCGGGCCGGGGCTGCGGGGCTTCCCTGAGCGCGGGCCGGGTCGGTGGGGCGGTCGGCTGCCCGCGCCGGCCTCT
CAGTTGGGAAAGCTGAGGTTGTCGCCGGGGCCGCGGGTGGAGGTCGGGGATGAGGCAGCAGGTAGGACAGTGAC
CTCGGTGACGCGAAGGACCCCGGGCACCTCTAGGTTCTCCTCGTCCGCCCGTTGTTCAGCGAGGGAGGCTCTGG
GCCTGCCGCAGCTGAGGGGGAAACTGAGGCACGGAGCGGGCCTGTAGGAGCTGTCCAGGCCATCTCCAACCATG
GGAGTGAGGCACCCGCCCTGCTCCCACCGGCTCCTGGCCGTCTGCGCCCTCGTGTCGTTGGCAACCGCTGCACT
CCTGGGGCACATCCTAGTCCATGATTTCCTGCTGGTTCCCCGAGAGCTGAGTGGCTCCTCCCCAGTCCTGGAGG
AGACTCACGGAGCTCACCAGCAGGGAGCCAGGAGACCAGGGCCCCGGGATGCCCAGGCAGAGCCCGGCGGTCCC
AGAGCAGTGCCCACACAGTGCGACGTCCCCGCCAACAGCCGCTTCGATTGCGCCCCTGACAAGGCCATCACCCA
GGAACAGTGCGAGGCCCGCGGCTGCTGCTACATCCCTGCAAAGGAGGGGCTGCAGGGAGCCCAGATGGGGCAGC
CCTGGTGCTTCTTCCCACGCAGCTACCCCAGCTACAAGCTGGAGAACCTGAGCTCCTCTGAAATGGGCTACACG
GCCACCCTGACCCGTACCACCCCCACCTTCTTCCCCAAGGACATCCTGACCCTGCGGCTGGACGTGATGATGGA
GACTGAGAACCGCCTCCACTTCACGATCAAAGATCCAGCTAACAGGCGGTACGAGGTGCCCTTGGAGACCCCGC
GTGTGCACAGCCGGGCACCGTCCCCACTCTACAGCGTGGAGTTCTCCGAGGAGCCCTTCGGGGTGATCGTGCAC
CGGCAGCTGGACGGCCGCGTGCTGCTGAACACGACGGTGGCGCCCCTGTTCTTTGCGGACCAGTTCGTTCAGCT
GTCCACCTCGCTGCCCTCGCAGTATATCACAGGCCTCGCCGAGCACCTCAGTCCCCTGATGGTCAGCACCAGCT
GGACCAGGATCACCCTGTGGAACCGGGACCTTGCGCCCACGCGCGGTGCGAACCTCTACGGGTCTCACCCTTTC
TACCTGGCGCTGGAGGACGGCGGGTCGGCACACGGGGTGTTGCTGCTAAACAGCAATGCCATGGATGTGGTCCT
GCAGCCGAGCCCTGCCCTTAGCTGGAGGTCGACAGGTGGGATCCTGGATGTCTACATCTTCCTGGGCCCAGAGC
CCAAGAGGGTGGTGCAGCAGTACCTGGACGTTGTGGGATACGCGTTCATGCCGCCATACTGGGGCCTGGGCTTC
CACGTGTGCCGCTGGGGCTACTCCTCCACCGCTATCACCCGCCAGGTGGTGGAGAACATGACCAGGGCCCACTT
CCCCGTGGACGTCCAATGGAACGACCTGGACTACATGGACTCCCGGAGGGACTTCAGGTTCAACAAGGATGGCT
TCCGGGAGTTCCCGGCCATGGTGCAGGAGCTGCACCAGGGCGGCCGGCGCTACATGATGATCGTGGATCCTGCC
ATCAGCAGCTCGGGCGCTGCCGGGAGCTACAGGCCCTACGACGAGGGTCTGCGGAGGGGGGTTTTCATCACCAA
CGAGACCGGCCAGCCGCTGATTGGGAAGGTATGGCCCGGGTCCACTGCCTTCCCCGACTTCACCAACCCCACAG
CCCTGGCCTGGTGGGAGGACATGGTGGCTGAGTTCCATGACCAGGTGCCCTTCGACGGCATGTGGATTGACATG
AACGAGCCTTCCAACTTCATCAGAGGCTCTGAGGACGGCTGCCCCAACAATGAGCTGGAGAACCCACCCTACGT
GCCTGGGGTGGTTGGGGGGACCCTCCAGGCGGCCACCATCTGTGCCTCCAGCCACCAGTTTCTCTCCACACACT
ACAACCTGCACAACCTCTACGGCCTGACCGAAGCCATCGCCTCCCACAGGGCGCTGGTGAAGGCTCGGGGGACA
CGCCCATTTGTGATCTCCCGCTCGACCTTTGCTGGCCACGGCCGATACGCCGGCCACTGGACGGGGGACGTGTG
GAGCTCCTGGGAGCAGCTCGCCTCCTCCGTGCCAGAAATCCTGCAGTTTAACCTGCTGGGGGTGCCTCTGGTCG
GGGCCGACGTCTGCGGCTTCCTGGGCAACACCTCAGAGGAGCTGTGTGTGCGCTGGACCCAGCTGGGGGCCTTC
TACCCCTTCATGCGGAACCACAACAGCCTGCTCAGTCTGCCCCAGGAGCCGTACAGCTTCAGCGAGCCGGCCCA
GCAGGCCATGAGGAAGGCCCTCACCCTGCGCTACGCACTCCTCCCCCACCTCTACACACTGTTCCACCAGGCCC
ACGTCGCGGGGGAGACCGTGGCCCGGCCCCTCTTCCTGGAGTTCCCCAAGGACTCTAGCACCTGGACTGTGGAC
CACCAGCTCCTGTGGGGGGAGGCCCTGCTCATCACCCCAGTGCTCCAGGCCGGGAAGGCCGAAGTGACTGGCTA
CTTCCCCTTGGGCACATGGTACGACCTGCAGACGGTGCCAATAGAGGCCCTTGGCAGCCTCCCACCCCCACCTG
CAGCTCCCCGTGAGCCAGCCATCCACAGCGAGGGGCAGTGGGTGACGCTGCCGGCCCCCCTGGACACCATCAAC
GTCCACCTCCGGGCTGGGTACATCATCCCCCTGCAGGGCCCTGGCCTCACAACCACAGAGTCCCGCCAGGAGCC
CATGGCCCTGGCTGTGGCCCTGACCAAGGGTGGAGAGGCCCGAGGGGAGCTGTTCTGGGACGATGGAGAGAGCC
TGGAAGTGCTGGAGCGAGGGGCCTACACACAGGTCATCTTCCTGGCCAGGAATAACACGATCGTGAATGAGCTG
GTACGTGTGACCAGTGAGGGAGCTGGCCTGCAGCTGCAGAAGGTGACTGTCCTGGGCGTGGCCACGGCGCCCCA
GCAGGTCCTCTCCAACGGTGTCCCTGTCTCCAACTTCACCTACAGCCCCGACACCAAGGTCCTGGACATCTGTG
TCTCGCTGTTGATGGGAGAGGAGTTTCTCGTCAGCTGGTGTTAGCCGGGCGGAGTGTGTTAGTCTCTCCAGAGG
GAGGCTGGTTCCCCAGGGAAGCAGAGCCTGTGTGCGGGCAGCAGCTGTGTGCGGGCCTGGGGGTTGGATGTGTC
ACCTGGAGCTGGGCACTAACCATTCCAAGCCGCCGCATCGCTTGTTTCCACCTCCTGGGCCGGGGCTCTGGCCC
CCAACGTGTCTAGGAGAGCTTTCTCCCTAGATCGCACTGTGGGCCGGGGCCTGGAGGGCTGCTCTGTGTTAATA
AGATTGTAAGGTTTGCCCTCCTCACCTGTTGCCGGCATGCGGGTAGTATTAGCCACCCCCCTCCATCTGTTCCC
AGCACCGGAGAAGGGGGTGGTCAGGTGGAGGTGTGGGGTATGCACCTGAGCTCCTGGTTCGCGCCTGCTGCTCT
GCCCCAACGCGACCGCTTCGCGGCTGCCCAGAGGGCTGGATGCCTGCCGGTCCCCGAGCAAGCCTGGGAAGTCA
GGAAAATTCAGAGGACTTGGGAGATTCTAAATCTTAAGTGCAATTATTTTAATAAAAGGGGCATTTGGAATC

53.2 Human GAA Protein

(SEQ ID NO:6)
Human GAA Protein-REF: GenBank NM_000152
MGVRHPPCSHRLLAVCALVSLATIALLGHILLHDFLLVPRELSGSSPVLEETHPAHQQGASRPGPRDAQAHPGR
PRAVPTQCDVPPNSRFDCAPDKAITQEQCEARGCCYIPAKQGLQGAQMGQPWCFFPPSYPSYKLENLSSSEMGY
TATLTRTTPTFFPKDILTLRLDVMMETENRLHFTIKDPANRRYEVPLETPRVHSRAPSPLYSVEFSEEPFGVIV
HRQLDGRVLLNTTVAPLFFADQFLQLSTSLPSQYITGLAEHLSPLMLSTSWTRITLWNRDLAPTPGANLYGSHP
FYLALEDGGSAHGVFLLNSNANDVVLQPSPALSWRSTGGTLDVYIFLGPEPKSVVQQYLDVVGYPFMPPYWGLG
FHLCRWGYSSTATTRQVVENMTRAHFPLDVQWNDLDYMDSRRDFTFNKDGFRDFPAMVQELHQGGRRYMMTVDP
ATSSSGPAGSYRPYDEGLRRGVFITNETGQPLTGKVWPGSTAFPDFTNPTALAWWEDMVAEFHDQVPFDGMWID
MNEPSNFIRGSEDGGPNNELENPPYVPGVVGGTLQAATTCASSHQFLSTHYNLHNLYGLTEAIASHRALVKARG
TRPFVISRSTFAGHGRYAGHWTGDVWSSWEQLASSVPEILQFNLLGVPLVGADVCGFLGNTSEELCVRWTQLGA
FYPFMRNHNSLLSLPQEPYSFSEPAQQAMRKALTLRYALLPHLYTLFHQAHVAGETVARPLFLEFPKDSSTWTV
DHQLLWGEALLITPVLQAGKAEVTGYFPLGTWYDLQTVPIEALGSLPPPPAAPREPATHSEGQWVTLPAPLDTI
NVHLRAGYIIPLQGPGLTTTESRQQPMALAVALTKGGEARGELFWDDGESLEVLERGAYTQVIFLARNNTIVNE
LVRVTSEGAGLQLQKVTVLGVATAPQQVLSNGVPVSNFTYSPDTKVLDICVSLLMGEQFLVSWC

5.3.3 Human DMD Dystrophin Gene (Duchenne Becker Type)

(SEQ ID NO:7)
Human DMD (Dystrophin) Gene Sequence REF: GenBank M18533
TTTTCAAAATGCTTTGGTGGGAAGAAGTAGAGGACTGTTATGAAAGAGAAGATGTTCAAAAGAAAACATTCACA
AAATGGGTAAATGCACAATTTTCTAAGTTTGGGAAGCAGCATATTGAGAACCTCTTCAGTGACCTACAGGATGG
GAGGCGGCTCCTAGAGCTCCTCGAAGGCCTGACAGGGCAAAAACTGCCAAAAGAAAAAGGATCCACAAGAGTTC
ATGCCCTGAACAATGTCAACAAGGCACTGCGGGTTTTGCAGAACAATAATGTTGATTTAGTGAATATTGGAAGT
ACTGACATCGTAGATGGAAATCATAAACTGACTCTTGGTTTGATTTGGAATATAATCCTCCACTGGCAGGTCAA
AAATGTAATGAAAAATATCATGGCTGGATTGGAACAAACCAACAGTGAAAAGATTCTGCTGAGCTGGGTCGGAC
AATGAACTCGTAATTATCCACAGGTTAATGTAATCAAGTTCACCACCAGCTGGTCTGATGGCCTGGCTTTGAAT
GCTCTCATCCATAGTGATAGGCCAGACCTATTTGACTGGAATAGTGTGGTTTGCCAGCAGTCAGCCACACAACG
ACTGGAACATGCATTCAACATCGCCAGATATCAATTAGGCATAGAGAAACTACTCGATCCTGAAGATGTTGATA
CCACCTATCGAGATAAGAAGTCCATCTTAATGTACATCACATCACTCTTCCAAGTTTTGCCTCAAGAAGTGAGC
ATTGAAGCCATCCAGGAAGTGGAAATGTTGCCAAGGCCACCTAAAGTGACTAAAGAAGAACATTTTCAGTTACA
TCATCAAATGCACTATTCTCAACAGATCACGGTCAGTCTAGCACAGGGATATGAGAGAACTTCTTCCCCTAAGC
CTCGATTCAAGAGCTATGCCTACACACAGGCTGCTTATGTCACCACCTCTGACCCTACACGGAGCCCATTTCCT
TCACAGCATTTGGAAGCTCCTGAAGACAAGTCATTTGGCAGTTCATTGATGGAGAGTGAAGTAAACCTGGACCG
TTATCAAACAGCTTTAGAAGAAGTATTATCGTGGCTTCTTTCTGCTGAGGACACATTGCAAGCACAAGGAGAGA
TTTCTAATGATGTGGAAGTGGTGAAAGACCAGTTTCATACTCATGAGGGGTACATGATGGATTTGACAGCCCAT
CAGGGCCGGGTTGGTAATATTCTACAATTGGGAAGTAAGCTGATTGGAACAGGAAAATTATCAGAAGATGAAGA
AACTGAAGTACAAGAGCAGATGAATCTCCTAAATTCAAGATGGGAATGCCTCAGGGTAGCTAGGATGGAAAAAC
AAAGCAATTTACATAGAGTTTTAATGGATCTCCAGAATCAGAAACTGAAAGAGTTGAATGACTGGCTAACAAAA
ACAGAAGAAAGAACAAGGAAAATGGAGGAAGAGCCTCTTGGACCTGATCTTGAAGACCTAAAACGCCAAGTACA
ACAACATAAGGTGCTTCAAGAAGATCTAGAACAAGAACAAGTCAGGGTCAATTCTCTCACTCACATGGTGGTGG
TAGTTGATGAATCTAGTGGAGATCACGCAACTGCTGCTTTGGAAGAACAACTTAAGGTATTGGGAGATCGATGG
GCAAACATCTGTAGATGGACAGAAGACCGCTGGGTTCTTTTACAAGACATCCTTCTCAAATGGCAACGTCTTAG
TGAAGAACAGTGCCTTTTTAGTGCATGGCTTTCAGAAAAAGAAGATGCAGTGAACAAGATTCACACAACTGGCT
TTAAAGATCAAAATGAAATGTTATCAAGTCTTCAAAAACTGGCCGTTTTAAAAGCGGATCTAGAAAAGAAAAAG
CAATCCATGGGCAAACTGTATTCACTCAAACAAGATCTTCTTTCAACACTGAAGAATAAGTCAGTGACCCAGAA
GACGGAAGCATGGCTGGATAACTTTGCCCGGTGTTGGGATAATTTAGTCCAAAAACTTGAAAAGAGTACAGCAC
AGATTTCACAGGCTGTCACCACCACTCAGCCATCACTAACACAGACAACTGTAATGGAAACAGTAACTACGGTG
ACGACAAGGGAACAGATCCTGGTAAAGCATGCTCAAGAGGAACTTCCACCACCACCTCCCCAAAAGAAGAGGCA
GATTACTGTGGATTCTGAAATTAGGAAAAGGTTGGATGTTGATATAACTGAACTTCACAGCTGGATTACTGGCT
CAGAAGCTGTGTTGCAGAGTGCTGAATTTGCAATCTTTCGGAAGGAAGGCAACTTCTCAGACTTAAAAGAAAAA
GTCAATGGCATAGAGCGAGAAAAAGCTGAGAAGTTCAGAAAACTGCAAGATGCCAGCAGATCAGCTCAGGCCCT
GGTGGAACAGATGGTGAATGAGGGTGTTAATGCAGATAGCATCAAACAAGCCTCAGAACAACTGAACAGCCGGT
GGATCGAATTCTGCCAGTTGCTAAGTGAGAGACTTAACTGGCTGGAGTATCAGAACAACATCATCGCTTTCTAT
AATCAGCTACAACAATTGGAGCAGATGACAACTACTGCTGAAAACTGGTTGAAAATCCAACCCAGCAGCCCATC
AGAGCCAACAGCAATTAAAAGTCAGTTAAAAATTTGTAAGGATGAAGTCAAGCGGCTATCAGGTCTTCAACCTC
AAATTGAACGATTAAAAATTCAAAGCATAGCCCTGAAAGAGAAAGGACAAGGACCCATGTTCCTGGATGCAGAG
TTTGTGGCCTTTACAAATCATTTTAAGCAAGTCTTTTCTGATGTGCAGGCCAGAGAGAAAGAGCTACAGACAAT
TTTTGACACTTTGCCACCAATGCGCTATCAGGAGACCATGAGTGCCATCAGGACATGGGTCCAGCAGTCAGAAA
CCAAACTCTCCATACCTCAACTTAGTGTCACCGACTATGAAATCATGGAGCAGAGACTCGGGGAATTGCAGGCT
TTACAAAGTTCTCTGCAAGAGCAACAAAGTGGCCTATACTATCTCAGGACCACTGTGAAAGAGATGTCGAAGAA
AGCGCCCTCTGAAATTAGCCGGAAATATCAATGAGAATTTGAAGAAATTGAGGGACGCTGGAAGAAGGTCTCCT
GCCAGCTGGTTGAGCATTGTCAAAAGCTAGAGGAGCAAATGAATAAACTCCGAAAATTTCAGAATCACATACAA
ACGCTGAAGAAATGGATGGCTGAAGTTGATGTTTTTCTGAAGGAGGAATGGCCTGGCCTTGGGGATTCAGAAAT
TCTAAAAAAGCAGCTGAAACAGTGCAGAGTTTTAGTCAGTGATATTCAGACAATTCAGCCCAGTCTAAACAGTG
TCAATGAAGGTGGGCAGAAGATAAAGAATGAAGCAGAGCCAGAGTTTGGTTCGAGACTTGAGACAGAACTCAAA
GAACTTAACACTCAGTGGGATCACATGTGCCAACAGGTCTATGGCAGAAAGGAGGCCTTGAAGGGAGGTTTGGA
GAAAACTGTAAGCCTCCAGAAAGATCTATCAGAGATGCACGAATGGATGACACAAGCTGAAGAAGAGTATCTTG
AGAGAGATTTTGAATATAAAACTCCAGATGAATTACAGAAAGCAGTTGAAGAGATGAAGAGAGCTAAAGAAGAG
GCCCAACAAAAAGAAGGGAAAGTGAAACTCGTTACTGAGTGTGTAAATAGTGTCATAGCTCAAGGTCGACCTGT
AGCACAAGAGGCCTTAAAAAAGGAACTTGAAACTCTAACCACCAACTACGAGTGGCTCTGCACTAGGCTGAATG
GGAAATGCAAGACTTTGGAAGAAGTTTGGGCATGTTGGCATGAGTTATTGTCATACTTGGAGAAAGCAAACAAG
TGGCTAAATGAAGTAGAATTTAAACTTAAAACCACTGAAAACATTCCTGGCGGAGCTGAGGAAATCTCTGAGGT
GCTAGATTCACTTGAAAATTTGATGCGACATTCAGAGGATAACCCAAATCAGATTCGCATATTGGCACAGACCC
TAACAGATGGCGGAGTCATGGATGAGCTAATCAATGAGGAACTTGAGACATTTAATTCTCGTTGGAGGGAAGTA
CATGAAGAGGCTGTAAGGAGGCAAAAGTTGCTTGAACAGAGCATCCAGTCTGCCCAGGAGACTGAAAAATCGTT
ACACTTAATCCAGGAGTCCCTGACATTCATTGACAAGCAGTTGGCAGCTTATATTGCAGACAAGGTGGACGCAG
CTCAAATGCCTCAGGAAGCCCAGAAAATGCAATCTGATTTGACAAGTCATGAGATCAGTTTAGAAGAAATGAAG
AAACATAATCAGGGGAAGGAGGCTGCCCAAAGAGTCCTGTCTCAGATTGATGTTGCACAGAAAAAATTACAAGA
TGTCTCCATGAAGTTTCGATTATTCCAGAAACCAGCCAATTTTGAGCTGCGTCTACAAGAAAGTAAGATGATTT
TAGATGAAGTGAAGATGCACTTGCCTGCATTGGAAACAAAGAGTGTGGAAGAGGAAGTAGTACAGTCACAGCTA
AATCATTGTGTGAACTTGTATAAAAGTCTGAGTGAAGTGAAGTCTGAAGTGGAAATGGTGATAAAGACTGGACG
TCAGATTGTAGAGAAAAAGCAGACGGAAAATCCCAAAGAACTTGATGAAAGAGTAACAGCTTTGAAATTGCATT
ATAATGAGCTGGGAGCAAAGGTAACAGAAAGAAAGCAACAGTTGGAGAAATGCTTGAAATTGTCCCGTAAGATG
CGAAAGGAAATGAATGTCTTGACAGAATGGCTGGCAGCTACAGATATGGAATTGACAAAGAGATCAGCAGTTGA
AGGAATGCCTAGTAATTTGGATTCTGAAGTTGCCTGGGGAAAGGCTACTCAAAAAGAGATTGAGAAACAGAAGG
TGCACCTGAAGAGTATCACAGAGGTAGGAGAGGCGTTGAAAACAGTTTTGGGCAAGAAGGAGACGTTGGTGGAA
GATAAACTCAGTCTTCTGAATAGTAACTGGATAGCTGTCACCTCCCGAGCAGAAGAGTGGTTAAATCTTTTGTT
GGAATACCAGAAACACATGGAAACTTTTGACCAGAATGTGGACCAGATCACAAAGTGGATCATTCAGGCTGACA
GACTTTTGGATGAATCAGAGAAAAAGAAACCCCAGCAAAAAGAAGACGTGCTTAAGCGTTTAAAGGCAGAACTG
AATGACATACGCCCAAAGGTGGACTCTACACGTGACCAAGCAGCAAACTTGATGGCAAACCGCGGTGACCACTG
CAGGAAATTAGTAGAGCCCCAAATCTCAGAGCTCAACCATCGATTTGCAGCCATTTCACACAGAATTAAGACTG
GAAAGGCCTCCATTCCTTTGAAGGAATTGGAGCAGTTTAACTCAGATATACAAAAATTGCTTGAACCACTGGAG
GCTGAAATTCAGCAGGGGGTGAATCTGAAAGAGGAAGACTTCAATAAAGATATGAATGAAGACAATGAGGGTAC
TGTAAAAGAATTGTTGCAAAGAGGAGACAACTTACAACAAAGAATCACAGATGAGAGAAAGAGAGAGGAAATAA
AGATAAAACAGCAGCTGTTACAGACAAAACATAATGCTCTCAAGGATTTGAGGTCTCAAAGAAGAAAAAAGGGT
CTAGAAATTTCTGATCAGTGGTATCAGTACAAGAGGCAGGCTGATGATCTCCTGAAATGCTTGGATGACATTGA
AAAAAAATTAGCCAGCCTACCTGAGCCCAGAGATGAAAGGAAAATAAAGGAAATTGATCGGGAATTGCAGAAGA
AGAAAGAGGAGCTGAATGCAGTGCGTAGGCAAGCTGAGGGGTTGTCTGAGGATGGGGCCGCAATGGCAGTGGAG
CCAACTCAGATCCAGCTCAGCAAGCGCTGGCGGGAAATTGAGAGCAAATTTGCTCAGTTTCGAAGACTCAACTT
TGCACAAATTCACACTGTCCGTGAAGAAACGATGATGGTGATGACTGAAGACATGCCTTTGGAAATTTCTTATG
TGCCTTCTACTTATTTGACTGAAATCACTCATGTCTCACAAGCCCTATTAGAAGTGGAACAACTTCTCAATGCT
GCTGACCTCTGTGCTAAGGACTTTGAAGATCTCTTTAAGCAAGAGGAGTCTCTGAAGAATATAAAAGATAGTCT
ACAACAAAGCTCAGGTCGGATTGACATTATTCATAGCAAGAAGACAGCAGCATTGCAAAGTGCAACGCCTGTGG
AAAGGGTGAAGCTACAGGAAGCTCTCTCCCAGCTTGATTTCCAATGGGAAAAAGTTAACAAAATGTAGAAGGAC
CGACAAGGGGGATTTGAGAGATCTGTTGAGAAATGGCGGCGTTTTCATTATGATATAAAGATATTTAATCAGTG
GCTAACAGAAGCTGAACAGTTTCTCAGAAAGACACAAATTCCTGAGAATTGGGAACATGCTAAATACAAATGGT
ATCTTAAGGAAGTCCAGGATGGCATTGGGCAGCGGCAAACTGTTGTGAGAACATTGAATGCAACTGGGGAAGAA
ATAATTCAGGAATCCTGAAAAACAGATGCCAGTATTCTACAGGAAAAATTGGGAAGCGTGAATCTGCGGTGGCA
GGAGGTCTGGAAACAGCTGTGAGACAGAAAAAAGAGGCTAGAAGAACAAAAGAATATCTTGTCAGAATTTCAAA
GAGATTTAAATGAATTTGTTTTATGGTTGGAGGAAGCAGATAACATTGCTAGTATCCCACTTGAACCTGGAAAA
GAGCAGCAAGTAAAAGAAAAGCTTGAGCAAGTCAAGTTACTGGTGGAAGAGTTGCCCCTGCGCCAGGGAATTCT
CAAACAATTAAATGAAACTGGAGGACCCGTGCTTGTAAGTGCTCCCATAAGCCCAGAAGAGCAAGATAAAGTTG
AAAATAAGCTCAAGCAGACAAATCTCCAGTGGATAAAGGTTTCCAGAGCTTTACCTGAGAAACAAGGAGAAATT
GAAGCTCAAATAAAAGACCTTGGGCAGGTTGAAAAAAAGCTTGAAGACCTTGAAGAGCAGTTAAATGATCTGCT
GCTGTGGTTATCTCCTATTAGGAATCAGTTGGAAATTTATAACCAACCAAACCAAGAAGGACCATTTGACGTTC
AGGAAACTGAAATAGCAGTTCAAGCTAAACAACCGGATGTGGAAGAGATTTTGTCTAAAGGGCAGCATTTGTAC
AAGGAAAAACCAGCCACTCAGCCAGTGAAGAGGAAGTTAGAAGATCTGAGCTCTGAGTGGAAGGCGGTAAACCG
TTTACTTCAAGAGCTGAGGGCAAAGCAGCCTGACCTAGCTCCTGGACTGACCACTATTGGAGCCTCTCCTACTC
AGACTGTTACTCTGGTGACACAACCTGTGGTTACTAAGGAAACTGCCATCTCCAAACTAGAAATGCCATCTTCC
TTGATGTTGGAGGTACCTGCTCTGGCAGATTTGAACCGGGCTTGGACAGAACTTACCGACTGGCTTTCTCTGCT
TGATCAAGTTATAAAATCACAGAGGGTGATGGTGGGTGACCTTGAGGATATCAACGAGATGATCATCAAGCAGA
AGGCAACAATGCAGGATTTGGAACAGAGGCGTCCCCAGTTGGAAGAACTCATTACCGCTGCCCAAAATTTGAAA
AACAAGACCAGCAATCAAGAGGCTAGAACAATCATTACGGATCGAATTGAAAGAATTCAGAATCAGTGGGATGA
AGTACAAGAACACCTTCAGAACCGGAGGCAACAGTTGAATGAAATGTTAAAGGATTCAACACAATGGCTGGAAG
CTAAGGAAGAAGCTGAGCAGGTCTTAGGACAGGCCAGAGCCAAGCTTGAGTCATGGAAGGAGGGTCCCTATACA
GTAGATGCAATCCAAAAGAAAATCACAGAAACCAAGCAGTTGGCCAAAGACCTCCGCCAGTGGCAGACAAATGT
AGATGTGGCAAATGACTTGGCCCTGAAACTTCTCCGGGATTATTCTGCAGATGATACCAGAAAAGTCCACATGA
TAACAGAGAATATCAATGCCTCTTGGAGAAGCATTCATAAAAGGGTGAGTGAGCGAGAGGCTGCTTTGGAAGAA
ACTGATAGATTACTGCAACAGTTCCCCCTGGACCTGGAAAAGTTTCTTGCCTGGCTTACAGAAGCTGAAAGAAC
TGCGAATGTCCTACAGGATGCTACCCGTAAGGAAAGGCTCCTAGAAGACTCCAAGGGAGTAAAAGAGGTGATGA
AACAATGGCAAGACCTCCAAGGTGAAATTGAAGCTCACACAGATGTTTATCACAACCTGGATGAAAACAGCCAA
AAAATCCTGAGATCCCTGGAAGGTTCCGATGATGCAGTCCTGTTACAAAGACGTTTGGATAACATGAACTTCAA
GTGGAGTGAACTTCGGAAAAAGTCTCTCAACATTAGGTCCCATTTGGAAGCCAGTTCTGACCAGTGGAAGCGTC
TGCACCTTTCTCTGCAGGAACTTCTGGTGTGGCTACAGCTGAAAGATGATGAATTAAGCCGGCAGGGACCTATT
GGAGGCGACTTTCCAGCAGTTCAGAAGCAGAACGATGTACATAGGGCCTTCAAGAGGGAATTGAAAACTAAAGA
ACCTGTAATCATGAGTACTCTTGAGACTGTACGAATATTTCTGACAGAGCAGCGTTTGGAAGGACTAGAGAAAC
TCTACCAGGAGCCCAGAGAGCTGCCTCCTGAGGAGAGAGCCCAGAATGTCACTCGGCTTCTACGAAAGCAGGCT
GAGGAGGTCAATACTGAGTGGGAAAAATTGAACCTGCACTCCGCTGACTGGCAGAGAAAAATAGATGAGACCCT
TGAAAGACTCCAGGAACTTCAAGAGGCCACGGATGAGCTGGACCTCAAGCTGCGCCAAGCTGAGGTGATCAAGG
GATCCTGGCAGCCCGTGGGCGATCTCCTCATTGACTCTCTCCAAGATCACCTCGAGAAAGTCAAGGCACTTCGA
GGAGAAATTGCGCCTCTGAAAGAGAACGTGAGCGACGTCAATGACCTTGCTCGCCAGCTTACCACTTTGGGCAT
TCAGCTCTCACCGTATAACCTCAGCACTCTGGAAGACCTGAACACCAGATGGAAGCTTCTGCAGGTGGCCGTCG
AGGACCGAGTCAGGCAGCTGCATGAAGCCCACAGGGACTTTGGTCCAGCATGTCAGCACTTTCTTTCCACGTCT
GTCCAGGGTCCCTGGGAGAGAGCCATCTCGCCAAACAAAGTGGCCTACTATATCAACCACGAGACTCAAACAAC
TTGCTGGGAGCATCCCAAAATGAGAGAGCTCTACCAGTCTTTAGCTGACCTGAATAATGTCAGATTCTCAGCTT
ATAGGACTGCCATGAAACTCCGAAGACTGGAGAAGGCCCTTTGCTTGGATCTCTTGAGCCTGTCAGCTGCATGT
GATGCCTTGGACCAGCACAACCTCAAGCAAAATGACCAGCCCATGGATATCCTGCAGATTATTAATTGTTTGAC
CACTATTTATGACCGCCTGGAGCAAGAGCACAACAATTTGGTCAACGTCCCTCTCTGCGTGGATATGTGTCTGA
ACTGGCTGCTGAATGTTTATGATACGGGACGAACAGGGAGGATCCGTGTCCTGTCTTTTAAAACTGGCATCATT
TCCCTGTGTAAAGCACATTTGGAAGACAAGTACAGATACCTTTTCAAGCAAGTGGCAAGTTCAACAGGATTTTG
TGACCAGCGCAGGCTGGGCCTCCTTCTGCATGATTCTATCCAAATTCCAAGACAGTTGGGTGAAGTTGCATCCT
TTGGGGGCAGTAACATTGAGCCAAGTGTCCGGAGCTGCTTCCAATTTGCTAATAATAAGCCAGAGATCGAAGCG
GCCCTCTTCCTAGACTGGATGAGACTGGAACCCCAGTCCATGGTGTGGCTGCCCGTCCTGCAGAGAGTGGCTGC
TGCAGAAACTGCCAAGCATCAGGCCAAATGTAACATCTGCAAAGAGTGTCCAATCATTGGATTCAGGTACAGGA
GTCTAAAGCACTTTAATTATGACATCTGCCAAAGCTGCTTTTTTTCTGGTCGAGTTGCAAAAGGCCATAAAATG
CACTATCCCATGGTGGAATATTGCACTCCGACTACATCAGGAGAAGATGTTCGAGACTTTGCCAAGGTACTAAA
AAACAAATTTCGAACCAAAAGGTATTTTGCGAAGCATCCCCGAATGGGCTACCTGCCAGTGCAGACTGTCTTAG
AGGGGGACAACATGGAAACTCCCGTTACTCTGATCAACTTCTGGCCAGTAGATTCTGCGCCTGCCTCGTCCCCT
CAGCTTTCACACGATGATACTCATTCAGGCATTGAACATTATGCTAGCAGGCTAGCAGAAATGGAAAACAGCAA
TGGATCTTATCTAAATGATAGCATCTCTCCTAATGAGAGCATAGATGATGAACATTTGTTAATCCAGCATTACT
GCCAAAGTTTGAACCAGGACTCCCCCCTGAGCCAGCCTCGTAGTCCTGCCCAGATCTTGATTTCCTTAGAGAGT
GAGGAAAGAGGGGAGCTAGAGAGAATCCTAGCAGATCTTGAGGAAGAAAACAGGAATCTGCAAGCAGAATATGA
CCGTCTAAAGCAGCAGCACGAACATAAAGGCCTGTGCCCACTGCCGTCCCCTCCTGAAATGATGCCCACCTCTC
CCGAGAGTCCCCGGGATGCTGAGCTCATTGCTGAGGCCAAGCTACTGGGTCAACACAAAGGCCGCCTGGAAGCC
AGGATGCAAATCCTGGAAGACCACAATAAACAGCTGGAGTCACAGTTACACAGGCTAAGGCAGCTGGTGGAGCA
ACCCCAGGGAGAGGCCAAAGTGAATGGCAGAAGGGTGTCCTCTCCTTCTAGCTCTCTAGAGAGGTCCGACAGCA
GTCAGCCTATGCTGCTCCGAGTGCTTGGCAGTCAAAGTTGGGACTCGATGGGTGAGGAAGATCTTCTCAGTCCT
GGCCAGGAGACAAGCACAGGGTTAGAGGAGGTGATGGAGCAACTCAACAAGTCCTTCCCTAGTTCAAGAGGAAG
AAATAGCCCTGGAAAGGCAATGACAGAGGACACAATGTAG

53.4 Human DMD Dystrophin Polypeptide (Duchenne Becker Type)

(SEQ ID NO:8)
Human DMD (Dystrophin) Protein Sequence REF: GenBank M18533
MLWWEEVEDCYEREDVQKKTFTKWVNAQFSKFGKQHIENLFSDLQDGRRLLDLLEGLTGQKLPKEKGSTRVHAL
NNVNKALRVLQNNNVDLVNIGSTDIVDGNHKLTLGLIWNIILHWQVKNVMKNIMAGLQQTNSEKILLSWVRQST
RNYPQVNVINFTTSWSDGLALNALIHSHRPDLFDWNSVVCQQSATQRLEHAFNIARYQLGIEKLLDPEDVDTTY
PDKKSILMYITSLFQVLPQQVSIEAIQEVEMLPRPPKVTKEEHFQLHHQMHYSQQITVSLAQGYERTSSPKPRF
KSYAYTQAAYVTTSDPTRSPFPSQHLEAPEDKSFGSSLMESEVNLDRYQTALEEVLSWLLSAEDTLQAQGEISN
DVEVVKDQFHTHEGYMMDLTAHQGRVGNILQLGSKLIGTGKLSEDEETEVQEQMNLLNSRWECLRVASMEKQSN
LHRVLMDLQNQKLKELNDWLTKTEERTRKMEEEPLGPDLEDLKRQVQQHKVLQEDLEQEQVRVNSLTHMVVVVD
ESSGDHATAALEEQLKVLGDRWANICRWTEDRWVLLQDILLKWQRLTEEQCLFSAWLSEKEDAVNKIHTTGFKD
QNEMLSSLQKLAVLKADLEKKKQSMGKLYSLKQDLLSTLKNKSVTQKTEAWLDNFARCWDNLVQKLEKSTAQIS
QAVTTTQPSLTQTTVMETVTTVTTREQILVKHAQEELPPPPPQKKRQITVDSEIRKRLDVDITELHSWITRSEA
VLQSPEFAIFRKEGNFSDLKEKVNAIEREKAEKFRKLQDASRSAQALVEQMVNEGVNADSIKQASEQLNSRWIE
FCQLLSERLNWLEYQNNIIAFYNQLQQLEQMTTTAENWLKIQPTTPSEPTAIKSQLKICKDEVNRLSGLQPQIE
RLKIQSIALKEKGQGPMFLDADFVAFTNHFKQVFSDVQAREKELQTIFDTLPPMRYQETMSAIRTWVQQSETKL
SIPQLSVTDYEIMEQRLGELQALQSSLQEQQSGLYYLSTTVKEMSKKAPSEISRKYQSEFEEIEGRWKKLSSQL
VEHCQKLEEQMNKLRKIQNHIQTLKKWMAEVDVFLKEEWPALGDSEILKKQLKQCRLLVSDIQTIQPSLNSVNE
GGQKIKNEAEPEFASRLETELKELNTQWDHMCQQVYARKEALKGGLEKTVSLQKDLSEMHEWMTQAEEEYLERD
FEYKTPDELQKAVEEMKRAKEEAQQKEAKVKLLTESVNSVIAQAPPVAQEALKKELETLTTNYQWLCTRLNGKC
KTLEEVWACWHELLSYLEKANKWLNEVEFKLKTTENIPGGAEEISEVLDSLENLMRHSEDNPNQIRILAQTLTD
GGVMDELINEELETFNSRWRELHEEAVRRQKLLEQSIQSAQETEKSLHLIQESLTFIDKQLAAYIADKVDAAQM
PQEAQKIQSDLTSHEISLEEMKKHNQGKEAAQRVLSQIDVAQKKLQDVSMKFRLFQKPANFELRLQESKMILDE
VKMHLPALETKSVEQEVVQSQLNHCVNLYKSLSEVKSEVEMVIKTGRQIVQKKQTENPKELDERVTALKLHYNE
LGAKVTERKQQLEKCLKLSRKMRKEMNVLTEWLAATDMELTKRSAVEGMPSNLDSEVAWGKATQKEIEKQKVHL
KSITEVGEALKTVLGKKETLVEDKLSLLNSNWIAVTSRAEEWLNLLLEYQKHMETFDQNVDHITKWIIQADTLL
DESEKKKPQQKEDVLKRLKAELNDIRPKVDSTRDQAANLMANRGDHCRKLVEPQISELNHRFAAISHRIKTGKA
SIPLKELEQFNSDIQKLLEPLEAEIQQGVNLKEEDFNKDMNEDNEGTVKELLQRGDNLQQRITDERKREEIKIK
QQLLQTKHNALKDLRSQRRKKALEISHQWYQYKRQADDLLKCLDDIEKKLASLPEPRDERKIKEIDRELQKKKE
ELNAVRRQAEGLSEDGAAMAVEPTQTQLSKRWREIESKFAQFRRLNFAQIHTVREETMMVMTEDMPLEISYVPS
TYLTEITHVSQALLEVEQLLNAPDLCAKDFEDLFKQEESLKNIKDSLQQSSGRIDIIHSKKTAALQSATPVERV
KLQEALSQLDFQWEKVNKMYKDRQGRFDRSVEKWRRFHYDIKIFNQWLTEAEQFLRKTQIPENWEHAKYKWYLK
ELQDGICQRQTVVRTLNATGEEIIQQSSKTDASILQEKLGSLNLRWQEVCKQLSDRKKRLEEQKNILSEFQRDL
NEFVLWLEEADNIASIPLEPGKEQQLKEKLEQVKLLVEELPLRQGILKQLNETGGPVLVSAPISPEEQDKLENK
LKQTNLQWIKVSRALPEKQGEIEAQIKDLGQLEKKLEDLEEQLNHLLLWLSPIRNQLEIYNQPNQEGPFDVQET
EIAVQAKQPDVEEILSKGQHLYKEKPATQPVKRKLEDLSSEWKAVNRLLQELRAKQPDLAPGLTTIGASPTQTV
TLVTQPVVTKETAISKLEMPSSLMLEVPALADFNRAWTELTDWLSLLDQVIKSQRVMVGDLEDINEMTIKQKAT
MQDLEQRRPQLEELITAAQNLKNKTSNQEARTIITDRIERIQNQWDEVQEHLQNRRQQLNEMLKDSTQWLEAKE
EAEQVLGQARAKLESWKEGPYTVDAIQKKITETKQLAKDLRQWQTNVDVANDLALKLLRDYSADDTRKVHMITE
NINASWRSIHKRVSEREAALEETHRLLQQFPLDLEKFLAWLTEAETTANVLQDATRKERLLEDSKGVKELMKQW
QDLQGEIEAHTDVYHNLDENSQKILRSLEGSDDAVLLQRRLDNMNFKWSELRKKSLNIRSHLEASSDQWKRLHL
SLQELLVWLQLKDDELSRQAPIGGDFPAVQKQNDVHRAFKRELKTKEPVIMSTLETVRIFLTEQPLEGLEKLYQ
EPRELPPEERAQNVTRLLRKQAEEVNTEWEKLNLHSADWQRKIDETLERLQELQEATDELDLKLRQAEVTKGSW
QPVGDLLIDSLQDHLEKVKALRGEIAPLKENVSHVNDLARQLTTLGIQLSPYNLSTLEDLNTRWKLLQVAVEDR
VRQLHEAHRDFGPASQHFLSTSVQGPWERAISPNKVPYYINHETQTTCWDHPKMTELYQSLADLNNVRFSAYRT
AMKLRRLQKALCLDLLSLSAACDALDQHNLKQNDQPMDILQIINCLTTIYDRLEQEHNNLVNVPLCVDMCLNWL
LNVYDTGRTGRIRVLSFKTGIISLCKAHLEDKYRYLFKQVASSTGFCDQRRLGLLLHDSIQIPRQLGEVASFGG
SNIEPSVRSCFQFANNKPEIEAALFLDWMRLEPQSMVWLPVLHRVAAAETAKHQAKCNICKECPIIGFRYRSLK
HFNYDICQSCFFSGRVAKGHKMHYPMVEYCTPTTSGEDVRDFAKVLKNKFRTKRYFAKHPRMGYLPVQTVLEGD
NMETPVTLINFWPVDSAPASSPQLSHDDTHSRIEHYASRLAEMENSNGSYLNDSISPNESIDDEHLLIQHYCQS
LNQDSPLSQPRSPAQILISLESEERGELERTLADLEEENRNLQAEYDRLKQQHEHKGLSPLPSPPEMMPTSPQS
PRDAELIAEAKLLRQHKGRLEARMQILEDHNKQLESQLHRLRQLLEQPQAEAKVNGTTVSSPSTSLQRSDSSQP
MLLRVVGSQTSDSMGEEDLLSPPQDTSTGLEEVMEQLNNSFPSSRGRNTPGKPMREDTM

### 6.0 REFERENCES

The following references, to the extent that they provide exemplary procedural or other details supplementary to those set forth herein, are specifically incorporated herein by reference.
U.S. Pat. No. 4,237,224, issued Dec. 2, 1980.
U.S. Pat. No. 4,554,101, issued Nov. 19, 1985.
U.S. Pat. No. 4,683,195, issued Jul. 28, 1987.
U.S. Pat. No. 4,683,202, issued Jul. 28, 1987.
U.S. Pat. No. 4,800,159, issued Jan. 24, 1989.
U.S. Pat. No. 4,883,750, issued Nov. 28, 1989.
U.S. Pat. No. 5,145,684, issued Sep. 8, 1992.
U.S. Pat. No. 5,399,363, issued Mar. 21, 1995.
U.S. Pat. No. 5,466,468, issued Nov. 14, 1995.
U.S. Pat. No. 5,543,158, issued Apr. 6, 1996.
U.S. Pat. No. 5,552,157, issued Sep. 3, 1996.
U.S. Pat. No. 5,565,213, issued Oct. 15, 1996.
U.S. Pat. No. 5,567,434, issued Oct. 22, 1996.
U.S. Pat. No. 5,602,306, issued Feb. 11, 1997.
U.S. Pat. No. 5,639,940, issued Jun. 17, 1997.
U.S. Pat. No. 5,641,515, issued Jun. 24, 1997.
U.S. Pat. No. 5,720,936, issued Feb. 24, 1998.
U.S. Pat. No. 5,738,868, issued Apr. 14, 1998.
U.S. Pat. No. 5,741,516, issued Apr. 21, 1998.
U.S. Pat. No. 5,795,587, issued Aug. 18, 1998.
Int. Pat. Appl. No. PCT/US87/00880.
Int. Pat. Appl. No. PCT/US89/01025.
Int. Pat. Appl. Publ. No. WO 88/10315.
Int. Pat. Appl. Publ. No. WO 89/06700.
Eur. Pat. Appl. Publ. No. EP 0329822.
Eur. Pat. Appl. Publ. No. 320,308.
Great Britain Appl. No. 2202328.
Allen and Choun, “Large unilamellar liposomes with low uptake into the reticuloendothelial system,” FEBS Lett., 223:42-46, 1987.
Anand, “For His Sick Kids, a Father Struggled to Develop a Cure,” The Wall Street Journal, 8-26-2003.
Angel, Bauman, Stein, Dellus, Rahmsdorf and Herrlich, “12-0-tetradecanoyl-phorbol-13-acetate induction of the human collagenase gene is mediated by an inducible enhancer element located in the 5′ flanking region,” Mol. Cell. Biol., 7:2256, 1987a.
Angel, Imagawa, Chiu, Stein, Imbra, Rahmsdorf, Jonat, Herrlich and Karin, “Phorbol ester-inducible genes contain a common cis element recognized by a TPA-modulated trans-acting factor,” Cell, 49:729, 1987b.
Atchison and Perry, “The role of the kappa enhancer and its binding factor NF-kappa B in the developmental regulation of kappa gene transcription,” Cell, 48:121, 1987.
Balazsovits et al., “Analysis of the effect of liposome encapsulation on the vesicant properties, acute and cardiac toxicities, and antitumor efficacy of doxorubicin,” Cancer Chemother. Pharmacol., 23:81-86, 1989.
Banerji, Olson and Schaffner, “A lymphocyte-specific cellular enhancer is located downstream of the joining region in immunoglobulin heavy-chain genes,” Cell, 35:729, 1983.
Banerji, Rusconi and Schaffner, “Expression of a β-globin gene is enhanced by remote SV40 DNA sequences,” Cell, 27:299, 1981.
Baranov et al., “Local and distant transfection of mdx muscle fibers with dystrophin and LacZ genes delivered in vivo by synthetic microspheres,” Gene Ther., 6:1406-14, 1999.
Baudhuin and Hers, “An electron microscopic and biochemical study of type II glycogenosis,” Lab. Invest., 13:1139-52, 1964.
Berkhout, Silverman and Jeang, “tat trans-activates the human immunodeficiency virus through a nascent RNA target,” Cell, 59:273, 1989.
Berns and Giraud, “Biology of adeno-associated virus,” Curr. Top Microbiol. Immunol., 218:1-23, 1996.
Berns and Linden, “The cryptic life style of adeno-associated virus,” Bioessays, 17:237-45, 1995.
Blanar, Baldwin, Flavell and Sharp, “A gamma-interferon-induced factor that binds the interferon response sequence of the MHC Class I gene, H-2Kb,” EMBO J., 8:1139, 1989.
Bodine and Ley, “An enhancer element lies 3′ to the Human A gamma globin gene,” EMBO J., 6:2997, 1987.
Boshart, Weber, Jahn, Dorsch-Hasler, Fleckenstein and Schaffner, “A very strong enhancer is located upstream of an immediate early gene of human cytomegalovirus,” Cell, 41:521, 1985.
Bosze, Thiesen and Charnay, “A transcriptional enhancer with specificity for erythroid cells is located in the long terminal repeat of the friend murine leukemia virus,” EMBO J., 5:1615, 1986.
Bouchard, MacKenzie, Radu, Hayashi, Peranteau, Chirmule and Flake, “Long-term transgene expression in cardiac and skeletal muscle following fetal administration of adenoviral or adeno-associated viral vectors in mice,” J. Gene Med., 5:941-50, 2003.
Braddock, Chambers, Wilson, Esnouf, Adams, Kingsman and Kingsman, “HIV-I tat activates presynthesized RNA in the nucleus,” Cell, 58:269, 1989.
Brooks and Faulkner, “Contractile properties of skeletal muscles from young, adult and aged mice,” J. Physiol., 404:71-82, 1988.
Bulla and Siddiqui, “The Hepatitis B virus enhancer modulates transcription of the Hepatitis B virus surface-antigen gene from an internal location,” J. Virol., 62:1437, 1986.
Buning et al., “Receptor targeting of adeno-associated virus vectors,” Gene Ther., 10:1142-51, 2003.
Caldovic and Hackett Jr., “Development of position-independent expression vectors and their transfer into transgenic fish,” Mol. Mar. Biol. Biotechnol., 4(1):51-61, 1995.
Campbell and Villarreal, “Functional analysis of the individual enhancer core sequences of polyoma virus: cell-specific uncoupling of DNA replication from transcription,” Mol. Cell. Biol., 8:1993, 1988.
Campere and Tilghman, “Postnatal repression of the α-fetoprotein gene is enhancer independent,” Genes and Dev., 3:537, 1989.
Campo, Spandidos, Lang and Wilkie, “Transcriptional control signals in the genome of bovine papilloma virus Type 1,” Nature, 303:77, 1983.
Capecchi, “High efficiency transformation by direct microinjection of DNA into cultured mammalian cells,” Cell, 22(2):479-488, 1980.
Carver, Dalrymple, Wright, Cottom, Reeves, Gibson, Keenan, Barrass, Scott, Colman, et al., “Transgenic livestock as bioreactors: stable expression of human α-1-antitrypsin by a flock of sheep,” Biotechnology NY, 11(11): 1263-1270, 1993.
Celander and Haseltine, “Glucocorticoid regulation of murine leukemia virus transcription elements is specified by determinants within the viral enhancer region,” J. Virology, 61:269, 1987.
Celander, Hsu and Haseltine, “Regulatory elements within the murine leukemia virus enhancer regions mediate glucocorticoid responsiveness,” J. Virology, 62:1314, 1988.
Champion, Georgakopoulos, Haldar, Wang, Wang and Kass, “Robust adenoviral and adeno-associated viral gene transfer to the in vivo murine heart: application to study of phospholamban physiology,” Circulation, 108:2790-97, 2003.
Chandler, Maler and Yamamoto, “DNA sequences bound specifically by glucocorticoid receptor in vitro render a heterlogous promoter hormone responsive in vivo,” Cell, 33:489, 1983.
Chandran, Roy, Mishra, “Recent trends in drug delivery systems: liposomal drug delivery system—preparation and characterization,” Indian J. Exp. Biol., 35(8):801-809, 1997.
Chang, Erwin and Lee, “Glucose-regulated protein (GRP94 and GRP78) genes share common regulatory domains and are coordinately regulated by common trans-acting factors,” Mol. Cell. Biol., 9:2153, 1989.
Chao, Ravinowitz, Li, Samulski and Walsh, “Several log increase in therapeutic transgene delivery by distinct adeno-associated viral serotype vectors,” Mol. Ther., 2:619-23, 2000.
Chao, Monahan, Liu, Samulski and Walsh, “Sustained and complete phenotype correction of hemophilia B mice following intramuscular injection of AAV1 serotype vectors,” Mol. Ther., 4:217-22, 2001.
Chatterjee, Lee, Rentoumis and Jameson, “Negative regulation of the thyroid-stimulating hormone a gene by thyroid hormone: Receptor interaction adjacent to the TATA box,” Proc. Natl. Acad. Sci. U.S.A., 86:9114, 1989.
Chen and Okayama, “High-efficiency transformation of mammalian cells by plasmid DNA,” Mol. Cell. Biol., 7:2745-2752, 1987.
Choi, Chen, Kriegler and Roninson, “An altered pattern of cross-resistance in multi-drug-resistant human cells results from spontaneous mutations in the MDR-1 (P-glycoprotein) gene,” Cell, 53:519, 1988.
Chu, Thistlethwaite, Sullivan, Grifman and Weitzman, “Gene delivery to the mammalian heart using AAV vectors,” Methods Mol. Biol., 246:213-24, 2004.
Clark, Sferra and Johnson, “Recombinant adeno-associated viral vectors mediate long-term transgene expression in muscle,” Hum. Gene Ther., 8:659-69, 1997.
Cohen, Walter and Levinson, “A repetitive sequence element 3′ of the human c-Ha-ras1 gene has enhancer activity,” J. Cell. Physiol., 5:75, 1987.
Costa, Lai, Grayson and Darnell, “The cell-specific enhancer of the mouse transthyretin (prealbumin) gene binds a common factor at one site and a liver-specific factor(s) at two other sites,” Mol. Cell. Biol., 8:81, 1988.
Coune, “Liposomes as drug delivery system in the treatment of infectious diseases: potential applications and clinical experience,” Infection, 16(3):141-147, 1988.
Couvreur et al., “Nanocapsules, a new lysosomotropic carrier,” FEBS Lett., 84:323-326, 1977.
Couvreur et al., “Tissue distribution of antitumor drugs associated with polyalkylcyanoacrylate nanoparticles,” J. Pharm. Sci., 69(2):199-202, 1980.
Couvreur, “Polyalkyleyanoacrylates as colloidal drug carriers,” Crit. Rev. Ther. Drug Carrier Syst., 5:1-20, 1988.
Cozzi, Tucker, Langford, Pino-Chavez, Wright, O'Connell, Young, Lancaster, McLanghlin, Hunt, Bordin, White, “Characterization of pigs transgenic for human decay-accelerating factor,” Transplantation, 64(10):1383-1392, 1997.
Cripe, Haugen, Turk, Tabatabai, Schmid, Durst, Gissmann, Roman and Turek, “Transcriptional regulation of the human papilloma virus-16 E6-E7 promoter by a keratinocyte-dependent enhancer, and by viral E2 trans-activator and repressor gene products: Implications for cervical carcinogenesis,” EMBO J., 6:3745, 1987.
Criswell, Powers, Herb and Dodd, “Mechanism of specific force deficit in the senescent rat diaphragm,” Respir. Physiol., 107:149-55, 1997.
Croyle, Cheng and Wilson, “Development of formulations that enhance physical stability of viral vectors for gene therapy,” Gene Ther., 8:1281-90, 2001.
Culotta and Hamer, “Fine mapping of a mouse metallothionein gene metal-response element,” Mol. Cell. Biol., 9:1376, 1989.
Curiel, Agarwal, Wagner, Cotten, “Adenovirus enhancement of transferrin-polylysine-mediated gene delivery,” Proc. Natl. Acad. Sci. USA, 88(19):8850-8854, 1991.
Daly, Ohlemiller, Roberts, Vogler and Sands, “Prevention of systemic clinical disease in MPS VII mice following AAV-mediated neonatal gene transfer,” Gene Ther., 8:1291-98, 2001.
Dandolo, Blangy and Kamen, “Regulation of polyma virus transcription in murine embryonal carcinoma cells,” J. Virology, 47:55, 1983.
De Villiers, Schaffner, Tyndall, Lupton and Kamen, “Polyoma virus DNA replication requires an enhancer,” Nature, 312:242, 1984.
Deschamps, Meijlink and Verma, “Identification of a transcriptional enhancer element upstream from the proto-oncogene Fos,” Science, 230:1174, 1985.
Douglas, Davis, Illum, “Nanoparticles in drug delivery,” Crit. Rev. Ther. Drug Carrier Syst., 3(3):233-261, 1987.
Ebert, Selgrath, DiTullio, Denman, Smith, Memon, Schindler, Monastersky, Vitale, Gordon, “Transgenic production of a variant of human tissue-type plasminogen activator in goat milk: generation of transgenic goats and analysis of expression,” Biotechnology NY, 9(9):835-838, 1991.
Edbrooke, Burt, Cheshire and Woo, “Identification of cis-acting sequences responsible for phorbol ester induction of human serum amyloid a gene expression via a nuclear-factor-κB-like transcription factor,” Mol. Cell. Biol., 9:1908, 1989.
Edlund, Walker, Barr and Rutter, “Cell-specific expression of the rat insulin gene: Evidence for role of two distinct 5′ flanking elements,” Science, 230:912, 1985.
Eisen, Spellman, Brown and Botstein, “Cluster analysis and display of genome-wide expression patterns,” Proc. Natl. Acad. Sci. USA, 95:14863-68, 1998.
Faller and Baltimore, “Liposome encapsulation of retrovirus allows efficient super infection of resistant cell lines,” J. Virol., 49(1):269-272, 1984.
Fallon, “Transgenic insect cells: mosquito cell mutants and the dihydrofolate reductase gene,” Cytotechnology, 20(1-3):23-31, 1996.
Fechheimer et al., “Transfection of mammalian cells with plasmid DNA by scrape loading and sonication loading,” Proc. Natl. Acad. Sci. USA, 84:8463-8467, 1987.
Feng and Holland, “HIV-I tat trans-activation requires the loop sequence within tar,” Nature, 334:6178, 1988.
Firak and Subramanian, “Minimal transcription enhancer of simian virus 40 is a 74-base-pair sequence that has interacting domains,” Mol. Cell. Biol., 6:3667, 1986.
Fisher et al., “Recombinant adeno-associated virus for muscle directed gene therapy,” Nat. Med., 3:306-12, 1997.
Foecking and Hofstetter, “Powerful and versatile enhancer-promoter unit for mammalian expression vectors,” Gene, 45(1):101-105, 1986.
Fraites, Jr., Schleissing, Shanely, Walter, Cloutier, Zolotukhin, Pauly, Raben, Plotz, Powers, Kessler and Byrne, “Correction of the enzymatic and functional deficits in a model of Pompe disease using adeno-associated virus vectors,” Mol. Ther., 5:571-78, 2002.
Fraites, Germain, Cahill, Cloutier, Porvasnik, Zolotukhin, Walter and Byrne, “Gene replacement therapy for glycogen storage disease type II with recombinant adeno-associated virus serotype 1 (AAV1) vectors,” Circulation, 106:11-127, 2003.
Fraley et al., “Entrapment of a bacterial plasmid in phospholipid vesicles: Potential for gene transfer,” Proc. Natl. Acad. Sci. USA, 76:3348-3352, 1979.
Franz, Mueller, Haartong, Frey, Katus, “Transgenic animal models: new avenues in cardiovascular physiology,” J. Mol. Med., 75(2):115-119, 1997.
Fresta and Puglisi, “Application of liposomes as potential cutaneous drug delivery systems. In vitro and in vivo investigation with radioactively labelled vesicles,” J. Drug Target, 4(2):95-101, 1996.
Frohman, Downs, Kashio, Brinster, “Tissue distribution and molecular heterogeneity of human growth hormone-releasing factor in the transgenic mouse,” Endocrinology, 127(5):2149-2156, 1990.
Fromm, Taylor, Walbot, “Expression of genes transferred into monocot and dicot plant cells by electroporation,” Proc. Natl. Acad. Sci. USA, 82(17):5824-5828, 1985.
Fujita, Shibuya, Hotta, Yamanishi and Taniguchi, “Interferon-β gene regulation: tandemly repeated sequences of a synthetic 6-bp oligomer function as a virus-inducible enhancer,” Cell, 49:357, 1987.
Gabizon and Papahadjopoulos, “Liposomes formulations with prolonged circulation time in blood and enhanced uptake by tumors,” Proc. Natl. Acad. Sci. USA, 85:6949-6953, 1988.
Gao, Alvira, Wang, Calcedo, Johnston and Wilson, “Novel adeno-associated viruses from rhesus monkeys as vectors for human gene therapy,” Proc. Natl. Acad. Sci. USA, 99:11854-59, 2002.
Gao, Alvira, Somanathan, Lu, Vandenberghe, Rux, Calcedo, Sanmiguel, Abbas and Wilson, “Adeno-associated viruses undergo substantial evolution in primates during natural infections,” Proc. Natl. Acad. Sci. USA, 100:6081-86, 2003.
Gilles, Morris, Oi and Tonegawa, “A tissue-specific transcription enhancer element is located in the major intron of a rearranged immunoglobulin heavy-chain gene,” Cell, 33:717, 1983.
Gloss, Bernard, Seedorf and Klock, “The upstream regulatory region of the human papilloma virus-16 contains an E2 protein-independent enhancer which is specific for cervical carcinoma cells and regulated by glucocorticoid hormones,” EMBO J., 6:3735, 1987.
Godbout, Ingram and Tilghman, “Fine-structure mapping of the three mouse α-fetoprotein gene enhancers,” Mol. Cell. Biol., 8:1169, 1988.
Goodbourn and Maniatis, “Overlapping positive and negative regulatory domains of the human β-interferon gene,” Proc. Natl. Acad. Sci. USA, 85:1447, 1988.
Goodbourn, Burstein and Maniatis, “The human β-interferon gene enhancer is under negative control,” Cell, 45:601, 1986.
Gopal, “Gene transfer method for transient gene expression, stable transfection, and cotransfection of suspension cell cultures,” Mol. Cell Biol., 5:1188-1190, 1985.
Gossen and Bujard, “Tight control of gene expression in mammalian cells by tetracycline-responsive promoters,” Proc. Natl. Acad. Sci. USA, 89:5547-51, 1992.
Graham and van der Eb, “Transformation of rat cells by DNA of human adenovirus 5,” Virol., 54(2):536-539, 1973.
Greene, Böhnlein and Ballard, “HIV-1, and normal T-cell growth: Transcriptional strategies and surprises,” Immunol. Today, 10:272, 1989.
Gregorevic, Blankinship, Allen, Meuse, Han, Oakley and Chamberlain, “Systemic gene transfer to striated muscles using rAAV6 vectors,” Mol. Ther., 9:S274, 2004.
Grimm, Kay and Kleinschmidt, “Helper virus-free, optically controllable, and two-plasmid-based production of adeno-associated virus vectors of serotypes 1 to 6,” Mol. Ther., 7:839-50, 2003.
Grosschedl and Baltimore, “Cell-type specificity of immunoglobulin gene expression is regulated by at least three DNA sequence elements,” Cell, 41:885, 1985.
Harland and Weintraub, “Translation of mRNA injected into Xenopus oocytes is specifically inhibited by antisense RNA,” J. Cell Biol., 101(3):1094-1099, 1985.
Haskell and Bowen, “Efficient production of transgenic cattle by retroviral infection of early embryos,” Mol. Reprod. Dev., 40(3):386-390, 1995.
Haslinger and Karin, “Upstream promoter element of the human metallothionein-II gene can act like an enhancer element,” Proc. Natl. Acad. Sci. USA, 82:8572, 1985.
Hauber and Cullen, “Mutational analysis of the trans-activation-responsive region of the human immunodeficiency virus Type I long terminal repeat,” J. Virol., 62:673, 1988.
Hauck and Xiao, “Characterization of tissue tropism determinants of adeno-associated virus type 1,” J. Virol., 77:2768-74, 2003.
Heath and Martin, “The development and application of protein-liposome conjugation techniques,” Chem. Phys. Lipids, 40:347-358, 1986.
Heath et al., “Liposome-mediated delivery of pteridine antifolates to cells: in vitro potency of methotrexate and its a and gamma substituents,” Biochim. Biophys. Acta, 862:72-80, 1986.
Hen, Borrelli, Fromental, Sassone-Corsi and Chambon, “A mutated polyoma virus enhancer which is active in undifferentiated embryonal carcinoma cells is not repressed by adenovirus-2 E1A products,” Nature, 321:249, 1986.
Henry-Michelland et al., “Attachment of antibiotics to nanoparticles; Preparation, drug-release and antimicrobial activity in vitro,” Int. J. Pharm., 35:121-127, 1987.
Hensel, Meichle, Pfizenmaier and Kronke, “PMA-responsive 5′ flanking sequences of the human TNF gene,” Lymphokine Res., 8:347, 1989.
Herr and Clarke, “The SV40 enhancer is composed of multiple functional elements that can compensate for one another,” Cell, 45:461, 1986.
Hers, “Alpha-glucosidase deficiency in generalized glycogen storage disease (Pompe's disease),” Biochem. J., 86:11, 1963.
Hirochika, Browker and Chow, “Enhancers and trans-acting E2 transcriptional factors of papilloma viruses,” J. Virol., 61:2599, 1987.
Hirsch, Gaugler, Deagostini-Bauzin, Bally-Cuif and Gordis, “Identification of positive and negative regulatory elements governing cell-type-specific expression of the neural-cell-adhesion-molecule gene,” Mol. Cell. Biol., 10:1959, 1990.
Hirschhorn and Reuser, “Glycogen storage disease II: acid-alpha glucosidase (acid maltase) deficiency,” In METABOLIC BASIS OF INHERITED DISEASE. Scriver, Beaudet, Sly and Valle (eds.), McGraw Hill, New York, 2000.
Holbrook, Gulino and Ruscetti, “cis-acting transcriptional regulatory sequences in the Gibbon ape leukemia virus (GALV) long terminal repeat,” Virology, 157:211, 1987.
Horlick and Benfield, “The upstream muscle-specific enhancer of the rat muscle creatine kinase gene is composed of multiple elements,” Mol. Cell. Biol., 9:2396, 1989.
Huang, Ostrowski, Berard and Hagar, “Glucocorticoid regulation of the Ha-MuSV p21 gene conferred by sequences from mouse mammary tumor virus,” Cell, 27:245, 1981.
Hwang, Lim and Chae, “Characterization of the s-phase-specific transcription regulatory elements in a DNA-replication-independent testis-specific H2B (TH2B) histone gene,” Mol. Cell. Biol., 10:585, 1990.
Imagawa, Chiu and Karin, “Transcription factor AP-2 mediates induction by two different signal-transduction pathways: Protein kinase C and cAMP,” Cell, 51:251, 1987.
Imaizumi et al., “Liposome-entrapped superoxide dismutase ameliorates infarct volume in focal cerebral ischemia,” Acta. Neurochirurgica Suppl., 51:236-238, 1990b.
Imaizumi et al., “Liposome-entrapped superoxide dismutase reduces cerebral infarction in cerebral ischemia in rats,” Stroke, 21(9):1312-1317, 1990a.
Imbra and Karin, “Phorbol ester induces the transcriptional stimulatory activity of the SV40 enhancer,” Nature, 323:555, 1986.
Imler, Lemaire, Wasvlyk and Waslyk, “Negative regulation contributes to tissue specificity of the immunoglobulin heavy-chain enhancer,” Mol. Cell. Biol., 7:2558, 1987.
Imperiale and Nevins, “Adenovirus 5 E2 transcription unit: An E1A-inducible promoter with an essential element that functions independently of position or orientation,” Mol. Cell. Biol., 4:875, 1984.
Iwatate, Gu, Dieterle, Iwanaga, Peterson, Hoshijima, Chien and Ross, “In vivo high-efficiency transcoronary gene delivery and Cre-LoxP gene switching in the adult mouse heart,” Gene Ther., 10:1814-20, 2003.
Jakobovits, Smith, Jakobovits and Capon, “A discrete element 3′ of human immunodeficiency virus 1 (HIV-1) and HIV-2 mRNA initiation sites mediates transcriptional activation by an HIV trans-activator,” Mol. Cell. Biol., 8:2555, 1988.
Jameel and Siddiqui, “The human Hepatitis B virus enhancer requires transacting cellular factor(s) for activity,” Mol. Cell. Biol., 6:710, 1986.
Jaynes, Johnson, Buskin, Gartside and Hauschka, “The muscle creatine kinase gene is regulated by multiple upstream elements, including a muscle-specific enhancer,” Mol. Cell. Biol., 8:62, 1988.
Johnson, Wold and Hauschka, “Muscle creatine kinase sequence elements regulating skeletal and cardiac muscle expression in transgenic mice,” Mol. Cell. Biol., 9:3393, 1989a.
Jooss and Chirmule, “Immunity to adenovirus and adeno-associated viral vectors: implications for gene therapy,” Gene Ther., 10:955-63, 2003.
Kadesch and Berg, “Effects of the position of the simian virus 40 enhancer on expression of multiple transcription units in a single plasmid,” Mol. Cell. Biol., 6:2593, 1986.
Karin, Haslinger, Heguy, Dietlin and Cooke, “Metal-responsive elements act as positive modulators of human metallothionein-IIa enhancer activity,” Mol. Cell. Biol., 7:606, 1987.
Katinka, Vasseur, Montreau, Yaniv and Blangy, “Polyoma DNA sequences involved in the control of viral gene expression in murine embryonal carcinoma cells,” Nature, 290:720, 1981.
Katinka, Yaniv, Vasseur and Blangy, “Expression of polyoma early functions in mouse embryonal carcinoma cells depends on sequence rearrangements in the beginning of the late region,” Cell, 20:393, 1980.
Kawamoto et al., “AAV6 vectors promote efficient transduction and gene dissemination through myocardium in vivo,” Mol. Ther., 7:S228, 2003.
Kawamoto, Makino, Niw, Sugiyama, Kimura, Anemura, Nakata and Kakunaga, “Identification of the human β-actin enhancer and its binding factor,” Mol. Cell. Biol., 8:267, 1988.
Kessler, Podsakoff, Chen, McQuiston, Colosi, Matelis, Kurtzman and Byrne, “Gene delivery to skeletal muscle results in sustained expression and systemic delivery of a therapeutic protein,” Proc. Natl. Acad. Sci. USA, 93:14082-87, 1996.
Kiledjian, Su and Kadesch, “Identification and characterization of two functional domains within the murine heavy-chain enhancer,” Mol. Cell. Biol., 8:145, 1988.
Klamut, Gangopadyhay, Worton and Ray, “Molecular and functional analysis of the muscle-specific promoter region of the Duchenne muscular dystrophy gene,” Mol. Cell. Biol., 10:193, 1990.
Klein, Wolf, Wu, Sanford, “High-velocity microprojectiles for delivering nucleic acids into living cells. 1987,” Biotechnology, 24:384-386, 1992.
Koch, Benoist and Mathis, “Anatomy of a new B-cell-specific enhancer,” Mol. Cell. Biol., 9:303, 1989.
Kriegler and Botchan, “A retrovirus LTR contains a new type of eukaryotic regulatory element,” In: Eukaryotic Viral Vectors, Gluzman, Ed., Cold Spring Harbor, Cold Spring Harbor Laboratory, N.Y., 1982.
Kriegler and Botchan, “Enhanced transformation by a simian virus 40 recombinant virus containing a Harvey murine sarcoma virus long terminal repeat,” Mol. Cell. Biol. 3:325, 1983.
Kriegler, Perez and Botchan, “Promoter substitution and enhancer augmentation increases the penetrance of the SV40 a gene to levels comparable to that of the Harvey murine sarcoma virus Ras gene in morphologic transformation,” In: Gene Expression, Hamer and Rosenberg, Eds., New York, Alan R. Liss, 1983.
Kriegler, Perez, Defay, Albert and Liu, “A novel form of TNF/cachectin is a cell-surface cytotoxix transmembrane protein: Ramifications for the complex physiology of TNF,” Cell, 53:45, 1988.
Kriegler, Perez, Hardy and Botchan, “Transformation mediated by the SV40 T antigens: separation of the overlapping SV40 early genes with a retroviral vector,” Cell, 38:483, 1984a.
Kriegler, Perez, Hardy and Botchan, “Viral integration and early gene expression both affect the efficiency of SV40 transformation of murine cells: Biochemical and biological characterization of an SV40 retrovirus,” In: Cancer Cells 2/Oncogenes and Viral Genes, Van de Woude, Levine, Topp and Watson, Eds., Cold Spring Harbor, Cold Spring Harbor Laboratory, 1984b.
Kuby, In: Immunology, 2nd Edition. W.H. Freeman & Company, New York, 1994.
Kuhl, De La Fuenta, Chaturvedi, Parinool, Ryals, Meyer and Weissman, “Reversible silencing of enhancers by sequences derived from the human IFN-α promoter,” Cell, 50:1057, 1987.
Kunz, Zimmerman, Heisig and Heinrich, “Identification of the promoter sequences involved in the Interleukin-6-dependent expression of the rat α-2-macroglobulin gene,” Nucl. Acids Res., 17:1121, 1989.
Kwoh, Davis, Whitfield, Chappelle, DiMichele, Gingeras, “Transcription-based amplification system and detection of amplified human immunodeficiency virus type 1 with a bead-based sandwich hybridization format,” Proc. Natl. Acad. Sci. USA, 86(4):1173-1177, 1989.
Kyte and Doolittle, “A simple method for displaying the hydropathic character of a protein,” J. Mol. Biol., 157(1):105-132, 1982.
Larsen, Harney and Moore, “Repression mediates cell-type-specific expression of the rat growth hormone gene,” Proc. Natl. Acad. Sci. U.S.A., 83:8283, 1986.
Lasic, “Novel applications of liposomes,” Trends Biotechnol., 16(7):307-321, 1998.
Laspia, Rice and Mathews, “HIV-1 tat protein increases transcriptional initiation and stabilizes elongation,” Cell, 59:283, 1989.
Latimer, Berger and Baumann, “Highly conserved upstream regions of the α₁-antitrypsin gene in two mouse species govern liver-specific expression by different mechanisms,” Mol. Cell. Biol., 10:760, 1990.
Lee et al., “Functional analysis of the steroid hormone control region of mouse mammary tumor virus,” Nucleic Acids Res., 12(10):4191-4206, 1984.
Lee, Mulligan, Berg and Ringold, “Glucocorticoids regulate expression of dihydrofolate reductase cDNA in mouse mammary tumor virus chimaeric plasmids,” Nature, 294:228, 1981.
Levinson, Khoury, VanDeWoude and Gruss, “Activation of SV40 genome by 72-base-pair tandem repeats of Moloney sarcoma virus,” Nature, 295:79, 1982.
Li, Wang, Qian, Chen, Zhu and Xiao, “Efficient and long-term intracardiac gene transfer in delta-sarcoglycan-deficiency hamster by adeno-associated virus-2 vectors,” Gene Ther., 10:1807-13, 2003.
Limberis, Gao, Weiner, Wang, Bell and Wilson, “A novel AAV vector for the treatment of cystic fibrosis airway disease,” Mol. Ther., 9:S262-63, 2004.
Lin, Cross, Halden, Dragos, Toledano and Leonard, “Delineation of an enhancer like positive regulatory element in the interleukin-2 receptor α-chain gene,” Mol. Cell. Biol., 10:850, 1990.
Liu, Nishikawa, Clemens and Huang, “Transfer of full-length Dmd to the diaphragm muscle of Dmd(mdx/mdx) mice through systemic administration of plasmid DNA,” Mol. Ther., 4:45-51, 2001.
Lopez-Berestein et al., “Liposomal amphotericin B for the treatment of systemic fungal infections in patients with cancer: a preliminary study” J. Infect. Dis., 2151:704, 1985a.
Lopez-Berestein et al., “Protective effect of liposomal-amphotericin B against C. albicans infection in mice,” Cancer Drug Delivery, 2:183, 1985b.
Luria, Gross, Horowitz and Givol, “Promoter enhancer elements in the rearranged α-chain gene of the human T-cell receptor,” EMBO J., 6:3307, 1987.
Lusky and Botchan, “Transient replication of bovine papilloma virus Type 1 plasmids: cis and trans requirements,” Proc. Natl. Acad. Sci. U.S.A., 83:3609, 1986.
Lusky, Berg, Weiher and Botchan, “Bovine papilloma virus contains an activator of gene expression at the distal end of the early transcription unit,” Mol. Cell. Biol. 3:1108, 1983.
Mah, Byrne and Flotte, “Virus-based gene delivery systems,” Clin. Pharmacokinet., 41:901-11, 2002.
Mah et al., “Improved method of recombinant AAV2 delivery for systemic targeted gene therapy,” Mol. Ther., 6:106-12, 2002.
Mah, Sarkar, Zolotukhin, Schleissing, Xiao, Kazazian and Byrne, “Dual vectors expressing murine factor VIII result in sustained correction of hemophilia A mice,” Hum. Gene Ther., 14:143-52, 2003.
Mah, Fraites, Jr., Cresawn, Zolotukhin, Lewis and Byrne, “A new method for recombinant adeno-associated virus vector delivery to murine diaphragm,” Mol. Ther., 9:458-63, 2004.
Majors and Varmus, “A small region of the mouse mammary tumor virus long terminal repeat confers glucocorticoid hormone regulation on a linked heterologous gene,” Proc. Natl. Acad. Sci. U.S.A., 80:5866, 1983.
Maloy et al., In: Microbial Genetics, 2nd Edition, Jones and Barlett Publishers, Boston, Mass., 1994.
Maniatis et al., “Molecular Cloning: a Laboratory Manual,” Cold Spring Harbor Laboratory, Cold Spring Harbor, N.Y., 1982.
Manno, Chew, Hutchison, Larson, Herzog, Arruda, Tai, Ragni, Thompson, Ozelo, Couto, Leonard, Johnson, McClelland, Scallan, Skarsgard, Flake, Kay, High and Glader, “AAV-mediated factor IX gene transfer to skeletal muscle in patients with severe hemophilia B,” Blood, 101:2963-72, 2003.
March, Madison and Trapnell, “Pharmacokinetics of adenoviral vector-mediated gene delivery to vascular smooth muscle cells: modulation by poloxamer 407 and implications for cardiovascular gene therapy,” Hum. Gene Ther. 6:41-53, 1995.
Margalit, “Liposome-mediated drug targeting in topical and regional therapies,” Crit. Rev. Ther. Drug Carrier Syst., 12(2-3):233-261, 1995.
McNeall, Sanchez, Gray, Chesterman and Sleigh, “Hyperinducible gene expression from a metallotionein promoter containing additional metal-responsive elements,” Gene, 76:81, 1989.
Miksicek, Heber, Schmid, Danesch, Posseckert, Beato and Schutz, “Glucocorticoid responsiveness of the transcriptional enhancer of Moloney murine sarcoma virus,” Cell, 46:203, 1986.
Mingozzi, Schuttrumpf, Arruda, Liu, Liu, High, Xiao and Herzog, “Improved hepatic gene transfer by using an adeno-associated virus serotype 5 vector,” J. Virol., 76:10497-502, 2002.
Mordacq and Linzer, “Co-localization of elements required for phorbol ester stimulation and glucocorticoid repression of proliferin gene expression,” Genes and Dev., 3:760, 1989.
Moreau, Hen, Wasylyk, Everett, Gaub and Chambon, “The SV40 base-repair repeat has a striking effect on gene expression both in SV40 and other chimeric recombinants,” Nucl. Acids Res., 9:6047, 1981.
Mori and Fukatsu, “Anticonvulsant effect of DN-1417 a derivative of thyrotropin-releasing hormone and liposome-entrapped DN-1417 on amygdaloid-kindled rats,” Epilepsia, 33(6):994-1000, 1992.
Moufarrej and Bertorini, “Respiratory insufficiency in adult-type acid maltase deficiency,” South. Med. J., 86:560-67, 1993.
Muesing, Smith and Capon, “Regulation of mRNA accumulation by a human immunodeficiency virus trans-activator protein,” Cell, 48:691, 1987.
Muller et al., “Efficient transfection and expression of heterologous genes in PC12 cells,” Cell, Biol., 9(3):221-229, 1990.
Muller et al., “Random peptide libraries displayed on adeno-associated virus to select for targeted gene therapy vectors,” Nat. Biotechnol., 21:1040-46, 2003.
Muzyczka, “Use of adeno-associated virus as a general transduction vector for mammalian cells,” Curr Top Microbiol Immunol., 158:97-129, 1992.
Ng, Gunning, Liu, Leavitt and Kedes, “Regulation of the human β-actin promoter by upstream and intron domains,” Nuc. Acids Res., 17:601, 1989.
Nicolau and Gersonde, “Incorporation of inositol hexaphosphate into intact red blood cells, I. fusion of effector-containing lipid vesicles with erythrocytes,” Naturwissenschaften (Germany), 66(11):563-566, 1979.
Nicolau and Sene, “Liposome-mediated DNA transfer in eukaryotic cells,” Biochem. Biophys. Acta, 721:185-190, 1982.
Ohara, Dort, Gilbert, “One-sided polymerase chain reaction: the amplification of cDNA,” Proc. Natl. Acad. Sci. USA, 86(15):5673-5677, 1989.
Ondek, Sheppard and Herr, “Discrete elements within the SV40 enhancer region display different cell-specific enhancer activities,” EMBO J., 6:1017, 1987.
Ono, Hirose, Miyazaki, Yamamoto, Matsumoto, “Transgenic medaka fish bearing the mouse tyrosinase gene: expression and transmission of the transgene following electroporation of the orange-colored variant,” Pigment Cell Res., 10(3):168-175, 1997.
Ornitz, Hammer, Davison, Brinster and Palmiter, “Promoter and enhancer elements from the rat elastase I gene function independently of each other and of heterologous enhancers,” Mol. Cell. Biol., 7:3466, 1987.
Palmiter, Chen and Brinster, “Differential regulation of metallothionein-thymidine kinase fusion genes in transgenic mice and their offspring,” Cell, 29:701, 1982.
Pech, Rao, Robbins and Aaronson, “Functional identification of regulatory elements within the promoter region of platelet-derived growth factor 2,” Mol. Cell. Biol., 9:396, 1989.
Perabo et al., “In vitro selection of viral vectors with modified tropism: the adeno-associated virus display,” Mol. Ther., 8:151-57, 2003.
Perez-Stable and Constantini, “Roles of fetal γ-globin promoter elements and the adult β-globin 3′ enhancer in the stage-specific expression of globin genes,” Mol. Cell. Biol., 10:1116, 1990.
Petrof, Acsadi, Jani, Massie, Bourdon, Matusiewicz, Yang, Lochmuller and Karpati, “Efficiency and functional consequences of adenovirus-mediated in vivo gene transfer to normal and dystrophic (mdx) mouse diaphragm,” Am. J. Respir. Cell Mol. Biol., 13:508-17, 1995.
Petrof, “Respiratory muscles as a target for adenovirus-mediated gene therapy,” Eur. Respir. J., 11:492-97, 1998.
Picard and Schaffner, “A Lymphocyte-specific enhancer in the mouse immunoglobulin kappa gene,” Nature, 307:83, 1984.
Pikul et al., “In vitro killing of melanoma by liposome-delivered intracellular irradiation, Arch. Surg., 122(12):1417-1420, 1987.
Pinkert, Ornitz, Brinster and Palmiter, “An albumin enhancer located 10 kb upstream functions along with its promoter to direct efficient, liver-specific expression in transgenic mice,” Genes and Dev., 1:268, 1987.
Pinto-Alphandary, Balland, Couvreur, “A new method to isolate polyalkylcyanoacrylate nanoparticle preparations,” J. Drug Target, 3(2):167-169, 1995.
Pinto-Sietsma and Paul, “Transgenic rats as models for hypertension,” J. Hum. Hypertens., 11(9):577-581, 1997.
Ponnazhagan, Mahendra, Kumar, Thompson and Castillas, “Conjugate-based targeting of recombinant adeno-associated virus type 2 vectors by using avidin-linked ligands,” J. Virol., 76:12900-07, 2002.
Ponta, Kennedy, Skroch, Hynes and Groner, “Hormonal response region in the mouse mammary tumor virus long terminal repeat can be dissociated from the proviral promoter and has enhancer properties,” Proc. Natl. Acad. Sci. U.S.A., 82:1020, 1985.
Porton, Zaller, Lieberson and Eckhardt, “Immunoglobulin heavy-chain enhancer is required to maintain transfected γ2A gene expression in a pre-B-cell line,” Mol. Cell. Biol., 10:1076, 1990.
Potter et al., “Enhancer-dependent expression of human κ immunoglobulin genes introduced into mouse pre-B lymphocytes by electroporation,” Proc. Natl. Acad. Sci. USA, 81:7161-7165, 1984.
Prokop and Bajpai, “Recombinant DNA Technology I,” Conference on Progress in Recombinant DNA Technology Applications, Potosi, Mich., Jun. 3-8, 1990, Ann. N.Y Acad. Sci., 646:1-383, 1991.
Queen and Baltimore, “Immunoglobulin gene transcription is activated by downstream sequence elements,” Cell, 35:741, 1983.
Quinn, Farina, Gardner, Krutzsch and Levens, “Multiple components are required for sequence recognition of the AP1 site in the Gibbon ape leukemia virus enhancer,” Mol. Cell. Biol., 9:4713, 1989.
Quintanar-Guerrero, Allemann, Doelker, Fessi, “Preparation and characterization of nanocapsules from preformed polymers by a new process based on emulsification-diffusion technique,” Phamr. Res., 15(7):1056-1062, 1998.
Raben, Nagaraju, Lee, Kessler, Byrne, Lee, LaMarca, King, Ward, Sauer and Plotz, “Targeted disruption of the acid alpha-glucosidase gene in mice causes an illness with critical features of both infantile and adult human glycogen storage disease type II,” J. Biol. Chem., 273:19086-92, 1998.
Raben, Lu, Nagaraju, Rivera, Lee, Yan, Byrne, Meikle, Umapathysivam, Hopwood and Plotz, “Conditional tissue-specific expression of the acid alpha-glucosidase (GAA) gene in the GAA knockout mice: implications for therapy,” Hum. Mol. Genet., 10:2039-47, 2001.
Raben, Jatkar, Lee, Lu, Dwivedi, Nagaraju and Plotz, “Glycogen stored in skeletal but not in cardiac muscle in acid alpha-glucosidase mutant (Pompe) mice is highly resistant to transgene-encoded human enzyme,” Mol. Ther., 6:601-08, 2002.
Raben, Nagaraju, Lee, Lu, Rivera, Jatkar, Hopwood and Plotz, “Induction of tolerance to a recombinant human enzyme, acid alpha-glucosidase, in enzyme deficient knockout mice,” Transgenic Res., 12:171-78, 2003.
Rabinowitz and Samulski, “Adeno-associated virus expression systems for gene transfer,” Curr. Opin. Biotechnol., 9:470-75, 1998.
Rabinowitz et al., “Cross-packaging of a single adeno-associated virus (AAV) type 2 vector genome into multiple AAV serotypes enables transduction with broad specificity,” J. Virol., 76:791-801, 2002.
Redondo, Hata, Brocklehurst and Krangel, “A T-cell-specific transcriptional enhancer within the human T-cell receptor δ locus,” Science, 247:1225, 1990.
Reisman and Rotter, “Induced expression from the Moloney murine leukemia virus long terminal repeat during differentiation of human myeloid cells is mediated through its transcriptional enhancer,” Mol. Cell. Biol., 9:3571, 1989.
Renneisen, “Inhibition of expression of human immunodeficiency virus-1 in vitro by antibody-targeted liposomes containing antisense RNA to the env region,” J. Biol. Chem., 265(27):16337-16342, 1990.
Resendez Jr., Wooden and Lee, “Identification of highly conserved regulatory domains and protein-binding sites in the promoters of the rat and human genes encoding the stress-inducible 78-kilodalton glucose-regulated protein,” Mol. Cell. Biol., 8:4579, 1988.
Reuser, Kroos, Hermans, Bijvoet, Verbeet, Van Diggelen, Kleijer and Van der Ploeg, “Glycogenosis type II (acid maltase deficiency),” Muscle Nerve, 3:S61-69, 1995.
Ripe, Lorenzen, Brenner and Breindl, “Regulatory elements in the 5′ flanking region and the first intron contribute to transcriptional control of the mouse α-1-type collagen gene,” Mol. Cell. Biol., 9:2224, 1989.
Rippe et al., “DNA-mediated gene transfer into adult rat hepatocytes in primary culture,” Mol. Cell Biol., 10:689-695, 1990.
Rittling, Coutinho, Amarm and Kolbe, “AP-1/jun-binding sites mediate serum inducibility of the human vimentin promoter,” Nuc. Acids Res., 17:1619, 1989.
Rosen, Sodroski and Haseltine, “The location of cis-acting regulatory sequences in the human T-cell lymphotropic virus type III (HTLV-111/LAV) long terminal repeat,” Cell, 41:813, 1988.
Rucker, Fraites, Jr., Porvasnik, Lewis, Zolotukhin, Cloutier and Byrne, “Rescue of enzyme deficiency in embryonic diaphragm in a mouse model of metabolic myopathy: Pompe disease,” Development, 131:3007-19, 2004.
Rutledge, Halbert and Russell, “Infectious clones and vectors derived from adeno-associated virus (AAV) serotypes other than AAV type 2.” J. Virol., 72:309-19, 1998.
Sakai, Helms, Carlstedt-Duke, Gustafsson, Rottman and Yamamoto, “Hormone-mediated repression: A negative glucocorticoid-response element from the bovine prolactin gene,” Genes and Dev., 2:1144, 1988.
Sakamoto et al., “Micro-dystrophin cDNA ameliorates dystrophic phenotypes when introduced into mdx mice as a transgene,” Biochem. Biophys. Res. Commun., 293:1265-72, 2002.
Sambrook et al., “Molecular Cloning: A Laboratory Manual,” Cold Spring Harbor Laboratory, Cold Spring Harbor, N.Y., 1989.
Satake, Furukawa and Ito, “Biological activities of oligonucleotides spanning the F9 point mutation within the enhancer region of polyoma virus DNA,” J. Virology, 62:970, 1988.
Schaffner, Schirm, Muller-Baden, Wever and Schaffner, “Redundancy of information in enhancers as a principle of mammalian transcription control,” J. Mol. Biol., 201:81, 1988.
Sculier et al., “Pilot study of amphotericin B entrapped in sonicated liposomes in cancer patients with fungal infections,” J. Cancer Clin. Oncol., 24(3):527-538, 1988.
Searle, Stuart and Palmiter, “Building a metal-responsive promoter with synthetic regulatory elements,” Mol. Cell. Biol., 5:1480, 1985.
Segal, “Biochemical Calculations” 2nd Edition. John Wiley & Sons, New York, 1976.
Seiler et al., “Thixotropic solutions enhance viral-mediated gene transfer to airway epithelia,” Am. J. Respir. Cell Mol. Biol., 27:133-40, 2002.
Sharp and Marciniak, “HIV Tar: An RNA enhancer?” Cell, 59:229, 1989.
Shaul and Ben-Levy, “Multiple nuclear proteins in liver cells are bound to Hepatitis B virus enhancer element and its upstream sequences,” EMBO J., 6:1913, 1987.
Sherman, Basta, Moore, Brown and Ting, “Class II box consensus sequences in the HLA-DRα gene: Transcriptional function and interaction with nuclear proteins,” Mol. Cell. Biol., 9:50, 1989.
Shi and Bartlett, “RGD inclusion in VP3 provides adeno-associated virus type 2 (AAV2)-based vectors with a heparan sulfate-independent cell entry mechanism,” Mol. Ther., 7:515-25, 2003.
Shi, Arnold and Bartlett, “Insertional mutagenesis of the adeno-associated virus type 2 (AAV2) capsid gene and generation of AAV2 vectors targeted to alternative cell-surface receptors,” Hum. Gene Ther., 12:1697-711, 2001.
Sleigh and Lockett, “SV40 enhancer activation during retinoic-acid-induced differentiation of F9 embryonal carcinoma cells,” J. EMBO, 4:3831, 1985.
Spalholz, Yang and Howley, “Transactivation of a bovine papilloma virus transcriptional regulatory element by the E2 gene product,” Cell, 42:183, 1985.
Spandau and Lee, “Trans-activation of viral enhancers by the Hepatitis B virus X protein,” J. Virology, 62:427, 1988.
Spandidos and Wilkie, “Host-specificities of papilloma virus, Moloney murine sarcoma virus and simian virus 40 enhancer sequences,” EMBO J., 2:1193, 1983.
Stedman, Sweeney, Shrager, Maguire, Panettieri, Petrof, Narusawa, Leferovich, Sladky and Kelly, “The mdx mouse diaphragm reproduces the degenerative changes of Duchenne muscular dystrophy,” Nature, 352:536-39, 1991,
Stephens and Hentschel, “The bovine papilloma virus genome and its uses as a eukaryotic vector,“Biochem. J., 248:1, 1987.
Stuart, Searle and Palmiter, “Identification of multiple metal regulatory elements in mouse metallothionein-I promoter by assaying synthetic sequences,” Nature, 317:828, 1985.
Sullivan and Peterlin, “Transcriptional enhancers in the HLA-DQ subregion,” Mol. Cell. Biol., 7:3315, 1987.
Suzuki, Shin, Fjuikura, Matsuzaki, Takata, “Direct gene transfer into rat liver cells by in vivo electroporation,” FEBS Lett., 425(3):436-440, 1998.
Swartzendruber and Lehman, “Neoplastic differentiation: Interaction of simian virus 40 and polyoma virus with murine teratocarcinoma cells,” J. Cell. Physiology, 85:179, 1975.
Takakura, “Drug delivery systems in gene therapy,” Nippon Rinsho, 56(3):691-695, 1998.
Takebe, Seiki, Fujisawa, Hoy, Yokota, Arai, Yoshida and Arai, “SRα promoter: An efficient and versatile mammalian cDNA expression system composed of the simian virus 40 early promoter and the R-U5 segment of human T-cell leukemia virus Type 1 long terminal repeat,” Mol. Cell. Biol., 8:466, 1988.
Tavernier, Gheysen, Duerinck, Can der heyden and fiers, “Deletion mapping of the inducible promoter of human IFN-β gene,” Nature, 301:634, 1983.
Taylor and Kingston, “Ela trans-activation of human HSP70 gene promoter substitution mutants is independent of the composition of upstream and TATA elements,” Mol. Cell. Biol., 10:176, 1990b.
Taylor and Kingston, “Factor substitution in a human HSP70 gene promoter: TATA-dependent and TATA-independent interactions,” Mol. Cell. Biol., 10:165, 1990a.
Taylor, Solomon, Weiner, Paucha, Bradley and Kingston, “Stimulation of the human heat-shock protein 70 promoter in vitro by simian virus 40 large T antigen,” J. Biol. Chem., 264:15160, 1989.
Thiesen, Bosze, Henry and Chamay, “A DNA element responsible for the different tissue specificities of friend and Moloney retroviral enhancers,” J. Virology, 62:614, 1988.
Treisman, “identification of a protein-binding site that mediates transcriptional response to the c-fos gene to serum factors,” Cell, 46(4):567-574, 1986.
Tronche, Rollier, Bach, Weiss and Yaniv, “The rat albumin promoter: Cooperation with upstream elements is required when binding of APF/HNF 1 to the proximal element is partially impaired by mutation or bacterial methylation,” Mol. Cell. Biol., 9:4759, 1989.
Tronche, Rollier, Herbornel, Bach, Cereghini, Weiss and Yaniv, “Anatomy of the rat albumin promoter,” Mol. Biol. Med., 7:173, 1990.
Trudel and Constantini, “A 3′ enhancer contributes to the stage-specific expression of the human β-globin gene,” Genes and Dev., 6:954, 1987.
Tur-Kaspa et al., “Use of electroporation to introduce biologically active foreign genes into primary rat hepatocytes,” Mol. Cell Biol., 6:716-718, 1986.
Tyndall, La Mantia, Thacker, Favaloro and Kamen, “A region of the polyoma virus genome between the replication origin and late protein-coding sequences is required in cis for both early gene expression and viral DNA replication,” Nuc. Acids. Res., 9:6231, 1981.
Van Belle et al., “Effects of poloxamer 407 on transfection time and percutaneous adenovirus-mediated gene transfer in native and stented vessels,” Hum. Gene Ther., 9:1013-24, 1998.
Van Cott, Lubon, Russell, Butler, Gwazdauskas, Knight, Drohan, Velander, “Phenotypic and genotypic stability of multiple lines of transgenic pigs expressing recombinant human protein C,” Transgenic Res., 6(3):203-212, 1997.
Vanbever, Fouchard, Jadoul, De Morre, Preat, Marty, “In vivo noninvasive evaluation of hairless rat skin after high-voltage pulse exposure,” Skin Parmacol. Appl. Skin Physiol., 11(1):23-34, 1998.
Vannice and Levinson, “Properties of the human Hepatitis B virus enhancer: Position effects and cell-type nonspecificity,” J. Virology, 62:1305, 1988.
Vasseur, Kress, Montreau and Blangy, “Isolation and characterization of polyoma virus mutants able to develop in multipotential murine embryonal carcinoma cells,” Proc. Natl. Acad. Sci. U.S.A., 77:1068, 1980.
Wagner, Zatloukal, Cotten, Kirlappos, Mechtler, Curiel, Birnstiel, “Coupling of adenovirus to transferrin-polylysine/DNA complexes greatly enhances receptor-mediated gene delivery and expression of transfected genes,” Proc. Natl. Acad. Sci. USA, 89(13):6099-6103, 1992.
Wagner, Nepomuceno, Messner, Moran, Batson, Dimiceli, Brown, Desch, Norbash, Conrad, Guggino, Flotte, Wine, Carter, Reynolds, Moss and Gardner, “A phase II, double-blind, randomized, placebo-controlled clinical trial of tgAAVCF using maxillary sinus delivery in patients with cystic fibrosis with antrostomies,” Hum. Gene Ther., 13:1349-59, 2002.
Walker et al., “Strand displacement amplification—an isothermal, in vitro DNA amplification technique,” Nucleic Acids Res., 20(7): 1691-6, 1992.
Wang and Calame, “SV40 enhancer-binding factors are required at the establishment but not the maintenance step of enhancer-dependent transcriptional activation,” Cell, 47:241, 1986.
Wang, Louboutin, Nichols and Wilson, “Novel pseudotypes AAV vectors for hemophilia B gene therapy,” Mol. Ther., 9:S61-62, 2004.
Weber, De Villiers and Schaffner, “An SV40 ‘enhancer trap’ incorporates exogenous enhancers or generates enhancers from its own sequences,” Cell, 36:983, 1984.
Weinberger, Jat and Sharp, “Localization of a repressive sequence contributing to B-cell specificity in the immunoglobulin heavy-chain enhancer,” Mol. Cell. Biol., 8:988, 1984.
Winoto and Baltimore, “αβ-lineage-specific expression of the a T-cell receptor gene by nearby silencers,” Cell, 59:649, 1989.
Wong and Neumann, “Electric field mediated gene transfer,” Biochim. Biophys. Res. Commun., 107(2):584-587, 1982.
Wu and Dean, “Functional significance of loops in the receptor binding domain of Bacillus thuringiensis CryIIIA delta-endotoxin,” J. Mol. Biol., 255(4):628-640, 1996.
Wu and Wu, “Evidence for targeted gene delivery to HepG2 hepatoma cells in vitro,” Biochemistry, 27:887-892, 1988.
Wu and Wu, “Receptor-mediated in vitro gene transfections by a soluble DNA carrier system,” J. Biol. Chem., 262:4429-4432, 1987.
Wu et al., “Mutational analysis of the adeno-associated virus type 2 (AAV2) capsid gene and construction of AAV2 vectors with altered tropism,” J. Virol., 74:8635-47, 2000.
Xiao et al., “Gene therapy vectors based on adeno-associated virus type 1,” J. Virol., 73:3994-4003, 1999.
Yang, Lochmuller, Luo, Massie, Nalbantoglu, Karpati and Petrof, “Adenovirus-mediated dystrophin minigene transfer improves muscle strength in adult dystrophic (MDX) mice,” Gene Ther., 5:369-79, 1998.
Yang et al., “In vivo and in vitro gene transfer to mammalian somatic cells by particle bombardment,” Proc. Natl. Acad. Sci. USA, 87:9568-9572, 1990.
Yue, Li, Harper, Davisson, Chamberlain and Duan, “Microdystrophin gene therapy of cardiomyopathy restores dystrophin-glycoprotein complex and improves sarcolemma integrity in the mdx mouse heart,” Circulation, 108:1626-32, 2003.
Yutzey, Kline and Konieczny, “An internal regulatory element controls troponin I gene expression,” Mol. Cell. Biol., 9:1397, 1989.
Zabner, Seiler, Walters, Kotin, Fulgeras, Davidson and Chiorini, “Adeno-associated virus type 5 (AAV5) but not AAV2 binds to the apical surfaces of airway epithelia and facilitates gene transfer,” J. Virol., 74:3852-58, 2000.
Zambaux, Bonneaux, Gref, Maincent, Dellacherie, Alonso, Labrude, Vigneron, “Influence of experimental paparmeters on the characteristics of poly(lactic acid) nanoparticles prepared by a double emulsion method,” J. Controlled Release, 50(1-3):31-40, 1998.
Zolotukhin, Byrne, Mason, Zolotukhin, Potter, Chesnut, Summerford, Samulski and Muzyczka, “Recombinant adeno-associated virus purification using novel methods improves infectious titer and yield,” Gene Ther., 6:973-85, 1999.
Zolotukhin, Potter, Zolotukhin, Sakai, Loiler, Fraites, Jr., Chiodo, Phillipsberg, Muzyczka, Hauswirth, Flotte, Byrne and Snyder, “Production and purification of serotype 1, 2, and 5 recombinant adeno-associated viral vectors,” Methods, 28:158-67, 2002.
zur Muhlen, Schwarz, Mehnert, “Solid lipid nanoparticles (SLN) for controlled drug delivery—drug release and release mechanism,” Eur. J. Pharm. Biopharm., 45(2):149-155, 1998.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the, invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising:
(a) a recombinant adeno-associated viral vector that comprises a nucleic acid segment that encodes a mammalian therapeutic agent; and
(b) a water-soluble biocompatible gel.

2. The composition of claim 1, wherein said biocompatible gel comprises a sol, a matrix, a biogel, a hydrogel, a polymer, a polysaccharide, an oligosaccharide, or a viscous suspension.

3. The composition of claim 1, wherein said biocompatible gel comprises a polymer, a viscosity agent or a sucrose-based medium.

4. The composition of claim 1, wherein said biocompatible gel comprises iodixanol or sucrose acetate isobutyrate.

5. The composition of claim 1, wherein said biocompatible gel comprises glycerin, gelatin, or alginate.

6. The composition of claim 1, wherein said biocompatible gel comprises SAF-Gel, Duoderm Hydroactive Gel, Nu-Gel; Carrasyn (V) Acemannan Hydrogel, Elta Hydrogel or K-Y Sterile Gel.

7. The composition of claim 1, wherein said biocompatible gel comprises a cross-linked or a conjugated gel.

8. The composition of claim 1, wherein said recombinant adeno-associated viral vector is present in said composition at a concentration of at least 1×10¹² AAV particles per milliliter.

9. The composition of claim 1, wherein said biocompatible gel comprises at least about 85% by weight of said composition.

10. The composition of claim 9, wherein said biocompatible gel comprises at least about 95% by weight of said composition.

11. The composition of claim 1, wherein said mammalian therapeutic agent is a mammalian peptide, polypeptide, enzyme, protein, antisense, or ribozyme.

12. The composition of claim 1, wherein said mammalian therapeutic agent is a peptide, polypeptide, enzyme, protein, antisense, or ribozyme that can be expressed in human tissue.

13. The composition of claim 12, wherein said mammalian therapeutic agent is a peptide, polypeptide, enzyme, protein, antisense, or ribozyme that can be expressed in human cardiac or diaphragm muscle tissue.

14. The composition of claim 13, wherein said mammalian therapeutic agent is a biologically-active acid α-glucosidase (GAA), dystrophin, or α-1 antitrypsin polypeptide.

15. The composition of claim 1, further comprising a pharmaceutical excipient, buffer, carrier, or diluent.

16. A kit for diagnosing, preventing, treating or ameliorating the symptoms of a diseases or disorder in a mammal comprising: (i) the composition of claim 1; and (ii) instructions for using said kit.

17. A method of providing a biologically-effective amount of a therapeutic agent to a tissue site of a mammal in need thereof, said method comprising the step of providing to said mammal, the composition of claim 1, in an amount and for a time effective to provide said biologically-effective amount of said therapeutic agent to said tissue site of said mammal.

18. The method of claim 17, wherein said composition is provided to said mammal by infection, systemic administration, or by direct, indirect, or localized injection to a cell, tissue, or organ of said mammal.

19. The method of claim 17, wherein said mammal is human.

20. The method of claim 19, wherein said mammal is a human that has, is suspected of having, or at risk for developing a musculoskeletal disorder, a glycogen storage disease, or a congenital myopathy.

21. The method of claim 20, wherein said mammal is a human that has, is suspected of having, or at risk for developing muscular dystrophy, cardiac hypertrophy, or acid maltase deficiency (Pompe's Disease).

22. A method of treating or preventing a musculoskeletal disease or dysfunction, or a congenital myopathy in a mammal, said method comprising at least the step of providing to said mammal, the composition of claim 1, in an amount and for a time effective to treat or prevent said musculoskeletal disease or dysfunction, or said congenital myopathy in said mammal.

23. The method of claim 22, wherein said mammal is a human that has, is suspected of having, or at risk for developing muscular dystrophy.

24. A method of expressing in cells of a mammalian heart or diaphragm muscle, a nucleic acid segment that encodes an exogenously-provided mammalian therapeutic agent, said method comprising at least the step of directly injecting into said heart or said diaphragm muscle, the composition of claim 1, in an amount and for a time effective to express said exogenously-provided mammalian therapeutic agent.

25. A method for treating or ameliorating the symptoms of a congenital myopathy in a mammal, said method comprising administering to said mammal the composition of claim 1; in an amount and for a time sufficient to treat or ameliorate the symptoms of said congenital myopathy in said mammal.

26. The method of claim 25, wherein said congenital myopathy is muscular dystrophy.

27. A method for expressing a biologically-effective amount of an exogenously-supplied therapeutic polypeptide in a mammalian diaphragm, heart, or muscle cell, said method comprising: introducing into a population of said mammalian diaphragm, heart, or muscle cells, an amount of the composition of claim 1, for a time effective to express said biologically-effective amount of said exogenously-supplied therapeutic polypeptide in said mammalian diaphragm, heart or muscle cell.

28. The method of claim 27, wherein said therapeutic polypeptide is an enzyme, a kinase, a protease, a glucosidase, a glycosidase, a nuclease, a growth factor, a tissue factor, a myogenic factor, a neurotrophic factor, a neurotrophin, a dystrophin, an interleukin, or a cytokine.

29. The method of claim 28, wherein said therapeutic polypeptide is acid α-glucosidase (GAA).

30. The method of claim 27, wherein said composition is introduced into said population of said mammalian diaphragm, heart, or muscle cells by systemic, indirect, or localized infection, or by intramuscular, subcutaneous, intra-abdominal, transpleural, intracardiac, or transperitoneal injection.


SEQUENCE LISTING (12 sequences)

SEQ ID NO: 1 - DNA, 20 bp, Artificial
  Synthetic Oligonucleotide
agctggcgtaatagcgaaga

SEQ ID NO: 2 - DNA, 21 bp, Artificial
  Synthetic Oligonucleotide
cgcgtctctccaggtagcgaa

SEQ ID NO: 3 - DNA, 22 bp, Artificial
  Synthetic Oligonucleotide
cggtgatggtgctgcgttggag

SEQ ID NO: 4 - DNA, 20 bp, Artificial
  Synthetic Oligonucleotide
tcgacgttcagacgtagtgt

SEQ ID NO: 5 - DNA, 3846 bp, Homo sapiens
gcgcctgcgcgggaggccgcgtcacgtgacccaccgcggccccgccccgcgacgagctcccgccggtcacgtgacccgcctctgcgcgcccccgggcacgaccccggagtctccgcgggcggccagggcgcgcgtgcgcggaggtgagccgggccggggctgcggggcttccctgagcgcgggccgggtcggtggggcggtcggctgcccgcgccggcctctcagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgggcctgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggcctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgcttcccggctgcccagagggctggatgcctgccggtccccgagcaagcctgggaactcaggaaaattcacaggacttgggagattctaaatcttaagtgcaattattttaataaaaggggcatttggaatc

SEQ ID NO: 6 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 7 - DNA, 11066 bp, Homo sapiens
ttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagctgcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagagagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 8 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 9 - DNA, 22 bp, Artificial
  Synthetic Oligonucleotide
cggtgatggtgctgcgttggag

SEQ ID NO: 10 - DNA, 20 bp, Artificial
  Synthetic Oligonucleotide
tcgacgttcagacgtagtgt

SEQ ID NO: 11 - DNA, 19 bp, Artificial
  Synthetic Oligonucleotide
gctggtgaaaaggacctct

SEQ ID NO: 12 - DNA, 20 bp, Artificial
  Synthetic Oligonucleotide
cacaggactagaacacctgc